(19) United States
(12) Patent Application Publication
(10) Pub. No.: US 2004/0018497 A1
(43) Pub. Date: Jan. 29, 2004

(54) Human obesity LIPIN3 polynucleotide and polypeptide sequences and methods of use thereof

(76) Inventor: Craig H. Warden, Davis, CA (US)

Correspondence Address: Catherine M. Polizzi, Morrison & Foerster LLP, 755 Page Mill Road, Palo Alto, CA, 94304, US

(21) Appl. No.: 10/206,618

(22) Filed: Jul. 26, 2002

(51) Int. Cl. C12Q 1/68; C07H 21/04; C07K 14/775; C12P 21/02; C12N 5/06

(52) U.S. Cl. 435/6; 435/69.1; 435/320.1; 435/325; 530/359; 536/23.5

(57) ABSTRACT

The present invention relates to human LIPIN3 polynucleotides, which are involved in the control of mammalian body weight. The invention encompasses LIPIN3 polynucleotide sequences, including altered (mutant) LIPIN3 sequences, and fragments thereof, vectors and host cells comprising LIPIN3 polynucleotides, Lipin3 polypeptides, antibodies that specifically bind to Lipin3 polypeptide, arrays comprising LIPIN3 polynucleotides and polypeptides, and purified cells comprising an altered LIPIN3 polynucleotide. The invention also encompasses methods and compositions for the diagnosis and/or treatment of obesity and obesity related diseases, and for the identification of individuals susceptible to such diseases. The invention also encompasses methods of screening for compounds that alter LIPIN3 expression, preferably reducing LIPIN3 expression, and compounds that modulate the activity of the LIPIN3 gene product.

### STATEMENT OF RIGHTS TO INVENTIONS MADE UNDER FEDERALLY SPONSORED RESEARCH

Not applicable.

### TECHNICAL FIELD

This invention relates to the field of polynucleotides and polypeptides. More specifically, this invention relates to human LIPIN3 polynucleotides, Lipin3 polypeptides, and methods using these polynucleotides and polypeptides, especially for the detection, diagnosis, prevention and/or treatment of obesity and obesity related disorders, and methods of screening candidate anti-obesity drugs. This invention further relates to mutant LIPIN3 polynucleotides which are associated with obesity, and methods using these polynucleotides for the detection and diagnosis of obesity. This invention also relates to screening of the LIPIN3 gene for alterations, which are useful for identifying the predisposition to obesity and/or obesity-related diseases.

### BACKGROUND OF THE INVENTION

Obesity represents the most prevalent of body weight disorders, and it is the most important nutritional disorder in the western world, with estimates of its prevalence ranging from 30% to 50% within the middle-aged population. Other body weight disorders, such as anorexia nervosa and bulimia nervosa, which together affect approximately 0.2% of the female population of the western world, also pose serious health threats. Further, such disorders as anorexia and cachexia (wasting) are also prominent features of other diseases such as cancer, cystic fibrosis, and AIDS.

Obesity also contributes to other diseases. For example, this disorder is responsible for increased incidence of diseases such as coronary artery disease, hypertension, stroke, diabetes, hyperlipidemia, and some cancers. See, e.g., Nishina, P. M. et al., 1994, Metab. 43: 554-558; Grundy, S. M. & Barnett, J. P., 1990, Dis. Mon. 36: 641-731. Obesity is not merely a behavioral problem, i.e., the result of voluntary hyperphagia. Rather, the differential body composition observed between obese and normal subjects results from differences in both metabolism and neurologic/metabolic interactions. These differences seem to be, to some extent, due to differences in gene expression, and/or level of gene products or activity. Friedman, J. M. et al., 1991, Mammalian Gene 1: 130-144.

The epidemiology of obesity strongly shows that the disorder exhibits inherited characteristics. See Stunkard, 1990, N. Eng. J. Med. 322: 1438. Moll et al. have reported that, in many populations, obesity seems to be controlled by a few genetic loci. See Moll et al., 1991, Am. J. Hum. Gen. 49: 1243. In addition, human twin studies strongly suggest a substantial genetic basis in the control of body weight, with estimates of heritability of 80-90%. See Simopoulos, A. P. & Childs, B., eds., 1989, in “Genetic Variation and Nutrition in Obesity”, World Review of Nutrition and Diabetes 63, S. Karger, Basel, Switzerland; Borjeson, M., 1976, Acta. Paediatr. Scand. 65: 279-287. Studies of the genetics of human obesity, and of animal models of obesity demonstrate that obesity results from complex defective regulation of both food intake, food induced energy expenditure, and of the balance between lipid and lean body anabolism.

There are a number of genetic diseases in man and other species which feature obesity among their more prominent symptoms, along with, frequently, dysmorphic features and mental retardation. For example, Prader-Willi syndrome (hereinafter, “PWS”), reviewed in Knoll, J. H. et al., 1993, Am. J. Med. Genet. 46: 2-6, affects approximately 1 in 20,000 live births, and involves poor neonatal muscle tone, facial and genital deformities, and generally obesity.

In addition to PWS, many other pleiotropic syndromes have been characterized which include obesity as a symptom. These syndromes are genetically straightforward, and appear to involve autosomal recessive alleles. Such diseases include, among others, Ahlstroem, Carpenter, Bardet-Biedl, Cohen, and Morgagni-Stewart-Monel Syndromes.

A number of models exists for the study of obesity. See, e.g., Bray, G. A., 1992, Prog. Brain Res. 93: 333-341; and Bray, G. A., 1989, Amer. J. Clin. Nutr. 5: 891-902). For example, animals having mutations which lead to syndromes that include obesity symptoms have also been identified. Attempts have been made to utilize such animals as models for the study of obesity, and the best studied animal models to date for genetic obesity are mice. For reviews, see, e.g., Ravussin, E, and C. Bouchard, 2000, Eur. J. Pharmacol. 410:131-145; Friedman, J. M. et al., 1991, Mamm. Gen. 1: 130-144 and Friedman, J. M. and Liebel, R. L., 1992, Cell 69: 217-220.

Studies utilizing mice have confirmed that obesity is a very complex trait with a high degree of heritability. Mutations at a number of loci have been identified which lead to obese phenotypes. These include the autosomal recessive mutations obese (ob), diabetes (db), fat (fat), tubby (tub), agouti, leptin (db) and the mahogany gene. Polygenic obesity has been studied using quantitative trait loci (QTL) mapping and a number of QTLs linked to body weight or body fat in animals have been identified.

In summary, obesity, which poses a major, worldwide health problem, represents a complex, highly heritable trait. Given the severity, prevalence, and potential heterogeneity of such disorders, there exists a great need for the identification of those genes that participate in the control of body weight.

All publications cited herein are hereby incorporated by reference in their entirety.

### SUMMARY OF THE INVENTION

This invention provides human LIPIN3 gene polynucleotide sequences, Lipin3 polypeptides encoded by these sequences, antibodies that bind to these polypeptides, compositions comprising any of the above, as well as methods using the polynucleotides, polypeptides, and/or antibodies.

Accordingly, in one embodiment, the invention provides an isolated polynucleotide that comprises a polynucleotide having the sequence shown in Table 1, or its complement. The invention also provides an isolated polynucleotide that comprises a region of at least 10 contiguous polynucleotides shown in Table 1. An embodiment of the invention includes, for example, an isolated polynucleotide encoding a region of at least 15, at least 18, at least 20, at least 25, at least 30, at least 50, and at least 100 or more contiguous polynucleotides.

In other embodiments, the invention provides an isolated polynucleotide that comprises: (a) a polynucleotide comprising the sequence shown in Table 9, or its complement; or (b) a polynucleotide comprising amino acids 588-609 shown in Table 9, or its complement. The present invention also provides an isolated polynucleotide that comprises a polynucleotide encoding a region of the polypeptide shown in Table 9 or Table 7A, wherein said region is at least about 5 contiguous amino acids in length. For example, the invention includes an isolated polynucleotide encoding at least 10, at least 15, at least 20, at least 25, or more contiguous amino acids of a sequence depicted in Table 9 or Table 7A.

In another embodiment, the invention provides an isolated polynucleotide comprising (a) a polynucleotide encoding a region of at least 5 contiguous amino acids, said region comprising amino acids 438 and 439 shown in Table 9. In other embodiments, the invention provides an isolated polynucleotide comprising: (a) a polynucleotide encoding a region of at least about 5 contiguous amino acids, said region comprising amino acids 587 and 588 shown in Table 9. In further embodiments, the isolated polynucleotide encodes a region of at least 10, at least 15, or at least 20 or more contiguous amino acids.

In another embodiment, the invention encompasses LIPIN3 polynucleotides including full-length (unprocessed), processed, coding, non-coding or portions thereof.

The invention also includes polynucleotides that are able to hybridize to a sequence comprising a polynucleotide of the invention as discussed herein. In other embodiments, the invention provides an isolated polynucleotide comprising a sequence of at least 10 contiguous nucleotides (or more, such as 15, 18, 20, 25, 35, 40, 45, 50, 60, 75, or 100 contiguous nucleotides) that hybridizes with a polynucleotide (such as DNA or RNA) having the sequence depicted in Table 1 or Table 7B, or regions thereof as described above, under conditions where it does not hybridize with other polynucleotides from a mammalian cell, preferably a human cell, or under conditions in which hybridization to a polynucleotide having the sequence depicted in Table 1 or Table 7B is enriched relative to hybridization with other polynucleotides from a mammalian cell.

In other embodiment, the invention provides an isolated polynucleotide comprising the sequence shown in Table 8. The invention also provides an isolated polynucleotide comprising a region of at least about 10 contiguous nucleotides (or more, such as at least 15, 18, 20, 25, 35, 40, 45, 50, 60, 75 or 100 contiguous nucleotides), said region comprising nucleotide 1904 shown in Table 8.

Another embodiment of the invention provides an isolated polynucleotide having at least 85%, at least 88%, at least 90%, at least 92%, at least 94%, at least 96%, at least 98%, or at least 99% sequence identity with the polynucleotides of the invention as described herein. One embodiment provides an isolated polynucleotide having at least 85%, at least 88%, at least 90%, at least 92%, at least 94%, at least 96%, at least 98%, or at least 99% sequence identity with the sequence shown in Table 1. In other embodiments, isolated polynucleotides additionally have less than 85%, 83%, 80%, 75%, 70% sequence identity with the sequence of mouse LIPIN3 (available in GenBank Accession number NM022883).

In some embodiments, the polynucleotides of the invention further comprise a detectable label. In some embodiments, the polynucleotide of the invention is immobilized on a surface. In some embodiments of the invention, the polynucleotide of the invention is single stranded. In some embodiments of the invention, the polynucleotide of the invention is selected from the group consisting of DNA and RNA. In some embodiments of the invention, the polynucleotide of the invention is prepared in part by chemical synthesis.

It is understood that (unless otherwise specified or required), any embodiment of the invention described herein that is a polynucleotide encompasses both the double-stranded form and each of two complementary single-stranded forms known or predicted to make up the double stranded form.

It is further understood that the invention provides embodiments “having” or “consisting of” or “consisting essentially of” the polynucleotide, polypeptides, and/or antibodies described herein.

In another aspect, the invention provides vectors and expression vectors comprising any of the polynucleotides described herein.

In still other aspects, the invention provides a host cell comprising any of the polynucleotide or vectors described herein. In some embodiments, the present invention comprises substantially purified cells in which LIPIN3 expression is increased or decreased (or prevented or eliminated). As is understood in the art, a relative increase or decrease in LIPIN3 expression can be determined by comparison to suitable control cells. Another embodiment provides substantially purified cells in which LIPIN3 expression is inhibited or prevented. The cells are preferably of mammalian, primate, mouse, or human origin, but cells derived from other species may also be employed. The cells can comprise cells capable of stable growth through several passages in culture and/or cells grown for fewer or no passages in culture. The cells can be capable of sustained or indefinite growth in culture (for example, stable cell lines) or not capable of stable growth in culture (e.g., tissue explants).

The present invention also encompasses cells containing recombinant polynucleotides which comprises a LIPIN3 polynucleotide or a mutant LIPIN3 polynucleotide. In one embodiment, the invention provides a genetically engineered mammalian cell comprising a recombinantly modified LIPIN3 polynucleotide, such that the polynucleotide is overexpressed. In another embodiment, the invention provides cells comprising a mutant LIPIN3 polynucleotide. In another embodiment, a LIPIN3 polynucleotide is operatively linked to an inducible promoter. In still other embodiments, the genetically engineered cells possess a mutant LIPIN3 gene instead of a native LIPIN3 gene.

The invention also provides Lipin3 polypeptides. Therefore, the invention provides an isolated polypeptide comprising a polypeptide encoded by any of the polynucleotides of the invention, as described herein. In other embodiments, the present invention also provides an isolated polypeptide that comprises a region of the polypeptide shown in Table 9 or Table 7A, wherein said region is at least about 5 contiguous amino acids in length. For example, the invention includes at least 10, at least 15, at least 20, at least 25, or more contiguous amino acids of a sequence depicted in Table 9 or Table 7A. In some embodiments, the invention provides an isolated polypeptide comprising (a) a polypeptide having the sequence shown in Table 9; or (b) amino acids 588-609 shown in Table 9. In other embodiments, the invention provides an isolated polypeptide comprising a region of at least about 5 contiguous amino acids, said region comprising amino acids 438 and 439 shown in Table 9. In other embodiments, the invention provides an isolated polypeptide comprising a region of at least about 5 contiguous amino acids, said region comprising amino acids 587 and 588 shown in Table 9. In some embodiments, the region of the isolated polypeptide comprises at least about 10 contiguous amino acids, at least about 15 contiguous amino acids or at least about 20 contiguous amino acids.

In another embodiment, the invention provides an isolated polypeptide comprising a polypeptide having the sequence shown in Table 10. In another embodiment, the invention provides an isolated polypeptide comprising a region consisting at least about 5 contiguous amino acids, wherein said region includes amino acid 634 of Table 10.

In other embodiments, the invention provides any polypeptide described herein, wherein the polypeptide includes an epitope. In other embodiments, the invention provides any polypeptide described herein, wherein the polypeptide is immobilized on a solid support.

The invention also provides antibodies that bind Lipin3. Accordingly, the invention provides an antibody that binds to any of the Lipin3 polypeptides described herein. In some embodiments, the antibody binds to a peptide encoded by any of the polynucleotide described herein. In one embodiment, the invention provides an antibody capable of binding to a polypeptide of this invention. In another embodiment, the antibody is capable of specifically binding to a polypeptide comprising (a) a polypeptide comprising the sequence shown in Table 9; or (b) amino acids 588-609 shown in Table 9. The present invention also provides an antibody that is capable of binding to a region of the polypeptide shown in Table 9 or Table 7A, wherein said region is at least about 5 contiguous amino acids in length. For example, the invention includes an antibody that is capable of binding to at least 10, at least 15, at least 20, at least 25, or more contiguous amino acids of a sequence depicted in Table 9 or Table 7A.

In another embodiment, the invention provides an antibody that is capable of binding to a polypeptide comprising a region of at least 5 contiguous amino acids of the polypeptide shown in Table 9, said region comprising amino acids 438 and 439 shown in Table 9. In yet another embodiment, the invention provides an antibody that is capable of binding to a polypeptide comprising a region of at least 5 contiguous amino acids of the peptide shown in Table 9, said region comprising amino acids 587 and 588 shown in Table 9.

In another embodiment, the invention provides an antibody that is capable of binding to a polypeptide comprising a region of at least about 5 contiguous amino acids of the polypeptide sequence shown in Table 9, said region comprising amino acids 21 and 22 shown in Table 9. In another embodiment, the invention provides an antibody that is capable of binding to a polypeptide comprising a region of at least about 5 contiguous amino acids of the polypeptide sequence shown in Table 9, said region comprising amino acids 95 and 96 shown in Table 9. In another embodiment, the invention provides an antibody that is capable of binding to a polypeptide comprising a region of at least about 5 contiguous amino acids of the polypeptide sequence shown in Table 9, said region comprising amino acids 356 and 357 shown in Table 9. For example, the invention includes an antibody that is capable of binding to a polypeptide comprising a at least 10, at least 15, at least 20, at least 25, or more contiguous amino acids of a sequence depicted in Table 9 or Table 7A.

In one embodiment, the invention provides an antibody capable of binding to an altered (mutant) polypeptide of this invention. In one embodiment, the isolated polypeptide comprising a polypeptide having the amino acid sequence shown in Table 10, or a region consisting of at least about 5 contiguous amino acids (or more, e.g. about 10, about 15, 25, 50, 75, 100, 150, amino acids in length) of the amino acid sequence shown in Table 10, wherein said region includes amino acid 634.

In some embodiments, the antibody is a polyclonal antibody. In other embodiments, the antibody is a monoclonal antibody. In still other embodiments, the antibody is immobilized on a solid support. In still other embodiments, the antibody further comprises a detectable label.

In still other embodiments, the invention provides a recombinant cell comprising a recombinantly modified Lipin3 polynucleotide, such that said polynucleotide is overexpressed. In some embodiments, the invention provides a genetically engineered cell in which a native LIPIN3 polynucleotide sequence is disrupted so that expression of a native Lipin3 gene product is inhibited or prevented.

In other embodiments, the invention provides methods for detecting a Lipin3 polypeptide (i.e., a polypeptide of this invention) in a biological sample, in which the steps are: (a) contacting polypeptide from the sample with an anti-Lipin3 antibody described herein under conditions that permit the formation of a stable antigen-antibody complex and (b) detecting stable complexes formed, if any.

The invention also provides kits, arrays, and compositions comprising the polynucleotides, polypeptides and antibodies described herein. In one embodiment, the invention provides compositions for assessing LIPIN3 polynucleotide expression comprising any of the polynucleotides described herein. In another embodiment, the invention provides compositions for assessing LIPIN3 polynucleotide expression comprising a polynucleotide comprising a region of a polynucleotide having the sequence shown in Table 1 or Table 7B, wherein said region is at about 15 contiguous polynucleotides in length. In other embodiments, the invention provides compositions comprising any of the mutant LIPIN3 polynucleotides described herein.

In other embodiments, the invention provides compositions comprising any of the polypeptides described herein. In some embodiments, the invention provides compositions comprising a polypeptide comprising a region of a polypeptide having the sequence shown in Table 9 or Table 7A, wherein said region is at about 5 amino acids in length.

In another aspect, the invention provides an array comprising at least two LIPIN3 polynucleotides as described herein. In another embodiment, the invention provides an array comprising at least two polynucleotides each comprising a region of a polynucleotide having the sequence shown in Table 1, wherein said region is at least 10 contiguous nucleotides (or more, such as at least 15, 18, 20, 25, 35, 40, 45, 50, 60, 75 or 100 contiguous nucleotides). In another embodiment, the array further comprises at least one polynucleotide, or a region thereof, selected from the group consisting of a LIPIN1 polynucleotide, a LIPIN2 polynucleotide, a polynucleotide shown in Table 3, a polynucleotide shown in Table 4, and a polynucleotide shown in Table 5, a polynucleotide shown in Table 6, a polynucleotide shown in Table 7B.

In one embodiment of the present invention, an array comprises one or more isolated polynucleotides that specifically hybridize to the polynucleotide shown in Table 8, or to a region of at least about 10 contiguous nucleotides (or more, such as at least 15, 18, 20, 25, 35, 40, 45, 50, 60, 75 or 100 or more contiguous nucleotides) of the polynucleotide shown in Table 8, said region comprising nucleotide 1904 shown in Table 8. In another embodiment, the array further comprises at least one polynucleotide, or a region thereof, selected from the group consisting of a LIPIN1 polynucleotide, a LIPIN2 polynucleotide, a LIPIN3 polynucleotide as described herein, a polynucleotide shown in Table 3, a polynucleotide shown in Table 4, a polypeptide shown in Table 5, a polynucleotide shown in Table 6, and a polynucleotide shown in Table 7. In other embodiments, the array comprises one or more polynucleotide comprising a mutation described in Table 13 (and shown in FIG. 1).

In another embodiment, the invention provides arrays comprising at least two of any polypeptides described herein. In other embodiments, the invention provides arrays comprising at least two polypeptides each comprising a region of a polypeptide having the sequence shown in Table 9 or Table 7A, wherein said region is at about 5 amino acids in length. In other embodiments, the invention provides kits comprising: any polynucleotide described herein. In other embodiments, the invention further comprises instructions for using the polynucleotide(s) in any of the methods described herein. In other embodiments, the invention provides kits for detecting expression, including overexpression, of a LIPIN3 polynucleotide comprising: (a) any polynucleotide described herein; and (b) instructions for using the polynucleotide in any of the methods described herein. In other embodiments, the invention provides kits for detecting expression, including overexpression, of a LIPIN3 polynucleotide comprising: (a) any polynucleotide described herein; and (b) instructions for using the polynucleotide to assess LIPIN3 expression, including overexpression. In another embodiment, the invention provides kits for detecting overexpression of a LIPIN3 polynucleotide comprising: (a) a polynucleotide comprising a region of a polynucleotide having the sequence shown in Table 1 or Table 7B, wherein said region is at least about 15 polynucleotides in length; and (b) instructions for using the region to assess LIPIN3 expression, including overexpression. In still other embodiments, the invention provides kits for detecting a LIPIN3 polymorphisms associated with obesity comprising: a mutant polynucleotide as described herein.

In another aspect, the invention provides methods for detecting altered expression of a LIPIN3 polynucleotide in a test sample comprising detecting a level of expression of any of the LIPIN3 polynucleotide as described herein. In another embodiment, the invention provides methods for detecting altered expression of a LIPIN3 polynucleotide in a test sample comprising detecting a level of expression of any of the LIPIN3 polynucleotide as described herein; and determining whether the expression of the LIPIN3 polynucleotide is altered in the test sample compared to expression of a LIPIN3 polynucleotide in a normal sample. In other embodiments, the invention provides methods for detecting differential expression of a LIPIN3 polynucleotide in a test sample comprising: (a) detecting a level of expression of (i) a polynucleotide comprising the sequence shown in Table 1; (ii) a polynucleotide comprising the sequence shown in Table 7B; or (iii) a region of a polynucleotide comprising the sequence shown in Table 1 or Table 7B, wherein said region is at least about 10 nucleotides in length (or more, e.g., about 15, 18, 20, 25, 30, 35, 50, 100 or more nucleotides in length); (b) comparing the level of expression of the polynucleotide in the test sample with the level of expression of the polynucleotide in a control cell sample; and (c) determining the presence of differential expression of the polynucleotide in the test cell sample relative to the polynucleotide in the control cell sample, if any. In another embodiment, the invention provides methods for detecting differential expression of a LIPIN3 polynucleotide in a test sample comprising: (a) detecting a level of expression of at least one polynucleotide as described herein; (b) comparing the level of expression of the polynucleotide in the test sample with the level of expression of the polynucleotide in a control cell sample; and (c) determining the presence of differential expression of the polynucleotide in the test cell sample relative to the polynucleotide in the control cell sample, if any.

In another aspect, the invention provides methods for detecting (diagnosing, or aiding diagnosis of) obesity or obesity-related disorders associated with differential expression of a LIPIN3 polynucleotide comprising: (a) detecting a level of expression of any of the LIPIN3 polynucleotide as described herein. In some embodiments, the LIPIN3 polynucleotide comprises (i) a polynucleotide having the sequence shown in Table 1; (ii) a polynucleotide having the sequence shown in Table 7B; or (iii) a region of a polynucleotide having the sequence shown in Table 1 or Table 7B, wherein said region is at least about 10 nucleotides in length (or more, e.g., about 15, 18, 20, 25, 30, 35, 50, 100 or more nucleotides in length). In other embodiments, the invention provides methods for detecting (diagnosing, or aiding diagnosis of) obesity or obesity-related disorders associated with differential expression of a LIPIN3 polynucleotide comprising: (a) detecting a level of expression in a test sample of any of the LIPIN3 polynucleotide as described herein; and determining whether the expression of the LIPIN3 polynucleotide is altered in the test sample compared to expression of a LIPIN3 polynucleotide in a normal sample.

In other embodiments, the invention provides methods for detecting obesity or obesity-related disorders associated with differential expression of a LIPIN3 polynucleotide comprising: (a) detecting a level of expression of (i) a polynucleotide comprising the sequence shown in Table 1; (ii) a polynucleotide comprising the sequence shown in Table 7B; or (iii) a region of a polynucleotide comprising the sequence shown in Table 1 or Table 7B, wherein said region is at least about 10 nucleotides in length (or more, e.g., about 15, 18, 20, 25, 30, 35, 50, 100 or more nucleotides in length); (b) comparing the level of expression of the polynucleotide in the test sample with the level of expression of the polynucleotide in a control cell sample; and (c) determining the presence of differential expression of the polynucleotide in the test cell sample relative to the polynucleotide in the control cell sample, if any. In still another embodiment, the invention provides methods for detecting obesity or an obesity-related disorder associated with differential expression of a LIPIN3 polynucleotide in a test sample comprising: (a) detecting a level of expression of at least one polynucleotide as described herein; (b) comparing the level of expression of the polynucleotide in the test sample with the level of expression of the polynucleotide in a control cell sample; and (c) determining the presence of differential expression of the polynucleotide in the test cell sample relative to the polynucleotide in the control cell sample, if any.

In some embodiments of the methods described herein, the differential polynucleotide expression is elevated expression or decreased expression. In some embodiments of the methods described herein, the detecting is measuring the level of an RNA transcript. In some embodiments of the methods described herein, the detecting is by a method including PCR, TMA, bDNA, NAT, or Nasbau. In some embodiments of the methods described herein, the polynucleotide is immobilized on a surface.

In other embodiments, the invention provides methods of detecting a polynucleotide polymorphism (muttion) associated with obesity, comprising: detecting at least one mutant LIPIN3 polynucleotide as described herein. In other embodiments, the invention provides methods of diagnosing obesity or an obesity-related disorder, or aiding risk assessment of obesity or an obesity-related disorder (diesease) comprising: detecting at least one mutant LIPIN3 polynucleotide as described herein.

In some embodiments, the invention provides methods for detecting differential expression of a LIPIN3 polypeptide in a test sample comprising: (a) detecting a level of expression of (i) a polypeptide comprising the sequence shown in Table 9; (ii) a polypeptide comprising the sequence shown in Table 7A; or (iii) a polypeptide comprising a region of the polypeptide shown in Table 9 or Table 7A, wherein the region is at least 5 contiguous amino acids in length. In some embodiments, the invention provides methods for detecting differential expression of a LIPIN3 polypeptide in a test sample comprising: (a) detecting a level of expression of (i) a polypeptide comprising the sequence shown in Table 9; (ii) a polypeptide comprising the sequence shown in Table 7A; or (iii) a polypeptide comprising a region of the polypeptide shown in Table 9 or Table 7A, wherein the region is at least 5 contiguous amino acids in length; and determining whether the expression of the LIPIN3 polynucleotide is altered in the test sample compared to expression of a LIPIN3 polynucleotide in a normal sample. In some embodiments, the invention provides methods for detecting differential expression of a LIPIN3 polypeptide in a test sample comprising: (a) detecting a level of expression of (i) a polypeptide comprising the sequence shown in Table 9; (ii) a polypeptide comprising the sequence shown in Table 7A; or (iii) a polypeptide comprising a region of the polypeptide shown in Table 9 or Table 7A, wherein the region is at least 5 contiguous amino acids in length; (b) comparing the level of expression of the polypeptide in the test sample with the level of expression of the polypeptide in a control sample; and (c) determining the presence of differential expression of the polypeptide in the test cell sample relative to the polypeptide in the control sample, if any. In other embodiments, the invention provides methods for detecting differential expression of a LIPIN3 polypeptide in a test sample comprising: (a) detecting a level of expression of at least one polypeptide as described herein; (b) comparing the level of expression of the polypeptide in the test sample with the level of expression of the polypeptide in a control sample; and (c) determining the presence of differential expression of the polypeptide in the test cell sample relative to the polypeptide in the control sample, if any.

In some embodiments of the methods described herein, the differential expression is elevated expression or decreased expression. In some embodiments of the methods described herein, the detecting is by a method including western blotting, mass spectroscopy, ELISA, immunohistochemistry, and capillary electrophoresis.

In another embodiment, the invention provides methods of identifying an alteration in a LIPIN3 gene associated with obesity or an obesity related disorder comprising: comparing the sequence of the LIPIN3 gene or level of expression of the LIPIN3 gene in a tissue sample from a subject with the sequence of a wild-type LIPIN3 gene or level of expression of the wild-type LIPIN3 gene, wherein an alteration in the germline sequence or level of expression of the LIPIN3 gene of said subject is associated with obesity. In other embodiments, the invention further provides that the detection of an alteration comprises (a) screening for a specific alteration in the LIPIN3 gene in said sample; or (b) determining the level of expression of the LIPIN3 polynucleotide in said sample.

In other embodiments, the invention provides methods of screening for agents that reduce the expression of a LIPIN3 polynucleotide in a test cell sample comprising: contacting one or more cells possessing LIPIN3 expression (including overexpression) with an antiobesity drug candidate; measuring expression of any polynucleotide described herein; and determining whether LIPIN3 expression has decreased. In other embodiments, the invention provides that the amount of LIPIN3 polynucleotide is detected by detecting mRNA in said sample. In other embodiments, the level of LIPIN3 polynucleotide is measured by hybridizing said mRNA to a probe that specifically hybridizes to a LIPIN3 polynucleotide. In other embodiments, the hybridizing is according to a method selected from the group consisting of a Northern blot, a Southern blot, an array hybridization, an affinity chromatography, and an in situ hybridization. In still other embodiments, wherein the probe is a member of a plurality of probes that forms an array of probes. In still other embodiments, the level of LIPIN3 polynucleotide is measured using a nucleic acid amplification reaction.

In other embodiments, the invention provides methods of screening for agents that reduce the expression of a Lipin3 polypeptide in a test cell sample comprising: contacting one or more cells possessing LIPIN3 overexpression with an antiobesity drug candidate; measuring expression of any of the polypeptides described herein in the cell, and determining whether Lipin3 expression has decreased. In other embodiments, the amount of Lipin3 polypeptide is detected by detecting the level of a Lipin3 polypeptide. In still other embodiments, the detecting is via a method selected from the group consisting of capillary electrophoresis, a Western blot, mass spectroscopy, ELISA, immunochromatography, and immunohistochemistry.

The test cell can be cultured ex vivo, or can be an adipocyte. Further, the test agent can be administered to an animal comprising a cell containing the LIPIN3 nucleic acid or the lipin protein. The test agent can be selected from the group consisting of antibody; protein; nucleic acid; and small organic molecule.

### DESCRIPTION OF THE FIGURE

FIG. 1 (SEQ ID NO:1): shows genomic LIPIN3 polynucleotide sequences. The polynucleoide sequences are selected from the polynucleotide sequence disclosed in GenBank Accession No. NT 011382. Circled nucleotide indicate nucleotides at which a polymorphism was identified, as described in Example 5. Primer pairs and predicted intro-exon boundaries are marked on the sequence. Primer sequences are additionally shown in Table 12.

### MODES FOR CARRYING OUT THE INVENTION

We have discovered the human LIPIN3 coding region and have discovered that overexpression of mouse LIPIN3 is correlated with obesity in BSB mice, a model mouse having a spontaneous, multi-factorial obesity. This indicates that the discovery of agents that reduce LIPIN3 expression may have useful anti-obesity function. Further, we have identified human LIPIN3 mutant alleles that correlate with obesity.

Accordingly, the invention provides LIPIN3 polynucleotide sequences, including polynucleotides encoding the LIPIN3 gene product, Lipin3. These polynucleotide sequences are useful as probes, for example, for detecting the overexpression of LIPIN3 mRNA and for detection of mutant LIPIN3 gene sequences. They are also useful for producing Lipin3 polypeptide or regions thereof. The invention also provides Lipin3 polypeptides which are useful, inter alia, for making antibodies or for detection of altered expression of Lipin3 protein. Lipin3 polypeptides are also useful for the identification of Lipin3 binding proteins. Further, the invention provides antibodies raised against Lipin3 or regions of Lipin3. The invention also provides methods using the LIPIN3 polynucleotides of the invention, such as methods of detecting overexpression of LIPIN3 mRNA and methods of detecting LIPIN3 alleles associated with obesity. Other methods of the invention include screening methods for identifying agents that reduce expression of LIPIN3 mRNA in a cell. These and other embodiments will be described in more detail below.

Definitions

As used herein, “obesity” is a disease characterized by fat cell hypertrophy and hyperplasia. “Obesity” may be characterized by the presence of one or more obesity-related phenotypes, including, for example, increased body mass (as measured, for example, by body mass index, or “BMI”), altered anthropometry, basal metabolic rates, or total energy expenditure, chronic disruption of the energy balance, increased Fat Mass as determined, for example, by DEXA (Dexa Fat Mass percent), altered maximum oxygen use (VO2), high fat oxidation, high relative resting rate, glucose resistance, hyperlipidemia, insulin resistance, and hyperglycemia. See also, for example, Hopkinson et al. (1997) Am J Clin Nutr 65(2): 432-8 and Butte et al. (1999) Am J Clin Nutr 69(2): 299-307.

An “obesity-related disease” means a disease which is associated with, related to, and/or directly or indirectly caused by obesity. Obesity-related diseases include type 2 diabetes, hyperlipidemia, and hyperinsulinemia.

As used herein, “LIPIN3” or “LIPIN3 sequence” refers to the human LIPIN3 nucleic acid sequences (polynucleotide) described herein. A partial sequence of LIPIN3 coding region is shown in Table 1. Unless otherwise specified, the terms “LIPIN3” and “human LIPIN3” are interchangeable. When referring to LIPIN3 (i.e., the LIPIN3 sequence) from another organism, such as M. musculus, the reference to LIPIN3 will include reference to that organism (for example, “mouse LIPIN3”). As is understood in the art, the LIPIN3 gene includes, not only the coding sequences, but also 5′ and 3′ flanking sequences. A “fragment” or a “region” of LIPIN3 is a portion of the LIPIN3 sequence Preferably, a region of LIPIN3 comprises at least 10 contiguous nucleotides, more preferably at least 15, more preferably at least 18, more preferably at least 25, more preferably at least 30, more preferably at least 50, more preferably at least 100, more preferably at least 150, more preferably at least 200, more preferably at least 250, more preferably at least 300 contiguous nucleotides.

“Lipin3” refers to a protein (polypeptide) product encoded in the human LIPIN3 sequence. The sequence of Lipin3 is shown in Table 9. Unless otherwise specified, the terms “Lipin3” and “human Lipin3” are interchangeable. When referring to Lipin3 (i.e., the product of the LIPIN3 gene) in another organism (for example, Mus musculus) the reference to Lipin3 will refer to that organism (i.e., “mouse Lipin3”). A “region” of full-length Lipin3 is a portion of the LIPIN3 sequence. It is understood that Lipin3 may exist in more than one form, such as a single Lipin3 polypeptide, an assembly of at least one Lipin3 polypeptide, and/or within a complex (i.e., comprising multi-subunits) containing at least one Lipin3 polypeptide with at least one other polypeptide.

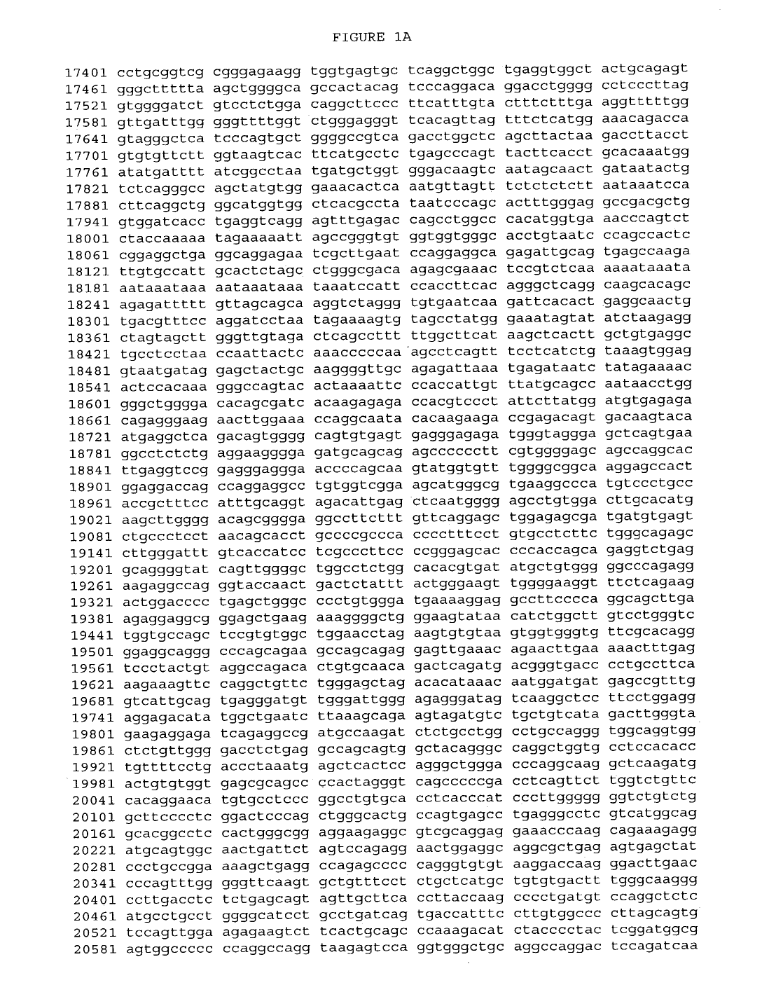

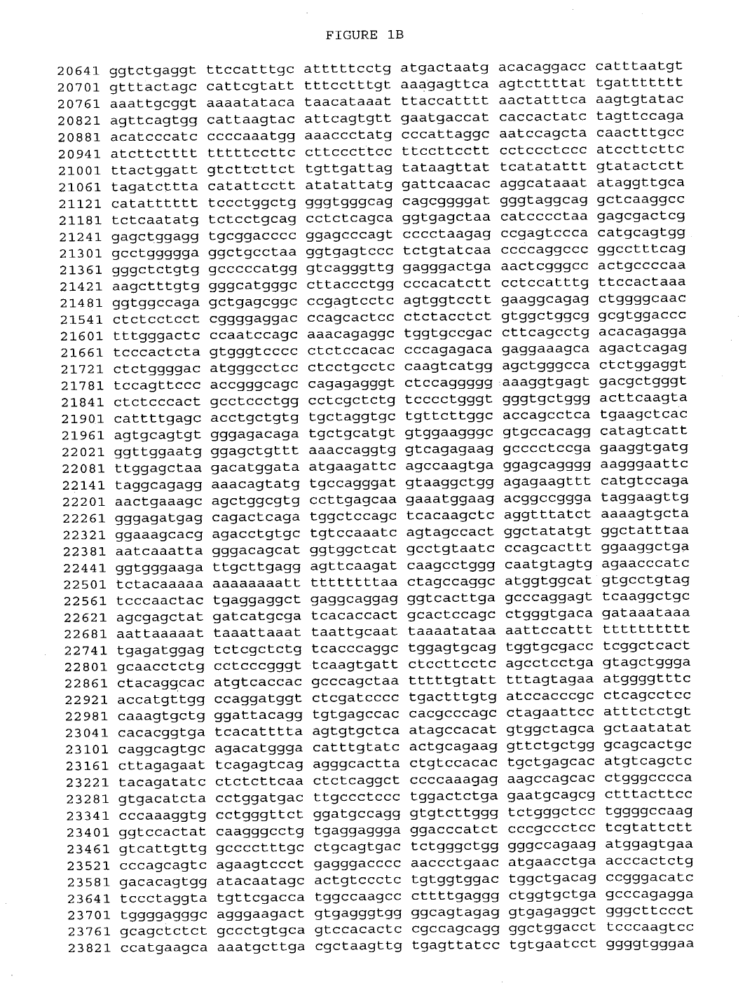

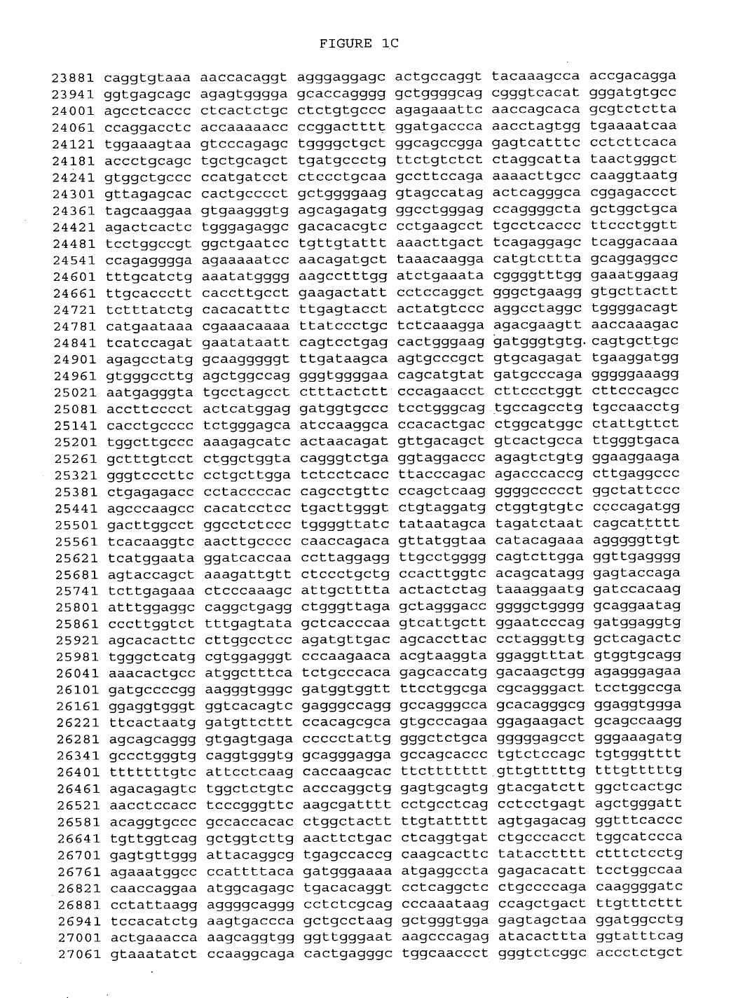

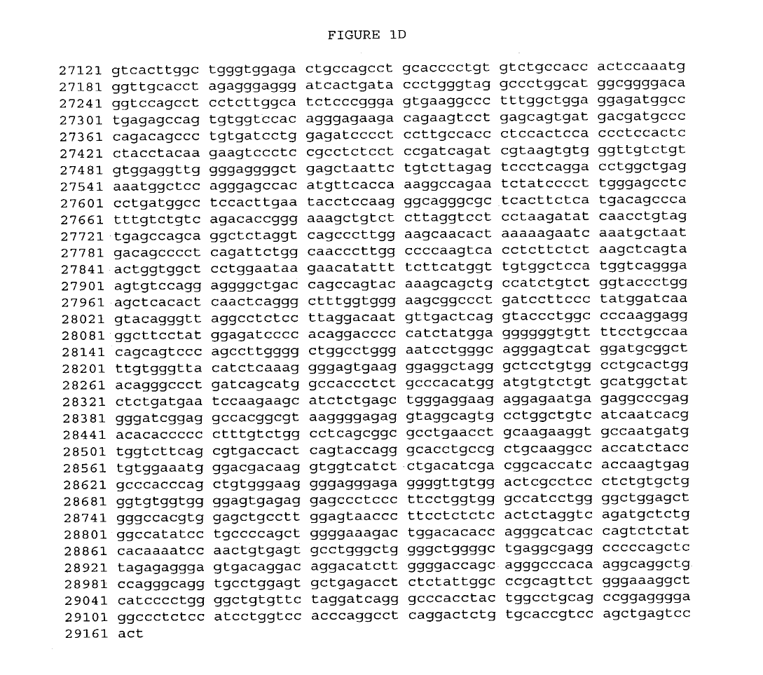

A “LIPIN3 polynucleotide” refers to any of the polynucleotide embodiments described herein and is based on the Lipin3 coding region (Table 1), but also includes 5′ and 3′ flanking sequences and non-coding sequences. A predicted organization of a partial LIPIN3 genomic region is depicted in FIG. 1. A “Lipin3 polypeptide” refers to a polypeptide product encoded by or within LIPIN3; thus, a “Lipin3 polypeptide” refers to any of the polypeptide embodiments described herein, including full-length Lipin3.

As used herein, a “polynucleotide” is a polymeric form of nucleotides of any length, which contain deoxyribonucleotides, ribonucleotides, and/or their analogs. The terms “polynucleotide” and “nucleotide” as used herein are used interchangeably. Polynucleotides may have any three-dimensional structure, and may perform any function, known or unknown. The term “polynucleotide” includes double-, single-stranded, and triple-helical molecules. Unless otherwise specified or required, any embodiment of the invention described herein that is a polynucleotide encompasses both the double-stranded form and each of two complementary single-stranded forms known or predicted to make up the double stranded form. Not all linkages in a polynucleotide need be identical.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. The use of uracil as a substitute for thymine in a deoxyribonucleic acid is also considered an analogous form of pyrimidine.

If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications included in this definition are, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ploy-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s).

Further, any of the hydroxyl groups ordinarily present in the sugars may be replaced by phosphonate groups, phosphate groups, protected by standard protecting groups, or activated to prepare additional linkages to additional nucleotides, or may be conjugated to solid supports. The 5′ and 3′ terminal OH groups can be phosphorylated or substituted with amines or organic capping group moieties of from 1 to 20 carbon atoms. Other hydroxyls may also be derivatized to standard protecting groups.

Polynucleotides can also contain analogous forms of ribose or deoxyribose sugars that are generally known in the art, including, but not limited to, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside.

Although conventional sugars and bases will be used in applying the method of the invention, substitution of analogous forms of sugars, purines and pyrimidines can be advantageous in designing a final product, as can alternative backbone structures like a polyamide backbone.

A polynucleotide or polynucleotide region has a certain percentage (for example, 80%, 85%, 90%, or 95%) of “sequence identity” to another sequence means that, when aligned, that percentage of bases are the same in comparing the two sequences. This alignment and the percent homology or sequence identity can be determined using software programs known in the art, for example those described in Current Protocols in Molecule Biology (F. M. Ausubel et al., eds., 1987) Supplement 30, section 7.718, Table 4.7.1. Percent identity can be determined electronically, e.g., by using the MegAlign.TM. program (DNASTAR, Inc., Madison Wis.). The MegAlign.TM. program can create alignments between two or more sequences according to different methods, e.g., the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol. 183:626-645.)

A nucleotide is said to “encode” a polypeptide if, in its native state or when manipulated by methods well known to those skilled in the art, it can be transcribed and/or translated to produce the polypeptide or a fragment thereof. For purposes of this invention, and to avoid cumbersome referrals to complementary strands, the anti-sense (or complementary) strand of such a polynucleotide is also said to encode the sequence; that is, a polynucleotide sequence that “encodes” a polypeptide includes both the conventional coding strand and the complementary sequence (or strand).

A “primer” is a short polynucleotide, generally with a free 3′-OH group, that binds to a target potentially present in a sample of interest by hybridizing with the target, and thereafter promoting polymerization of a polynucleotide complementary to the target.

A “probe” when used in the context of polynucleotide manipulation refers to a polynucleotide which is provided as a reagent to detect a target potentially present in a sample of interest by hybridizing with the target. A probe may comprise a label or a means by which a label can be attached, either before or subsequent to the hybridization reaction. Suitable labels include, but are not limited to radioisotopes, fluorochromes, chemiluminescent compounds, dyes, and enzymes.

A “mutant” LIPIN3 polynucleotide or polypeptide is a polynucleotide or polypeptide sequence that comprises one or more deletions, addition, transversion, or alteration in nucleic acid or amino acid sequence. As described further herein, a mutant LIPIN3 or Lipin3 sequence may result in a truncated or altered LIPIN3 polynucleotide or polypeptide, increased or decreased expression of a LIPIN3 polynucleotide or polypeptide, or any combination thereof. The mutation may be in coding, non-coding, 5′ or 3′ flanking, genomic or coding nucleotides.

“Transformation” or “transfection” refers to the insertion of an exogenous polynucleotide into a host cell, irrespective of the method used for the insertion, for example, lipofection, transduction, infection or electroporation. The exogenous polynucleotide may be maintained as a non-integrated vector, for example, a plasmid, or alternatively, may be integrated into the host cell genome.

The terms “polypeptide”, “oligopeptide”, “peptide” and “protein” are used interchangeably herein to refer to polymers of amino acids of any length. The polymer may be linear or branched, it may comprise modified amino acids, it may be interrupted by non-amino acids, and it may be assembled into a complex of more than one polypeptide chain. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

A polypeptide “fragment” (also called a “region”) of Lipin3 (or a “Lipin3 fragment” or “Lipin3 region”) is a polypeptide comprising an amino acid sequence of Lipin3 that has at least 5 contiguous amino acids of a sequence of Lipin3, more preferably at least 10 contiguous amino acids, more preferably at least about 15 contiguous amino acids, even more preferably at least about 25 contiguous amino acids, even more preferably at least about 30 contiguous amino acids, even more preferably at least about 40 contiguous amino acids. For purposes of this invention, it is understood that a Lipin3 fragment does not have the same amino acid sequence as M. musculus Lipin3.

A “fusion polypeptide” is a polypeptide comprising regions in a different position than occurs in nature. The regions may normally exist in separate proteins and are brought together in the fusion polypeptide, or they may normally exist in the same protein but are placed in a new arrangement in the fusion polypeptide.

A “functionally preserved” variant of a LIPIN3 polynucleotide or Lipin3 polypeptide is a LIPIN3 or Lipin3 sequence which retains at least one aspect of LIPIN3 function. Functionally preserved variants can be due to differences in linear sequence, arising from, for example, single base mutation(s), addition(s), deletion(s), and/or modification(s) of the bases. The difference can also arise from changes in the sugar(s) and/or linkage(s) between the bases. Regarding polypeptides, functionally preserved variants may arise, for example, by conservative and/or non-conservative amino acid substitutions, amino acid analogs, and deletions. The function that is preserved depends upon the relevant function being considered. For example, if a LIPIN3 polynucleotide is considered for a probe, then the ability of a variant polynucleotide sequence to hybridize to the target is the relevant function. If a polynucleotide is considered for its ability to encode a Lipin3 polypeptide (or fragment thereof), then the ability of a variant sequence to encode the same polypeptide is the relevant function. If a Lipin3 polypeptide is considered for its ability to bind to a particular entity (such as an antibody or another protein), then the ability of a variant sequence to encode a polypeptide with equivalent binding characteristics that is relevant. A Lipin3 polypeptide may be considered for its biological activity of the encoded gene product (e.g., a biological activity ascribed to a gene product corresponded to the LIPIN3 polynucleotides as a result of the assignment of the gene product to a protein family(ies) and/or identification of a functional domain present in the gene product).

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions.

“Expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

A “host cell” includes an individual cell or cell culture which can be or has been a recipient for vector(s) or for incorporation of nucleic acid molecules and/or proteins. Host cells include progeny of a single host cell, and the progeny may not necessarily be completely identical (in morphology or in genomic of total DNA complement) to the original parent cell due to natural, accidental, or deliberate mutation. A host cell includes cells transfected in vivo with a polynucleotide(s) of this invention.

As used herein, “expression” includes transcription and/or translation.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

“Heterologous” means derived from (i.e., obtained from) a genotypically distinct entity from the rest of the entity to which it is being compared. For example, a polynucleotide may be placed by genetic engineering techniques into a plasmid or vector derived from a different source, thus becoming a heterologous polynucleotide. A promoter which is linked to a coding sequence with which it is not naturally linked is a heterologous promoter.

An “isolated” or “purified” polynucleotide, polypeptide, antibody or cell is one that is substantially free of the materials with which it is associated in nature. By substantially free is meant at least 50%, preferably at least 70%, more preferably at least 80%, and even more preferably at least 90% free of the materials with which it is associated in nature. As used herein, an “isolated” polynucleotide or polypeptide also refers to recombinant polynucleotides or polypeptides, which, by virtue of origin or manipulation: (1) are not associated with all or a portion of a polynucleotide or polypeptide with which it is associated in nature, (2) are linked to a polynucleotide or polypeptide other than that to which it is linked in nature, or (3) does not occur in nature, or (4) in the case of polypeptides arise from expression of recombinant polynucleotides.

A “reagent” polynucleotide, polypeptide, or antibody, is a substance provided for a reaction, the substance having some known and desirable parameters for the reaction. A reaction mixture may also contain a “target”, such as a polynucleotide, antibody, polypeptide, or assembly of polypeptides that the reagent is capable of reacting with. For example, in some types of diagnostic tests, the presence and/or amount of the target in a sample is determined by adding a reagent, allowing the reagent and target to react, and measuring the amount of reaction product (if any). In the context of clinical management, a “target” may also be a cell, collection of cells, tissue, or organ that is the object of an administered substance, such as a pharmaceutical compound.

A “stable duplex” of polynucleotides, or a “stable complex” formed between any two or more components in a biochemical reaction, refers to a duplex or complex that is sufficiently long-lasting to persist between formation of the duplex or complex and subsequent detection, including any optional washing steps or other manipulation that may take place in the interim.

As used herein, the term “agent” means a biological or chemical compound such as a simple or complex organic or inorganic molecule, a peptide, a protein, oligonucleotide, polynucleotide, carbohydrate, or lipoprotein. A vast array of compounds can be synthesized, for example oligomers, such as oligopeptides and oligonucleotides, and synthetic organic compounds based on various core structures, and these are also included in the term “agent”. In addition, various natural sources can provide compounds for screening, such as plant or animal extracts, and the like. Compounds can be tested singly or in combination with one another.

A gene or polynucleotide is “differentially expressed” in a test sample when the polynucleotide is detected at a higher or lower level compared with a control sample of the same type. Typically, a differentially expressed polynucleotide includes polynucleotides that are expressed such that, for example, mRNA is found at levels at least about 25%, at least about 50% to 75%, at least about 90%, at least about 2-fold, at least about 4-fold, at least about 5-fold, and at least about 10-fold or more, higher (e.g. overexpressed) or lower (e.g., underexpressed). The comparison can be made between two tissue, for example, if one is using in situ hybridization or another assay method that allows some degree of discrimination among cell types in the tissue. The comparison may also be made between cells removed from their tissue source.

An “individual” is a vertebrate, preferably a mammal, more preferably a human. Mammals include, but are not limited to, farm animals, sport animals, primates, rodents, and pets.

An “antibody” is an immunoglobulin molecule-capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, humanized antibodies, chimeric antibodies, and any other modified configuration of the immunoglobulin molecule that comprises an antigen recognition site of the required specificity.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen binding site derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions.

As used herein, methods for “aiding diagnosis” means that these methods assist in making a clinical determination regarding the classification, or nature, of the obesity, and may or may not be conclusive with respect to the definitive diagnosis. Accordingly, a method of aiding diagnosis of obesity, or an obesity-related disease, can comprise the step of detecting the level of LIPIN3 expression in a biological sample from the individual. A method of aiding diagnosis of obesity, or an obesity-related disease, can also comprise the step of detecting altered levels of a LIPIN3 polynucleotide and/or polypeptide in a biological sample from the individual and/or detecting increased levels of a LIPIN3 polynucleotide and/or polypeptide in a biological sample from the individual.

An individual “at risk” may or may not have detectable disease, and may or may not have displayed detectable disease prior to the treatment methods described therein. “At risk” denotes that an individual who is determined to be more likely to develop a symptom based on conventional risk assessment methods or has one or more risk factors that correlate with development of obesity or an obesity-related disease. An individual having one or more of these risk factors has a higher probability of developing obesity or an obesity-related disease than an individual without these risk factors. Examples (i.e., categories) of risk groups are well known in the art and discussed herein.

“Development” or “progression” of obesity herein means initial manifestations and/or ensuing progression of the disorder. Development of obesity can be detectable and assessed using standard clinical techniques, such as measurement of increased body mass (as measured, for example, by body mass index, or “BMI”), altered anthropometry, basal metabolic rates, or total energy expenditure, chronic disruption of the energy balance, increased Fat Mass as determined, for example, by DEXA (Dexa Fat Mass percent), altered maximum oxygen use (VO2), high fat oxidation, high relative resting rate, glucose resistance, hyperlipidemia, insulin resistance, and hyperglycemia. See also, for example, Hopkinson et al. (1997) Am J Clin Nutr 65(2): 432-8 and Butte et al. (1999) Am J Clin Nutr 69(2): 299-307. However, development also refers to disease progression that may be undetectable. For purposes of this invention, development or progression refers to the biological course of the disease state. “Development” includes occurrence, recurrence, and onset. As used herein “onset” or “occurrence” of obesity includes initial onset and/or recurrence.

As used herein, “delaying development” of obesity, or an obesity-related disease, means to defer, hinder, slow, retard, stabilize, and/or postpone development of the disease. This delay can be of varying lengths of time, depending on the history of the disorder and/or the medical profile of the individual being treated. As is evident to one skilled in the art, a sufficient or significant delay can, in effect, encompass prevention, in that the individual does not develop detectable disease. A method that “delays” development of disease is a method that reduces the extent of the disease in a given time frame, when compared to not using the method. Such comparisons are typically based on clinical studies, using a statistically significant number of subjects, although this knowledge can be based upon anecdotal evidence. “Delaying development” can mean that the extent and/or undesirable clinical manifestations are lessened and/or time course of the progression is slowed or lengthened, as compared to not administering the agent. Thus the term also includes, but is not limited to, alleviation of symptoms, diminishment of extent of disease, stabilized (i.e., not worsening) state of disease, delay or slowing of disease progression, and remission (whether partial or total) whether detectable or undetectable.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Wei & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); “Current Protocols in Immunology” (J. E. Coligan et al., eds., 1991).

Polynucleotides of the Invention

The present invention provides LIPIN3 polynucleotides, including LIPIN3 polynucleotides encoding human Lipin3 (i.e., a Lipin3 polypeptide), polynucleotides from the flanking region(s) or intron regions of LIPIN3, altered LIPIN3 polynucleotides (mutant alleles), vectors containing these polynucleotides, host cells containing these polynucleotides, arrays comprising these polynucleotides, and compositions comprising these polynucleotides. These polynucleotides are isolated and/or produced by chemical and/or recombinant methods, or a combination of these methods. Unless specifically stated otherwise, the term “polynucleotides” shall include all embodiments of the polynucleotides of this invention. It is understood that (unless otherwise specified or required), any embodiment of the invention described herein that is a polynucleotide encompasses both the double-stranded form and each of two complementary single-stranded forms known or predicted to make up the double stranded form.

The identification of a partial LIPIN3 polynucleotide coding sequence is described in Example 1 and shown in Table 1.

TABLE 1
Partial LIPIN3 polynucleotide coding sequence.

(SEQ ID NO:2)

| | |
|---|---|
| atgaactacg tggggcagct ggcggagacg gtgtttggga cggtgaagga gctgtaccgg | 60 |
| ggcctgaacc cagccacact gagcggcggc attgacgtgc tggtggtgaa gcaggtggac | 120 |
| ggctcgttcc ggtgctcacc cttccacgtg cgttttggca agctgggcgt cctgcggtcg | 180 |
| tagacattga gctcaatggg gagcctgtgg acttgcacat gaagcttggg gacagcgggg | 240 |
| aggccttctt tgttcaggag ctggagagcg acctctcagc aggtgagcta acatccccta | 300 |
| agagcgactc ggagctggag gtgcggaccc cggagcccag tcccctaaga gccgagtccc | 360 |
| acatgcagtg ggcctggggg aggctgccta actccccaaa gagaagccag cacctgggcc | 420 |
| ccagtgacat ctacctggat gacttgccct ccctggactc tgagaatgca gcgctttact | 480 |
| tccactctgg gctgggggcc agaagatgga gtgaacccag cagtcagaag tccctgaggg | 540 |
| accccaaccc tgaacatgaa cctgaaccca ctctggacac agtggataca atagcactgt | 600 |
| ccctctgtgg tggactggct gacagccggg acatcgtgcc cagagaaatt caaccagcac | 660 |
| agcgtctctt accaggacct caccaaaaac cccggacttt tggatgaccc aaacctagtg | 720 |
| gtgaaaatca acattataac tgggctgtgg ctgcccccat gatcctctcc ctgcaagcct | 780 |
| tccagaaaaa cttgcccaag gtaatggcac catggacaag ctggagaggg agaagatgcc | 840 |
| ccggaagggt gggcgatggt ggttttcctg gcgacgcagg gacttcctgg ccgagagaag | 900 |
| acagaagtcc tgagcagtga tgacgatgcc ccagacagcc ctgtgatcct ggagatcccc | 960 |
| tccttgccac cctccactcc accctccact cctacctaca agaagtccct ccgcctctcc | 1020 |
| tccgatcaga tcggcgcctg aacctgcaag aaggtgccaa tgatgtggtc ttcagcgtga | 1080 |
| ccactcagta ccagggcacc tgccgctgca aggccaccat ctacctgtgg aaatgggacg | 1140 |
| acaaggtggt catctctgac atcgacggca ccatctcact ctaggtcaga tgctctgggc | 1200 |
| catatcctgc cccagctggg gaaagactgg acacaccagg gcatcaccag tctctatcac | 1260 |
| aaaatccaac tgtgagt 1277 | |

The identification of a partial LIPIN3 genomic region is described in Example 1 and shown in FIG. 1.

The polynucleotides may comprise any novel region (i.e., not disclosed in the public domain as of the filing date of the original application of this series) of the sequence shown in Table 1 and FIG. 1. The polynucleotides of this invention, including fragments of polynucleotides of this invention, are useful as probes, primers, in expression systems, and in screening systems, especially screening systems for agents that modulate LIPIN3 expression, and preferably reduced LIPIN3 expression. Especially useful applications of the polynucleotides will be discussed below.

As noted above, the polynucleotides of the invention include polynucleotides encoding the Lipin3 polypeptide sequence described herein. Identification of Lipin3 polypeptide sequence is described in Example 1, and shown in Table 2. Lipin3 polypeptides are further described infra.

TABLE 2
Lipin3 amino acid sequence (609 amino acids).

| | |
|---|---|
| Met Asn Tyr Val Gly Gln Leu Ala Glu Thr Val Phe Gly Thr Val Lys | (SEQ ID NO:3) |
| 1 5 10 15 | |
| Glu Leu Tyr Arg Gly Leu Asn Pro Ala Thr Leu Ser Gly Gly Ile Asp | |
| 20 25 30 | |
| Val Leu Val Val Lys Gln Val Asp Gly Ser Phe Arg Cys Ser Pro Phe | |
| 35 40 45 | |
| His Val Arg Phe Gly Lys Leu Gly Val Leu Arg Ser Arg Gln Lys Val | |
| 50 55 60 | |
| Asp Ile Glu Leu Asn Gly Glu Pro Val Asp Leu His Met Lys Leu Gly | |
| 65 70 75 80 | |
| Asp Ser Gly Glu Ala Phe Phe Val Gln Glu Leu Gln Ser Asp Asp Leu | |
| 85 90 95 | |
| Ser Ala Gly Glu Leu Thr Ser Pro Lys Ser Asp Ser Glu Leu Glu Val | |
| 100 105 110 | |
| Arg Thr Pro Glu Pro Ser Pro Leu Arg Ala Glu Ser His Met Gln Trp | |
| 115 120 125 | |
| Ala Trp Gly Arg Leu Pro Lys Gly Ser Pro Lys Arg Ser Gln His Leu | |
| 130 135 140 | |
| Gly Pro Ser Asp Ile Tyr Leu Asp Asp Leu Pro Ser Leu Asp Ser Glu | |
| 145 150 155 | |
| Asn Ala Ala Leu Tyr Phe Pro Gln Ser Asp Ser Gly Leu Gly Ala Arg | |
| 165 170 175 | |
| Arg Trp Ser Glu Pro Ser Ser Gln Lys Ser Leu Arg Asp Pro Asn Pro | |
| 180 185 190 | |
| Glu His Glu Pro Glu Pro Thr Leu Asp Thr Val Asp Thr Ile Ala Leu | |
| 195 200 205 | |
| Ser Leu Cys Gly Gly Leu Ala Asp Ser Arg Asp Ile Ser Leu Glu Lys | |
| 210 215 220 | |
| Phe Asn Gln His Ser Val Ser Tyr Gln Asp Leu Thr Lys Asn Pro Gly | |
| 225 230 235 240 | |
| Leu Leu Asp Asp Pro Asn Leu Val Val Lys Ile Asn Gly Lys His Tyr | |
| 245 250 255 | |
| Asn Trp Ala Val Ala Ala Pro Met Ile Leu Ser Leu Gln Ala Phe Gln | |
| 260 265 270 | |
| Lys Asn Leu Pro Lys Val Met Val Arg Glu His His Gly Gln Ala Gly | |
| 275 280 285 | |
| Glu Gly Glu Asp Ala Pro Glu Gly Trp Ala Met Val Val Phe Leu Ala | |
| 290 295 300 | |
| Thr Gln Gly Leu Pro Gly Arg Gly Gly Glu Lys Thr Glu Val Leu Ser | |
| 305 310 315 320 | |
| Ser Asp Asp Asp Ala Pro Asp Ser Pro Val Ile Leu Glu Ile Pro Ser | |
| 325 330 335 | |
| Leu Pro Pro Ser Thr Pro Pro Ser Thr Pro Thr Tyr Lys Lys Ser Leu | |
| 340 345 350 | |
| Arg Leu Ser Ser Asp Gln Ile Arg Arg Leu Asn Leu Gln Glu Gly Ala | |
| 355 360 365 | |
| Asn Asp Val Val Phe Ser Val Thr Thr Gln Tyr Gln Gly Thr Cys Arg | |
| 370 375 380 | |
| Cys Lys Ala Thr Ile Tyr Leu Trp Lys Trp Asp Asp Lys Val Val Ile | |
| 385 390 395 400 | |
| Ser Asp Ile Asp Gly Thr Ile Thr Lys Ser Asp Ala Leu Gly His Ile | |
| 405 410 415 | |
| Leu Pro Gln Leu Gly Lys Asp Trp Thr His Gln Gly Ile Thr Ser Leu | |
| 420 425 430 | |
| Tyr His Lys Ile Gln Leu Asn Gly Tyr Lys Phe Leu Tyr Cys Ser Ala | |
| 435 440 445 | |
| Arg Ala Ile Gly Met Ala Asp Leu Thr Lys Gly Tyr Leu Gln Trp Val | |
| 450 455 460 | |
| Ser Glu Gly Gly Cys Ser Leu Pro Lys Gly Pro Ile Leu Leu Ser Pro | |
| 465 470 475 480 | |
| Ser Ser Leu Phe Ser Ala Leu His Arg Glu Val Ile Glu Lys Lys Pro | |
| 485 490 495 | |
| Glu Val Phe Lys Val Ala Cys Leu Ser Asp Ile Gln Gln Leu Phe Leu | |
| 500 505 510 | |
| Pro His Gly Gln Pro Phe Tyr Ala Ala Phe Gly Asn Arg Pro Asn Asp | |
| 515 520 525 | |
| Val Phe Ala Tyr Arg Gln Val Gly Leu Pro Glu Ser Arg Ile Phe Thr | |
| 530 535 540 | |
| Val Asn Pro Arg Gly Glu Leu Ile Gln Glu Leu Ile Lys Asn His Lys | |
| 545 550 555 560 | |
| Ser Thr Tyr Gln Arg Leu Gly Glu Val Val Glu Leu Leu Phe Pro Pro | |
| 565 570 575 | |
| Val Ala Arg Gly Pro Ser Thr Asp Leu Ala Asn Pro Glu Tyr Ser Asn | |
| 580 585 590 | |
| Phe Cys Tyr Trp Arg Glu Pro Leu Pro Ala Val Asp Leu Asp Thr Leu | |
| 595 600 605 | |
| Asp | |

By assessing expression of mouse LIPIN3 mRNA from adipose tissue from lean versus obese BSB mice, overexpression of mouse LIPIN3 as indicated by mRNA expression has been associated with obesity, as shown in Example 4. Moreover, alterations of the human LIPIN3 gene (mutants) have been associated with obesity in human patients, as shown in Example 5.

GenBank Accession No. AL132654 discloses a 556 amino acid predicted protein encoded by some but not all of the LIPIN3 gene, Sanger Centre Accession No. CAC00516 discloses a 254 predicted amino acid sequence encoded by some but not all of the LIPIN3 gene, and Sanger Centre Accession No. CAC36284 discloses a 302 predicted amino acid sequence encoded by some but not all of the LIPIN3 gene.

Table 3 shows the predicted polypeptide sequence disclosed in GenBank Accession No AL132654. Table 4 shows the predicted amino acid sequence disclosed in Sanger Accession No. CAC00516. Table 5 shows the predicted amino acid sequence disclosed in Sanger Accession No. CAC36284. However, the GenBank and Sanger Accession Numbers provide only an incomplete characterization of the LIPIN3 gene. Specifically, the LIPIN3 coding region was incomplete, and amino acids 437-609 were omitted in the GenBank sequence, amino acids 1-135 and 437-609 were omitted in the Sanger CAC36284 sequence, and amino acids 136-609 were omitted in the Sanger CAC00516 sequence. The Sanger sequences also have additional amino acids sequences between positions 95 and 96 of the Lipin3 amino acid sequence shown in Table 2. Further, disclosure of elevated levels of LIPIN3 expression associated with a disease, namely obesity, was not made in the GenBank and Sanger Accession Numbers.

TABLE 3

Predicted amino acid sequence disclosed in GlenBank Accession
No AL132654.

| | |
|---|---|
| Met Asn Tyr Val Gly Gln Leu Ala Glu Thr Val Phe Gly Thr Val Lys | (SEQ ID NO:4) |
| 1 5 10 15 | |
| Glu Leu Tyr Arg Gly Leu Asn Pro Ala Thr Leu Ser Gly Gly Ile Asp | |
| 20 25 30 | |
| Val Leu Val Val Lys Gln Val Asp Gly Ser Phe Arg Cys Ser Pro Phe | |
| 35 40 45 | |
| His Val Arg Phe Gly Lys Leu Gly Val Leu Arg Ser Arg Glu Lys Val | |
| 50 55 60 | |
| Val Asp Ile Glu Leu Asn Gly Glu Pro Val Asp Leu His Met Lys Leu | |
| 65 70 75 80 | |
| Gly Asp Ser Gly Glu Ala Phe Phe Val Gln Glu Leu Glu Ser Asp Asp | |
| 85 90 95 | |
| Glu His Val Pro Pro Gly Leu Cys Thr Ser Pro Ile Pro Trp Gly Gly | |
| 100 105 110 | |
| Leu Ser Gly Phe Pro Ser Asp Ser Gln Leu Gly Thr Ala Ser Glu Pro | |
| 115 120 125 | |
| Glu Gly Leu Val Met Ala Gly Thr Ala Ser Thr Gly Arg Arg Lys Arg | |
| 130 135 140 | |
| Arg Arg Arg Arg Lys Pro Lys Gln Lys Glu Asp Ala Val Ala Thr Asp | |
| 145 150 155 160 | |
| Ser Ser Pro Glu Glu Leu Glu Ala Gly Ala Glu Ser Glu Leu Ser Leu | |
| 165 170 175 | |
| Pro Glu Lys Leu Arg Pro Glu Pro Pro Gly Ser Val Gln Leu Glu Glu | |
| 180 185 190 | |
| Lys Ser Ser Leu Gln Pro Lys Asp Ile Tyr Pro Tyr Ser Asp Gly Glu | |
| 195 200 205 | |
| Trp Pro Pro Gln Ala Ser Leu Ser Ala Gly Glu Leu Thr Ser Pro Lys | |
| 210 215 220 | |
| Ser Asp Ser Glu Leu Glu Val Arg Thr Pro Glu Pro Ser Pro Leu Arg | |
| 225 230 235 240 | |
| Ala Glu Ser His Met Gln Trp Ala Trp Gly Arg Leu Pro Lys Ser Pro | |
| 245 250 255 | |
| Lys Arg Ser Gln His Leu Gly Pro Ser Asp Ile Tyr Leu Asp Asp Leu | |
| 260 265 270 | |
| Pro Ser Leu Asp Ser Glu Asn Ala Ala Leu Tyr Phe Pro Gln Ser Asp | |
| 275 280 285 | |
| Ser Gly Leu Gly Ala Arg Arg Trp Ser Glu Pro Ser Ser Gln Lys Ser | |
| 290 295 300 | |
| Leu Arg Asp Pro Asn Pro Glu His Gln Pro Glu Pro Thr Leu Asp Thr | |
| 305 310 315 320 | |
| Val Asp Thr Ile Ala Leu Ser Leu Cys Gly Gly Leu Ala Asp Ser Arg | |
| 325 330 335 | |
| Asp Ile Ser Leu Glu Lys Phe Asn Gln His Ser Val Ser Tyr Gln Asp | |
| 340 345 350 | |
| Leu Thr Lys Asn Pro Gly Leu Leu Asp Asp Pro Asn Leu Val Val Lys | |
| 355 360 365 | |
| Ile Asn Gly Lys His Tyr Asn Trp Ala Val Ala Ala Pro Met Ile Leu | |
| 370 375 380 | |
| Ser Leu Gln Ala Phe Gln Lys Asn Leu Pro Lys Val Met Val Arg Glu | |
| 385 390 395 400 | |
| His His Gly Gln Ala Gly Glu Gly Glu Asp Ala Pro Glu Gly Trp Ala | |
| 405 410 415 | |
| Met Val Val Phe Leu Ala Thr Gln Gly Leu Pro Gly Arg Gly Gly Glu | |
| 420 425 430 | |
| Lys Thr Glu Val Leu Ser Ser Asp Asp Asp Ala Pro Asp Ser Pro Val | |
| 435 440 445 | |
| Ile Leu Glu Ile Pro Ser Leu Pro Pro Ser Thr Pro Pro Ser Thr Pro | |
| 450 455 460 | |
| Thr Tyr Lys Lys Ser Leu Arg Leu Ser Ser Asp Gln Ile Arg Arg Leu | |
| 465 470 475 480 | |
| Asn Leu Gln Glu Gly Ala Asn Asp Val Val Phe Ser Val Thr Thr Gln | |
| 485 490 495 | |
| Tyr Gln Gly Thr Cys Arg Cys Lys Ala Thr Ile Tyr Leu Trp Lys Trp | |
| 500 505 510 | |
| Asp Asp Lys Val Val Ile Ser Asp Ile Asp Gly Thr Ile Thr Lys Ser | |
| 515 520 525 | |
| Asp Ala Leu Gly His Ile Leu Pro Gln Leu Gly Lys Asp Trp Thr His | |
| 530 535 540 | |
| Gln Gly Ile Thr Ser Leu Tyr His Lys Ile Gln Leu | |

TABLE 4

Predicted amino acid sequence disclosed in Sanger Accession
No. CAC00516.

| | |
|---|---|
| Met Asn Tyr Val Gly Gln Leu Ala Glu Thr Val Phe Gly Thr Val Lys | (SEQ ID NO:5) |
| 1 5 10 15 | |
| Glu Leu Tyr Arg Gly Leu Asn Pro Ala Thr Leu Ser Gly Gly Ile Asp | |
| 20 25 30 | |
| Val Leu Val Val Lys Gln Val Asp Gly Ser Phe Arg Cys Ser Pro Phe | |
| 35 40 45 | |
| His Val Arg Phe Gly Lys Leu Gly Val Leu Arg Ser Arg Glu Lys Val | |
| 50 55 60 | |
| Val Asp Ile Glu Leu Asn Gly Glu Pro Val Asp Leu His Met Lys Leu | |
| 65 70 75 80 | |
| Gly Asp Ser Gly Glu Ala Phe Phe Val Gln Glu Leu Glu Ser Asp Asp | |
| 85 90 95 | |
| Glu His Val Pro Pro Gly Leu Cys Thr Ser Pro Ile Pro Trp Gly Gly | |
| 100 105 110 | |
| Leu Ser Gly Phe Pro Ser Asp Ser Gln Leu Gly Thr Ala Ser Glu Pro | |
| 115 120 125 | |
| Glu Gly Leu Val Met Ala Gly Thr Ala Ser Thr Gly Arg Arg Lys Arg | |
| 130 135 140 | |
| Arg Arg Arg Arg Lys Pro Lys Gln Lys Glu Asp Ala Val Ala Thr Asp | |
| 145 150 155 160 | |
| Ser Ser Pro Glu Glu Leu Glu Ala Gly Ala Glu Ser Glu Leu Ser Leu | |
| 165 170 175 | |
| Pro Glu Lys Leu Arg Pro Gln Pro Pro Gly Ser Val Gln Leu Glu Glu | |
| 180 185 190 | |
| Lys Ser Ser Leu Gln Pro Lys Asp Ile Tyr Pro Tyr Ser Asp Gly Glu | |
| 195 200 205 | |
| Trp Pro Pro Gln Ala Ser Leu Ser Ala Gly Glu Leu Thr Ser Pro Lys | |
| 210 215 220 | |
| Ser Asp Ser Glu Leu Glu Val Arg Thr Pro Glu Pro Ser Pro Leu Arg | |
| 225 230 235 240 | |
| Ala Glu Ser His Met Gln Trp Ala Trp Gly Arg Leu Pro Lys | |
| 245 250 | |

TABLE 5
Predicted amino acid sequence of Sanger CAC36284.

| Ser Pro Lys Arg Ser Gln His Leu Gly Pro Ser Asp Ile Tyr Leu Asp | (SEQ ID NO:6) |
|---|---|
| 1 5 10 15 | |
| Asp Leu Pro Ser Leu Asp Ser Glu Asn Ala Ala Leu Tyr Phe Pro Gln | |
| 20 25 30 | |
| Ser Asp Ser Gly Leu Gly Ala Arg Arg Trp Ser Glu Pro Ser Ser Gln | |
| 35 40 45 | |
| Lys Ser Leu Arg Asp Pro Asn Pro Glu His Glu Pro Glu Pro Thr Leu | |
| 50 55 60 | |
| Asp Thr Val Asp Thr Ile Ala Leu Ser Leu Cys Gly Gly Leu Ala Asp | |
| 65 70 75 80 | |
| Ser Arg Asp Ile Ser Leu Glu Lys Phe Asn Gln His Ser Val Ser Tyr | |
| 85 90 95 | |
| Gln Asp Leo Thr Lys Asn Pro Gly Leu Leu Asp Asp Pro Asn Leu Val | |
| 100 105 110 | |
| Val Lys Ile Asn Gly Lys His Tyr Asn Trp Ala Val Ala Ala Pro Met | |
| 115 120 125 | |
| Ile Leu Ser Leu Gln Ala Phe Gln Lys Asn Leu Pro Lys Val Met Val | |
| 130 135 140 | |
| Arg Glu His His Gly Gln Ala Gly Gln Gly Glu Asp Ala Pro Glu Gly | |
| 145 150 155 160 | |
| Trp Ala Met Val Val Phe Leu Ala Thr Gln Gly Leu Pro Gly Arg Gly | |
| 165 170 175 | |
| Gly Gln Lys Thr Glu Val Leu Ser Ser Asp Asp Asp Ala Pro Asp Ser | |
| 180 185 190 | |
| Pro Val Ile Leu Glu Ile Pro Ser Leu Pro Pro Ser Thr Pro Pro Ser | |
| 195 200 205 | |
| Thr Pro Thr Tyr Lys Lys Ser Leu Arg Leu Ser Ser Asp Gln Ile Arg | |
| 210 215 220 | |
| Arg Leu Asn Leu Gln Glu Gly Ala Asn Asp Val Val Phe Ser Val Thr | |
| 225 230 235 240 | |
| Thr Gln Tyr Gln Gly Thr Cys Arg Cys Lys Ala Thr Ile Tyr Leu Trp | |
| 245 250 255 | |
| Lys Trp Asp Asp Lys Val Val Ile Ser Asp Ile Asp Gly Thr Ile Thr | |
| 260 265 270 | |
| Lys Ser Asp Ala Leu Gly His Ile Leu Pro Gln Leu Gly Lys Asp Trp | |
| 275 280 285 | |
| Thr His Gln Gly Ile Thr Ser Leu Tyr His Lys Ile Gln Leu | |
| 290 295 300 | |

Reue et al., Nature Biotechnology 27:121-124 (2001) disclose an 86 amino acid and a 229 amino acid peptide corresponding to the Lipin-3 polypeptide sequence. See http://genetics.nature.com/supplementary_info/. Tables 6A and 6B shows the amino acid sequences disclosed in Reue et al. Reue et al. disclose only portions of the Lipin3 protein. Moreover, Reue et al. do not disclose an association of elevated levels of LIPIN3 expression and a disease. By contrast, the present inventors provide a more complete description and characterization of the LIPIN3 coding region. Specifically, the present inventors identified a 609 amino acid Lipin3 polypeptide. The present inventors further demonstrated that overexpression of LIPIN3 mRNA is associated with obesity (shown in Example 4), and that alterations in the LIPIN3 gene (mutants) correlate with obesity (Example 5).

TABLES 6A and 6B
Amino acid sequences disclosed in Reue et al. supra.

6A: NLIP domain

| | |
|---|---|
| Leu Asn Pro Ala Thr Leu Ser Gly Gly Ile Asp Val Leu Val Val Lys | (SEQ ID NO:7 and SEQ ID NO:8) |
| 1 5 10 15 | |
| Gln Val Asp Gly Ser Phe Arg Cys Ser Pro Phe His Val Arg Phe Gly | |
| 20 25 30 | |
| Lys Leu Gly Val Leu Arg Ser Arg Glu Lys Val Val Asp Ile Glu Leu | |
| 35 40 45 | |
| Asn Gly Glu Pro Val Asp Leu His Met Lys Leu Gly Asp Ser Gly Glu | |
| 50 55 60 | |
| Ala Phe Phe Val Gln Glu Leu Glu Ser Asp Asp Glu His Val Pro Pro | |
| 65 70 75 80 | |
| Gly Leu Cys Thr Ser Pro Ile | |
| 85 | |

6B: CLIP domain

```
Tyr Lys Lys Ser Leu Arg Leu Ser Ser Asp Gln Ile Arg Arg Leu Asn
  1 5 10 15
Leu Gln Glu Gly Ala Asn Asp Val Val Phe Ser Val Thr Thr Gln Tyr
                        20 25 30
Gln Gly Thr Cys Arg Cys Lys Ala Thr Ile Tyr Leu Trp Lys Trp Asp
                35 40 45
Asp Lys Val Val Ile Ser Asp Ile Asp Gly Thr Ile Thr Lys Ser Asp
        50 55 60
Ala Leu Gly His Ile Leu Pro Gln Leu Gly Lys Asp Trp Thr His Gln
65 70 75 80
Gly Ile Thr Ser Leu Tyr His Lys Ile Gln Leu Asn Gly Tyr Lys Phe
                                85 90 95
Leu Tyr Cys Ser Ala Arg Ala Ile Gly Met Ala Asp Leu Thr Lys Gly
                        100 105 110
Tyr Leu Gln Trp Val Ser Glu Gly Gly Cys Ser Leu Pro Lys Gly Pro
                115 120 125
Ile Leu Leu Ser Pro Ser Ser Leu Phe Ser Ala Leu His Arg Glu Val
        130 135 140
Ile Glu Lys Lys Pro Glu Val Phe Lys Val Ala Cys Leu Ser Asp Ile
145 150 155 160
Gln Gln Leu Phe Leu Pro His Gly Gln Pro Phe Tyr Ala Ala Phe Gly
                                165 170 175
Asn Arg Pro Asn Asp Val Phe Ala Tyr Arg Gln Val Gly Leu Pro Glu
                        180 185 190
Ser Arg Ile Phe Thr Val Asn Pro Arg Gly Glu Leu Ile Gln Glu Leu
                195 200 205
Ile Lys Asn His Lys Ser Thr Tyr Glu Arg Leu Gly Glu Val Val Glu
        210 215 220
Leu Leu Phe Pro Pro Val
225 230
```

After the identification of the LIPIN3 and Lipin3 sequences discussed herein, the Celera database disclosed a predicted LIPIN3 polynucleotide and polypeptide sequences, shown in Table 7A and 7B, respectively. The Celera sequence contains additional amino acid(s) inserted between positions 63 and 64, between positions 95 and 96, positions 134 and 135 and positions 311 and 312 of the Lipin3 amino acid sequence shown in Table 2. The Celera database does not disclose an association of elevated levels of LIPIN3 expression and a disease. By contrast, the present inventors demonstrated that overexpression of LIPIN3 mRNA is associated with obesity (shown in Example 4), and that alterations in the LIPIN3 gene (mutants) correlate with obesity (Example 5).

TABLE 7A
Amino acid sequence disclosed in the Celera database.

| Met Asn Tyr Val Gly Gln Leu Ala Glu Thr Val Phe Gly Thr Val Lys | (SEQ ID NO:9) |
|---|---|
| 1 5 10 15 | |
| Glu Leu Tyr Arg Gly Leu Asn Pro Ala Thr Leu Ser Gly Gly Ile Asp | |
| 20 25 30 | |
| Val Leu Val Val Lys Gln Val Asp Gly Ser Phe Arg Cys Ser Pro Phe | |
| 35 40 45 | |
| His Val Arg Phe Gly Lys Leu Gly Val Leu Arg Ser Arg Glu Lys Val | |
| 50 55 60 | |
| Val Asp Ile Glu Leu Asn Gly Glu Pro Val Asp Leu His Met Lys Leu | |
| 65 70 75 80 | |
| Gly Asp Ser Gly Glu Ala Phe Phe Val Gln Glu Leu Glu Ser Asp Asp | |
| 85 90 95 | |
| Glu His Val Pro Pro Gly Leu Cys Thr Ser Pro Ile Pro Trp Gly Gly | |
| 100 105 110 | |
| Leu Ser Gly Phe Pro Ser Asp Ser Gln Leu Gly Thr Ala Ser Glu Pro | |
| 115 120 125 | |
| Glu Gly Leu Val Met Ala Gly Thr Ala Ser Thr Gly Arg Arg Lys Arg | |
| 130 135 140 | |
| Arg Arg Arg Arg Lys Pro Lys Gln Lys Glu Asp Ala Val Ala Thr Asp | |
| 145 150 155 160 | |
| Ser Ser Pro Glu Glu Leu Glu Ala Gly Ala Glu Ser Glu Leu Ser Leu | |
| 165 170 175 | |
| Pro Glu Lys Leu Arg Pro Glu Pro Pro Gly Ser Val Gln Leu Glu Glu | |
| 180 185 190 | |
| Lys Ser Ser Leu Gln Pro Lys Asp Ile Tyr Pro Tyr Ser Asp Gly Glu | |
| 195 200 205 | |
| Trp Pro Pro Gln Ala Ser Leu Ser Ala Gly Glu Leu Thr Ser Pro Lys | |
| 210 215 220 | |
| Ser Asp Ser Glu Leu Glu Val Arg Thr Pro Glu Pro Ser Pro Leu Arg | |
| 225 230 235 240 | |
| Ala Glu Ser His Met Gln Trp Ala Trp Gly Arg Leu Pro Lys Gln Thr | |
| 245 250 255 | |
| Glu Ala Gly Ala Asp Leu Gln Pro Asp Thr Glu Asp Pro Thr Leu Val | |
| 260 265 270 | |
| Gly Pro Pro Leu His Thr Pro Glu Thr Glu Glu Ser Lys Thr Gln Ser | |
| 275 280 285 | |
| Ser Gly Asp Met Gly Leu Pro Pro Ala Ser Lys Ser Trp Ser Trp Ala | |
| 290 295 300 | |
| Thr Leu Glu Val Pro Val Pro Thr Gly Gln Pto Gln Arg Val Ser Arg | |
| 305 310 315 320 | |
| Gly Lys Gly Ser Pro Lys Arg Ser Gln His Leu dy Pro Ser Asp Ile | |
| 325 330 335 | |
| Tyr Leu Asp Asp Leu Pro Ser Leu Asp Ser Glu Asn Ala Ala Leu Tyr | |
| 340 345 350 | |
| Phe Pro Gln Ser Asp Ser Gly Leu Gly Ala Arg Arg Trp Ser Glu Pro | |
| 355 360 365 | |
| Ser Ser Gln Lys Ser Leu Arg Asp Pro Asn Pro Glu His Gln Pro Glu | |
| 370 375 380 | |
| Pro Thr Leu Asp Thr Val Asp Thr Ile Ala Leu Ser Leu Cys Gly Gly | |
| 385 390 395 400 | |
| Leu Ala Asp Ser Arg Asp Ile Ser Leu Glu Lys Phe Asn Gln His Ser | |
| 405 410 415 | |
| Val Ser Tyr Gln Asp Leu Thr Lys Asn Pro Gly Leu Leu Asp Asp Pro | |
| 420 425 430 | |
| Asn Leu Val Val Lys Ile Asn Gly Lys His Tyr Asn Trp Ala Val Ala | |
| 435 440 445 | |
| Ala Pro Met Ile Leu Ser Leu Gln Ala Phe Gln Lys Asn Leu Pro Lys | |
| 450 455 460 | |
| Ser Thr Met Asp Lys Leu Glu Arg Glu Lys Met Pro Arg Lys Gly Gly | |
| 465 470 475 480 | |
| Arg Trp Trp Phe Ser Trp Arg Arg Arg Asp Phe Leu Ala Glu Glu Arg | |
| 485 490 495 | |
| Ser Ala Gln Lys Glu Lys Thr Ala Ala Lys Glu Gln Gln Gly Glu Lys | |
| 500 505 510 | |
| Thr Glu Val Leu Ser Ser Asp Asp Asp Ala Pro Asp Ser Pro Val Ile | |
| 515 520 525 | |
| Leu Glu Ile Pro Ser Leu Pro Pro Ser Thr Pro Pro Ser Thr Pro Thr | |
| 530 535 540 | |
| Tyr Lys Lys Ser Leu Arg Leu Ser Ser Asp Gln Ile Arg Arg Leu Asn | |
| 545 550 555 560 | |
| Leu Gln Glu Gly Ala Asn Asp Val Val Phe Ser Val Thr Thr Gln Tyr | |
| 565 570 575 | |
| Gln Gly Thr Cys Arg Cys Lys Ala Thr Ile Tyr Leu Trp Lys Trp Asp | |
| 580 585 590 | |
| Asp Lys Val Val Ile Ser Asp Ile Asp Gly Thr Ile Thr Lys Ser Asp | |
| 595 600 605 | |
| Ala Leu Gly His Ile Leu Pro Gln Leu Gly Lys Asp Trp Thr His Gln | |
| 610 615 620 | |
| Gly Ile Thr Ser Leu Tyr His Lys Ile Gln Leu Asn Gly Tyr Lys Phe | |
| 625 630 635 640 | |
| Leu Tyr Cys Ser Ala Arg Ala Ile Gly Met Ala Asp Leu Thr Lys Gly | |
| 645 650 655 | |
| Tyr Leu Gln Trp Val Ser Glu Gly Gly Cys Ser Leu Pro Lys Gly Pro | |
| 660 665 670 | |
| Ile Leu Leu Ser Pro Ser Ser Leu Phe Ser Ala Leu His Arg Glu Val | |
| 675 680 685 | |
| Ile Glu Lys Lys Pro Glu Val Phe Lys Val Ala Cys Leu Ser Asp Ile | |
| 690 695 700 | |
| Gln Gln Leu Phe Leu Pro His Gly Gln Pro Phe Tyr Ala Ala Phe Gly | |
| 705 710 715 720 | |
| Asn Arg Pro Asn Asp Val Phe Ala Tyr Arg Gln Val Gly Leu Pro Glu | |
| 725 730 735 | |
| Ser Arg Ile Phe Thr Val Asn Pro Arg Gly Glu Leu Ile Gln Glu Leu | |
| 740 745 750 | |
| Ile Lys Asn His Lys Ser Thr Tyr Glu Arg Leu Gly Glu Val Val Glu | |
| 755 760 765 | |
| Leu Leu Phe Pro Pro Val Ala Arg Gly Pro Ser Thr Asp Leu Ala Asn | |
| 770 775 780 | |
| Pro Glu Tyr Ser Asn Phe Cys Tyr Trp Arg Glu Pro Leu Pro Ala Val | |
| 785 790 795 800 | |
| Asp Leu Asp Thr Leu Asp | |
| 805 | |

TABLE 7B
Polynucleotide sequence disclosed in the Celera database.

(SEQ ID NO:10)

| | |
|---|---|
| atgaactacg tggggcagct ggcggagacg gtgtttggga cggtgaagga gctgtaccgg | 60 |
| ggcctgaacc cagccacact gagcggcggc attgacgtgc tggtggtgaa gaggtggacg | 120 |
| gctcgttccg gtgctcaccc ttccacgtgc gttttggcaa gctgggcgtc ctgcggtcgc | 180 |
| gggagaaggt ggtagacatt gagctcaatg gggagcctgt ggacttgcac tgaagcttgg | 240 |
| ggacagcggg gaggccttct ttgttcagga gctggagagc gatgatgaaa tgtgcctccc | 300 |
| ggcctgtgca cctcacccat cccttggggg ggtctgtctg gcttcccctc ggactcccag | 360 |
| ctgggcactg ccagtgagcc tgagggcctc gtcatggcag gcacggcctc cactgggcgg | 420 |
| aggaagaggc gtcgcaggag gaaacccaag cagaaagagg atgcaggcaa ctgattctag | 480 |
| tccagaggaa ctggaggcag gcgctgagag tgagctatcc ctgccgaaaa gctgaggcca | 540 |
| gagcccccag gcagtgtcca gttggaagag aagtcttcac tgcagccaaa gacatctacc | 600 |
| cctactcgga tggcgagtgg cccccccagg ccagcctctc agcaggtgag ctaacatccc | 660 |
| ctaagagcga ctcggagctg gaggtgcgga ccccggagcc cagtccctaa gagccgagtc | 720 |
| ccacatgcag tgggcctggg ggaggctgcc taagcaaaca gagctggtgc cgaccttcag | 780 |
| cctgacacag aggatcccac tctagtgggt ccccctctcc acaceccaga gacagaggaa | 840 |
| agcaagactc agagctctgg ggacatgggc ctccctcctg ccccaagtca tggagctggg | 900 |
| ccactctgga ggttccagtt cccaccgggc agccagagag ggtctccagg gggaaaggct | 960 |
| ccccaaagag aagccagcac ctgggcccca gtgacatcta ctggatgact tgccctccct | 1020 |
| ggactctgag aatgcagcgc tttacttccc ccaaagtgac tctgggctgg gggccagaag | 1080 |
| atggagtgaa cccagcagtc agaagtccct gagggacccc accctgaaca tgaacctgaa | 1140 |
| cccactctgg acacagtgga tacaatagca ctgtccctct gtggtggact ggctgacagc | 1200 |
| cgggacatct ccctagagaa attcaaccag cacagcgtct cttaccagga cctcaccaaa | 1260 |
| aaccccggac ttttggatga cccaaaccta gtggtgaaaa tcaatggaaa gcattataac | 1320 |
| tgggctgtgg ctgcccccat gatcctctcc ctgcaagcct tccagaaaaa cttgcccaag | 1380 |
| agcaccatgg acaagctgga gagggagaag atgccccgga gggtgggcga tggtggtttt | 1440 |
| cctggcgacg cagggacttc ctggccgagg agcgcagtgc ccagaaggag aagactgcag | 1500 |
| ccaaggagca gcagggggag aagacagaag tcctgaagtg atgacgatgc cccagacagc | 1560 |
| cctgtgatcc tggagatccc ctccttgcca ccctccactc cacectacac tcctacctac | 1620 |
| aagaagtccc tccgcctctc ctccgatcag atccggcgcc tgaacctgca agaaggtgcc | 1680 |
| aatgatgtgg tcttcagcgt gaccactcag taccagggca cctgccgctg caaggccacc | 1740 |
| atctacctgt ggaaatggga cgacaaggtg gtcatctctg acatcgacgg caccatcacc | 1800 |
| aagtcagatg ctctgggcca tatcctgccc cagctgggga aagactggac acaccagggc | 1860 |
| atcaccagtc tctatcacaa aatccaacta aatgggacaa gttcctgtac tgctcggcgc | 1920 |
| gggccattgg catggcggac ctcaccaagg ggtacctgca gtgggtgagc gaggggggct | 1980 |
| gtagcctccc caagggcccc atccttctgt ctcccagcag cctcttctct gccctccaca | 2040 |
| gagaggtgat cgagaagaaa ccagaggtgt tcaaggtcgc ctgcctgagt gacatccagc | 2100 |
| agctgtttct gccccacgga cagcccttct atgctgcctt tgggaatagg cccaatgatg | 2160 |
| tctttgccta ccggcaggtg ggcctgcctg agtcacgcat cttcacagtc aacccccggg | 2220 |
| gagagctcat ccaggagctc ataaagaacc acaaatccac gtatgagcgg cttggtgaag | 2280 |
| tggtcgagct cctcttccca cctgtggccc gtggccccag cacagacctg gccaaccctg | 2340 |
| aatacagtaa cttctgctac tggcgggagc cactgcctgc tgtggacctt gataccctgg | 2400 |
| actga 2405 | |

Accordingly, the present invention provides an isolated polynucleotide that comprises (a) a polynucleotide encoding the polypeptide having the sequence shown in Table 9, or its complement; or (b) a polynucleotide encoding amino acids 588-609 shown in Table 9, or its complement. The present invention also provides an isolated polynucleotide that comprises a polynucleotide encoding a region of the polypeptide shown in Table 9 or Table 7A, wherein said region is at least about 5 contiguous amino acids in length. For example, the invention includes an isolated polynucleotide encoding at least 10, at least 15, at least 20, at least 25, or more contiguous amino acids of a sequence depicted in Table 9 or Table 7A.

In another embodiment, the invention provides an isolated polynucleotide comprising a polynucleotide encoding a region of at least 5 contiguous amino acids of the polypeptide shown in Table 9, said region comprising amino acids 438 and 439 shown in Table 9. In yet another embodiment, the invention provides an isolated polynucleotide comprising a polynucleotide encoding a region of at least 5 contiguous amino acids of the peptide shown in Table 9, said region comprising amino acids 587 and 588 shown in Table 9.

In another embodiment, the invention provides an isolated polynucleotide comprising a polynucleotide encoding a region of at least about 5 contiguous amino acids of the polypeptide sequence shown in Table 9, said region comprising amino acids 21 and 22 shown in Table 9. In another embodiment, the invention provides an isolated polynucleotide comprising a polynucleotide encoding a region of at least about 5 contiguous amino acids of the polypeptide sequence shown in Table 9, said region comprising amino acids 95 and 96 shown in Table 9. In another embodiment, the invention provides an isolated polynucleotide comprising a polynucleotide encoding a region of at least about 5 contiguous amino acids of the polypeptide sequence shown in Table 9, said region comprising amino acids 356 and 357 shown in Table 9. In another embodiment, the invention provides an isolated polynucleotide comprising a polynucleotide encoding a region of at least about 5 contiguous amino acids of the polypeptide sequence shown in Table 9, said region comprising amino acids 438 and 439 shown in Table 9. For example, the invention includes an isolated polynucleotide encoding at least 10, at least 15, at least 20, at least 25 contiguous amino acids of a sequence depicted in Table 9 or Table 7A.

The present invention further provides an isolated polynucleotide that comprises a polynucleotide having the sequence shown in Table 1, or its complement. The invention also provides an isolated polynucleotide that comprises a region of at least 10 contiguous polynucleotides shown in Table 1. An embodiment of the invention includes, for example, an isolated polynucleotide encoding a region of at least 15, at least 18, at least 20, at least 25, at least 30, at least 50, and at least 100 or more contiguous polynucleotides.

In another embodiment, the invention provides a region of the polynucleotide shown in Table 1 wherein said region does not correspond identically in its entirety to any other known polynucleotide. In some embodiments, the invention provides a polynucleotide comprising a region of the polynucleotide shown in Table 1 wherein said region does not correspond identically in its entirety to the mouse Lpn3 polynucleotide. The polynucleotide sequence of the mouse Lpn3 polynucleotide is available in GenBank.

Portions of the LIPIN3 polynucleotide sequence which are not in the LIPIN1 or LIPIN2 polynucleotide sequence are desirable for specifically detecting LIPIN3. Sequences that are common to LIPIN1, LIPIN2 and LIPIN3 are useful for assessing expression of all three gene simultaneously (or individual levels of expression of all three genes, if expression is assessed using, for example, an array). The polynucleotide sequences of LIPIN1 and LIPIN2 are available in GenBank as Accession Nos. XM028744 and XM041136, respectively. It is routine to compare the polynucleotide sequences and identify further sequences that are unique to LIPIN3 or sequences that are shared between LIPIN3 and LIPIN1 and/or LIPIN2.

Another embodiment of the invention provides an isolated polynucleotide having at least 85%, at least 88%, at least 90%, at least 92%, at least 94%, at least 96%, at least 98%, or at least 99% sequence identity with the polynucleotides of the invention as described herein. One embodiment provides an isolated polynucleotide having at least 85%, at least 88%, at least 90%, at least 92%, at least 94%, at least 96%, at least 98%, or at least 99% sequence identity with the sequence shown in Table 1. In other embodiments, isolated polynucleotides additionally have less than 85%, 83%, 80%, 75%, 70% sequence identity with the sequence of mouse LIPIN3 (available in GenBank Accession number NM022883). The invention also includes isolated polynucleotides having at least 85%, at least 88%, at least 90%, at least 92%, at least 94%, at least 96%, at least 98%, or at least 99% sequence identity to fragments of at least 10 contiguous nucleotides (or more, such as 15, 18, 20, 25, 35, 40, 45, 50, 60, 75, or 100 contiguous nucleotides) of the sequence shown in Table 1.

In terms of hybridization conditions, the higher the sequence identity required, the more stringent are the hybridization conditions if such sequences are determined by their ability to hybridize to a polynucleotide sequence of the invention. Accordingly, the invention also includes polynucleotides that are able to hybridize to a sequence comprising a polynucleotide of the invention as discussed herein. In one embodiment, the invention provides an isolated polynucleotide comprising at least 10 contiguous nucleotides (or more, such as at least 15, 18, 20, 25, 35, 40, 45, 50, 60, 75 or 100 contiguous nucleotides) of a polynucleotide of the invention. The hybridization conditions would be stringent, i.e., 80° C. (or higher temperature) and 6×SSC (or less concentrated SSC). For discussion regarding hybridization reactions, see below.

In one embodiment, the invention provides an isolated polynucleotide comprising a sequence of at least 10 contiguous nucleotides (or more, such as 15, 18, 20, 25, 35, 40, 45, 50, 60, 75, or 100 (or more) contiguous nucleotides) that hybridizes with a polynucleotide (such as DNA or RNA) comprising the sequence depicted in Table 1 or Table 7B, or fragments thereof as described above, under conditions where it does not hybridize with other polynucleotides from a mammalian cell, preferably a human cell, or under conditions in which hybridization to a polynucleotide having the sequence depicted in Table 1 or Table 7B is enriched relative to hybridization with other polynucleotides from a mammalian cell.

These embodiments are particularly useful in the diagnostic (detection) context.

In another embodiment, the invention includes a polynucleotide sequence comprising at least 10, preferably 15, preferably 18, preferably 20, more preferably 25, more preferably 35, more preferably 50, still more preferably 75 contiguous nucleotides of the non-coding (i.e., flanking) or intron regions shown in FIG. 1. These embodiments may be particularly useful as diagnostic probes, or as primers for amplification of LIPIN3 introns, intro-exon boundaries, or noncoding portions of the LIPIN3 gene.

It is understood that (unless otherwise specified or required), any embodiment of the invention described herein that is a polynucleotide encompasses both the double-stranded form and each of two complementary single-stranded forms known or predicted to make up the double stranded form.

Hybridization reactions can be performed under conditions of different “stringency”. Conditions that increase stringency of a hybridization reaction of widely known and published in the art. See, for example, Sambrook et al. (1989). Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water.

Probes from more than one polynucleotide sequence provided herein can hybridize with the same nucleic acid if the cDNA from which they were derived corresponds to one mRNA. By using probes, particularly labeled probes of DNA sequences, one can isolate homologous or related genes. The source of homologous genes can be any species, e.g. primate species, canines, felines, bovines, ovines, equines, yeast, nematodes. Probes of more than 10 nucleotides (“nt”) can be used, e.g. probes of a size within a range of about 15 nt, 18 nt, 20, nt, 25 nt, 75 nt, or 100 nt, but in general about 15 nt represents sufficient sequence for unique identification.

“Tₘ” is the temperature in degrees Centigrade at which 50% of a polynucleotide duplex made of complementary strands hydrogen bonded in anti-parallel direction by Watson-Crick base pairing dissociates into single strands under conditions of the experiment. Tₘ may be predicted according to a standard formula, such as:

Tₘ=81.5+16.6 log[X⁺]+0.41 (%G/C)−0.61 (%F)−600/L

where [X⁺] is the cation concentration (usually sodium ion, Na⁺) in mol/L; (%G/C) is the number of G and C residues as a percentage of total residues in the duplex; (%F) is the percent formamide in solution (wt/vol); and L is the number of nucleotides in each strand of the duplex.

The invention includes modifications to the LIPIN3 polynucleotides described above such as deletions, substitutions, additions, or changes in the nature of any nucleic acid moieties. A “modification” is any difference in nucleotide sequence as compared to a polynucleotide shown herein to encode a Lipin3 polypeptide, and/or any difference in terms of the nucleic acid moieties of the polynucleotide(s). Such changes can be useful to facilitate cloning and modifying expression of LIPIN3 polynucleotides. Such changes also can be useful for conferring desirable properties to the polynucleotide(s), such as stability. The definition of polynucleotide provided herein gives examples of these modifications. Hence, the invention also includes functionally-preserved variants of the nucleic acid sequences disclosed herein, which include nucleic acid substitutions, additions, and/or deletions. Variants include naturally occurring variants of the polynucleotide sequence (e.g. degenerate variants, allelic variants, etc. In general, allelic variants contain 15-25% base pair (bp) mismatches and can contain as little as 5-15%, or 2-5%, or 1-2% bp mismatch, as well as a single bp mismatch.

The invention encompasses LIPIN3 polynucleotides including full-length (unprocessed), processed, coding, non-coding or portions thereof. A partial map of the LIPIN3 genomic region is shown in FIG. 1, including predicted intron-exon boundaries for 15 exons. The invention can further include the 3′ and 5′ untranslated regions found in the mature mRNA, specific transcriptional and translational regulatory sequences, such as promoters, enhancers, etc., including about 1 kb, and possibly more of flanking genomic DNA at either the 5′ or 3′ end of the transcribed region. The genomic DNA can be isolated as a fragment of 100 kbp or smaller, and substantially free of flanking chromosomal sequence. The genomic DNA flanking the coding region, either 3′ or 5′, or internal regulatory sequences as sometime found in introns, contains sequences required for proper tissue, stage-specific or disease-state specific expression. Also embodied are the mRNA and cDNA sequences and fragments thereof, including fragments that include a portion of a Lipin3 encoding segment. Normally, mRNA species have contiguous exons, with the intervening introns, when present, being removed by nuclear RNA splicing, to create a continuous open reading frame encoding a polypeptide. mRNA species can also exist with both exons and introns, where the introns may be removed by alternative splicing. Furthermore, different species of mRNAs encoded by the same genomic species can exist at varying levels in a cell, and detection of these various levels of mRNA species can be indicative of differential expression of the encoded gene product in the cell.

The invention also encompasses polynucleotides encoding for functionally equivalent variants and derivatives of full-length Lipin3 and functionally equivalent fragments thereof which may enhance, decrease or not significantly affect properties of the polypeptides encoded thereby, provided that these functionally equivalent variants do not have the same amino acid sequence as in mouse Lipin3. For instance, changes in a DNA sequence that do not change the encoded amino acid sequence, as well as those that result in conservative substitutions of amino acid residues, non-deleterious non-conservative substitutions, one or a few amino acid deletions or additions, and substitution of amino acid residues by amino acid analogs are those which will not significantly affect properties of the encoded polypeptide. Nucleotide substitutions that do not alter the amino acid residues encoded can be useful for optimizing gene expression in different systems. Suitable substitutions are known to those of skill in the art and are made, for instance, to reflect preferred codon usage in the particular expression systems. In another example, alternatively spliced polynucleotides can give rise to a functionally equivalent fragment or variant of Lipin3. Alternatively processed polynucleotide sequence variants are defined as polynucleotide sequences corresponding to mRNAs that differ in sequence for one another but are derived from the same genomic region, for example, mRNAs that result from: 1) the use of alternative promoters; 2) the use of alternative polyadenylation sites; or 3) the use of alternative splice sites.

Mutant LIPIN3 Polynucleotides

The identification of an mutant LIPIN3 polynucleotide (allele) that correlates with obesity in a population of obese women is described in Example 5. This polymorphism is designated 11-33 (Gln634His), and results in a substitution at nucleotide position 1904. The polynucleotide sequence of the 11-33 allele is shown in Table 8.

TABLE 8

Position of LIPIN3 allele 11-33 (Gln634His) on the predicted
Celera LIPIN3 polynucleotide sequence.

(SEQ ID NO:11)

| | |
|---|---|
| atgaactacg tggggcagct ggcggagacg gtgtttggga cggtgaagga gctgtaccgg | 60 |
| ggcctgaacc cagccacact gagcggcggc attgacgtgc tggtggtgaa gaggtggacg | 120 |
| gctcgttccg gtgctcaccc ttccacgtgc gttttggcaa gctgggcgtc ctgcggtcgc | 180 |
| gggagaaggt ggtagacatt gagctcaatg gggagcctgt ggacttgcac tgaagcttgg | 240 |
| ggacagcggg gaggccttct ttgttcagga gctggagagc gatgatgaaa tgtgcctccc | 300 |
| ggcctgtgca cctcacccat cccttggggg ggtctgtctg gcttcccctc ggactcccag | 360 |
| ctgggcagtg ccagtgagcc tgagggcctc gtcatggcag gcacggcctc cactgggcgg | 420 |
| aggaagaggc gtcgcaggag gaaacccaag cagaaagagg atgcaggcaa ctgattctag | 480 |
| tccagaggaa ctggaggcag gcgctgagag tgagctatcc ctgccgaaaa gctgaggcca | 540 |
| gagcccccag gcagtgtcca gttggaagag aagtcttcac tgcagccaaa gacatctacc | 600 |
| cctactcgga tggcgagtgg cccccccagg ccagcctctc agcaggtgag ctaacatccc | 660 |
| ctaagagcga ctcggagctg gaggtgcgga ccccggagcc cagtccctaa gagccgagtc | 720 |
| ccacatgcag tgggcctggg ggaggctgcc taagcaaaca gagctggtgc cgaccttcag | 780 |
| cctgacacag aggatcccac tctagtgggt ccccctctcc acaccccaga gacagaggaa | 840 |
| agcaagactc agagctctgg ggacatgggc ctccctcctg ccccaagtca tggagctggg | 900 |
| ccactctgga ggttccagtt cccaccgggc agccagagag ggtctccagg gggaaaggct | 960 |
| ccccaaagag aagccagcac ctgggcccca gtgacatcta ctggatgact tgccctccct | 1020 |
| ggactctgag aatgcagcgc tttacttccc ccaaagtgac tctgggctgg gggccagaag | 1080 |
| atggagtgaa cccagcagtc agaagtccct gagggacccc accctgaaca tgaacctgaa | 1140 |
| cccactctgg acacagtgga tacaatagca ctgtccctct gtggtggact ggctgacagc | 1200 |
| cgggacatct ccctagagaa attcaaccag cacagcgtct cttaccagga cctcaccaaa | 1260 |
| aaccccggac ttttggatga cccaaaccta gtggtgaaaa tcaatggaaa gcattataac | 1320 |
| tgggctgtgg etgcccccat gatcctctcc ctgcaagcct tccagaaaaa cttgcceaag | 1380 |
| agcaccatgg acaagctgga gagggagaag atgccccgga gggtgggcga tggtggtttt | 1440 |
| cctggcgacg cagggacttc ctggccgagg agcgcagtgc ccagaaggag aagactgcag | 1500 |
| ccaaggagca gcagggggag aagacagaag tcctgaagtg atgacgatgc occagacaga | 1560 |
| cctgtgatcc tggagatccc ctccttgcca ccctccactc caccctccac tcctacctac | 1620 |
| aagaagtccc tccgcctctc ctccgatcag atccggcgcc tgaacctgca agaaggtgcc | 1680 |
| aatgatgtgg tcttcagcgt gaccactcag taccagggca cctgccgctg caaggccacc | 1740 |
| atctacctgt ggaaatggga cgacaaggtg gtcatctctg acatcgacgg caccatcacc | 1800 |
| aagtcagatg ctctgggcca tatcctgccc cagctgggga aagactggac acaccagggc | 1860 |
| atcaccagtc tctatcacaa aatccaacta aatgggacaa gttactgtac tgctcggcgc | 1920 |
| gggccattgg catggcggac ctcaccaagg ggtacctgca gtgggtgagc gaggggggct | 1980 |
| gtagcctccc caagggcccc atccttctgt ctcccagcag cctcttctct gccctccaca | 2040 |
| gagaggtgat cgagaagaaa ccagaggtgt tcaaggtcgc ctgcctgagt gacatccagc | 2100 |
| agctgtttct gccccacgga cagcccttct atgctgcctt tgggaatagg cccaatgatg | 2160 |
| tctttgccta ccggcaggtg ggcctgcctg agtcacgcat cttcacagtc aacccccggg | 2220 |
| gagagctcat ccaggagctc ataaagaacc acaaatccac gtatgagcgg cttggtgaag | 2280 |
| tggtcgagct cctcttccca cctgtggccc gtggccccag cacagacctg gccaaccctg | 2340 |
| aatacagtaa cttctgctac tggcgggagc cactgcctgc tgtggacctt gataccctgg | 2400 |
| actga 2405 | |

The mutant nucleotide is shown in bold capital letter.

The identification of four other polymorphisms found in a population of obese women is described in Example 5, depicted in FIG. 1, and further shown in Table 13.

A mutant LIPIN3 polynucleotide, or fragments thereof, may be useful in identifying predisposition to obesity or obesity-related diseases in an individual possessing an altered LIPIN3 gene, as described herein. Mutant LIPIN3 polynucleotides may also useful for diagnosing obesity or an obesity-related disease, as described herein.

Accordingly, the invention provides an isolated polynucleotide comprising the sequence shown in Table 8. The invention also provides a isolated polynucleotide comprising a region of the polynucleotide shown in Table 8. Thus, the invention provides an isolated polynucleotide comprising a region of at least about 10 contiguous nucleotides (or more, such as at least 15, 18, 20, 25, 35, 40, 45, 50, 60, 75 or 100 contiguous nucleotides), said region comprising nucleotide 1904 shown in Table 8.

In another embodiment, the invention provides an isolated polynucleotide having at least 85%, at least 88%, at least 90%, at least 92%, at least 94%, at least 96%, at least 98%, or at least 99% sequence identity with the sequence shown in Table 8, wherein the polynucleotide comprises a region of at least about 10 contiguous nucleotides (or more, such as at least 15, 18, 20, 25, 35, 40, 45, 50, 60, 75 or 100 contiguous nucleotides), said region comprising nucleotide 1904 shown in Table 8. In other embodiments, isolated polynucleotides additionally have less than about 85%, about 83%, about 80%, about 75%, about 70% sequence identity with the sequence of mouse LIPIN3. In another embodiment, the invention provides an isolated polynucleotide that specifically hybridizes to the polynucleotide shown in Table 8, or a region of at least about 10 contiguous nucleotides (or more, such as at least 15, 18, 20, 25, 35, 40, 45, 50, 60, 75 or 100 contiguous nucleotides), said region comprising nucleotide 1904 shown in Table 8.

In other embodiments, the invention comprises polynucleotides as described herein comprising the other polymorphisms shown in FIG. 1 and Table 13.

In other embodiments, the polynucleotide further comprises detectable label. Detectable labels are known in the art and include, for example, fluorescent dye label, biotin label, or radioactive label. In another embodiment, the polynucleotide is immobilized on a surface (an array), as described herein.

The invention also provides compositions comprising a vector(s) containing a LIPIN3 polynucleotide as well as compositions comprising a host cell containing a LIPIN3 polynucleotide, as described herein. When these compositions are to be used pharmaceutically, they are combined with a pharmaceutically acceptable excipient. Examples of pharmaceutically acceptable excipients are known in the art. When these compositions are to be used for detection, they are combined with a suitable substance such as a buffer, and they contain an amount effective to allow detection.

Preparation of LIPIN3 Polynucleotides

The polynucleotides of this invention can be obtained using chemical synthesis, recombinant methods, or PCR.

Methods of chemical polynucleotide synthesis are well known in the art and need not be described in detail herein. One of skill in the art can use the sequences provided herein and a commercial DNA synthesizer to produce a desired DNA sequence.

For preparing LIPIN3 polynucleotides using recombinant methods, a polynucleotide comprising a desired sequence can be inserted into a suitable vector, and the vector in turn can be introduced into a suitable host cell for replication and amplification. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al. (1989).

Alternatively, PCR allows reproduction of DNA sequences. PCR technology is well known in the art and is described in U.S. Pat. No. 4,683,195, 4,800,159, 4,754,065 and 4,683,202, as well as PCR: The Polymerase Chain Reaction, Mullis et al. eds., Birkauswer Press, Boston (1994).

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., (1989), for example.

If used as a vaccine (i.e., pharmaceutical composition for eliciting an immune response), plasmids containing LIPIN3 polynucleotides are preferably prepared as described by Horn et al. ((1995) Human Gene Therapy 6:565-573) which produces a pharmaceutical grade plasmid DNA suitable for administration.

Probes or primers specific to the polynucleotides described herein can be generated using the polynucleotide sequences disclosed herein. The probes or primers are preferably at least about a 10 nt, 12 nt, 15 nt, 16 nt, 18 nt, 20 nt, 22 nt, 24 nt, or 25 nt fragment of a corresponding contiguous sequence any one of the polynucleotide sequences provided herein, and can be less than 2 kb, 1 kb, 0.5 kb, 0.1 kb, or 0.05 kb in length. The probes or primers can be synthesized chemically or can be generated from longer polynucleotides using restriction enzymes. The probes or primers can be labeled, for example, with a radioactive, biotinylated, or fluorescent tag or immobilized on a solid support. Preferably, probes or primers are designed based upon an identifying sequence of any one of the polynucleotide sequences provided herein. More preferably, probes or primers are designed based on a contiguous sequence of one of the subject polynucleotides that remain unmasked following application of a masking program for masking low complexity (e.g., XBLAST) to the sequence, i.e., one would select an unmasked region, as indicated by the polynucleotides outside the poly-n stretches of the masked sequence produced by the masking program.

Cloning and Expression Vectors Comprising a LIPIN3 Polynucleotide

The present invention further includes a variety of vectors (i.e., cloning and expression vectors) having cloned therein LIPIN3 polynucleotide(s). These vectors can be used for expression of recombinant polypeptides as well as a source of LIPIN3 polynucleotides. Cloning vectors can be used to obtain replicate copies of the LIPIN3 polynucleotides they contain, or as a means of storing the polynucleotides in a depository for future recovery. Expression vectors (and host cells containing these expression vectors) can be used to obtain polypeptides produced from the polynucleotides they contain. They may also be used where it is desirable to express Lipin3 polypeptides in an individual, such as for eliciting an immune response via the polypeptide(s) encoded in the expression vector(s). Suitable cloning and expression vectors include any known in the art e.g., those for use in bacterial, mammalian, yeast and insect expression systems. Specific vectors and suitable host cells are known in the art and need not be described in detail herein. For example, see Gacesa and Ramji, Vectors, John Wiley & Sons (1994).

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins substances, e.g., ampicillin, neomycyin, methotrexate, etc.; (b) complement auxotrophic deficiencies; or (c) supply critical nutrients not available from complex media. The choice of the proper marker gene will depend on the host cell, and appropriate genes for different hosts are known in the art. Cloning and expression vectors also typically contain a replication system recognized by the host.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide encoding a Lipin3 polypeptide of interest. The polynucleotide encoding the Lipin3 polypeptide is operatively linked to suitable transcriptional controlling elements, such as promoters, enhancers and terminators. For expression (i.e., translation), one or more translational controlling elements are also usually required, such as ribosome binding sites, translation initiation sites, and stop codons. These controlling elements (transcriptional and translational) may be derived from LIPIN3 polynucleotides (i.e., the LIPIN3 gene), or they may be heterologous (i.e., derived from other genes and/or other organisms). A polynucleotide sequence encoding a signal peptide can also be included to allow a Lipin3 polypeptide to cross and/or lodge in cell membranes or be secreted from the cell. A number of expression vectors suitable for expression in eukaryotic cells including yeast, avian, and mammalian cells are known in the art.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (where the vector is an infectious agent, such as vaccinia virus). The choice of means of introducing vectors or LIPIN3 polynucleotides will often depend on the host cell.

Host Cells Transformed with LIPIN3 Polynucleotides

Another embodiment of this invention are host cells transformed with (i.e., comprising) LIPIN3 polynucleotides and/or vectors having LIPIN3 polynucleotide(s) sequences, as described above. Both prokaryotic and eukaryotic host cells may be used. Prokaryotic hosts include bacterial cells, for example E. coli, B. subtilis and mycobacteria. E. coli cells are particularly useful for producing Lipin3 polypeptides. Komachi et al. (1994) Genes Dev. 8: 2857-2867. Among eukaryotic hosts are yeast, insect, avian, plant and mammalian cells. Host systems are known in the art and need not be described in detail herein.

The host cells of this invention can be used, inter alia, as repositories of LIPIN3 polynucleotides and/or vehicles for production of LIPIN3 polynucleotides and/or polypeptides. Host cells can also serve as repositories of mutant LIPIN3 polynucleotides, as further described herein. Such hosts cells can be useful for screening, as further described herein.

Arrays Comprising LIPIN3 Polynucleotides

Polynucleotide arrays provide a high throughput technique that can assay a large number of polynucleotides or polypeptides in a sample. This technology can be used as a tool to test for differential expression. A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Alternatively, the polynucleotides of the test sample can be immobilized on the array, and the probes detectably labeled. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

Arrays can be used to examine differential expression of genes and can be used to determine gene function. For example, arrays can be used to detect differential expression of a gene corresponding to a polynucleotide described herein, where expression is compared between a test cell and control cell. For example, high expression of a particular message in an cell from a subject who is obese or has an obesity-related disease, which is not observed in a corresponding normal cell, can indicate a obesity specific gene product. Exemplary uses of arrays are further described in, for example, Pappalarado et al., Sem. Radiation Oncol. (1998) 8:217; and Ramsay Nature Biotechnol. (1998) 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

A gene or polynucleotide that is differentially expressed in a cell when the polynucleotide is detected at higher or lower levels in a cell of an obese individual compared with a cell of the same cell type that is from an individual who is not obese. Typically, screening for polynucleotides differentially expressed focuses on a polynucleotide that is expressed such that, for example, mRNA is found at levels at least about 25%, at least about 50% to about 75%, at least about 90%, at least about 2-fold, at least about 4-fold; at least about 5-fold, at least about 10-fold, or at least about 50-fold or more, higher (e.g. overexpressed) or lower (e.g., underexpressed) in a cell from an individual who is obese or has an obesity-related disease when compared with a cell of the same cell type that is not from such an individual. The comparison can be made between two tissues, for example, if one is using in situ hybridization or another assay method that allows some degree of discrimination among cell types in the tissue. The comparison may also be made between cells removed from their tissue source. Thus, a polypeptide encoded by a polynucleotide that is differentially expressed in a cell from an obese individual would be of clinical significance with respect to obesity and obesity-related diseases.

Thus, the invention provides an array comprising at least two LIPIN3 polynucleotides as described herein. In another embodiment, the invention provides an array comprising at least two regions of a polynucleotide comprising the sequence shown in Table 1, wherein said region is at least 10 contiguous nucleotides (or more, such as at least 15, 18, 20, 25, 35, 40, 45, 50, 60, 75 or 100 contiguous nucleotides). In another embodiment, the array further comprises at least one polynucleotide, or a region thereof, selected from the group consisting of a LIPIN1 polynucleotide, a LIPIN2 polynucleotide, a polynucleotide shown in Table 3, a polynucleotide shown in Table 4, and a polynucleotide shown in Table 5, a polynucleotide shown in Table 6, a polynucleotide shown in Table 7B.

Arrays are also useful for detecting mutant LIPIN3 polynucleotides. Mutant LIPIN3 polynucleotides can be detected in genomic DNA, e.g., genomic DNA isolated from the blood of an individual or from another tissue sample. Mutant LIPIN3 polynucleotides can also be detected using cDNA or mRNA from an individual possessing an altered LIPIN3 polynucleotide, if the mutant LIPIN3 polynucleotide results in an mRNA that is altered in size (for example). A mutant LIPIN3 gene may also result in the differential expression (increased or decreased) of a LIPIN3 mRNA, which can be detected as described herein.

In one preferred embodiment of the present invention, an array comprises one or more isolated polynucleotides that specifically hybridize to the polynucleotide shown in Table 8, or to a region of at least about 10 contiguous nucleotides (or more, such as at least 15, 18, 20, 25, 35, 40, 45, 50, 60, 75 or 100 or more contiguous nucleotides) of the polynucleotide shown in Table 8, said region comprising nucleotide 1904 shown in Table 8. In another embodiment, the array further comprises at least one polynucleotide, or a region thereof, selected from the group consisting of a LIPIN1 polynucleotide, a LIPIN2 polynucleotide, a LIPIN3 polynucleotide as described herein, a polynucleotide shown in Table 3, a polynucleotide shown in Table 4, a polypeptide shown in Table 5, a polynucleotide shown in Table 6, and a polynucleotide shown in Table 7. In other embodiments, the array comprises one or more polynucleotide comprising a mutation described in Table 13 (and shown in FIG. 1).

“Reference sequences” or “reference polynucleotides” as used herein in the context of differential gene expression analysis and diagnosis/prognosis refers to a selected set of polynucleotides, which selected set includes at least one or more of the differentially expressed polynucleotides described herein. A plurality of reference sequences, preferably comprising positive and negative control sequences, can be included as reference sequences. Additional suitable reference sequences are found in GenBank, Unigene, and other nucleotide sequence databases (including, e.g., expressed sequence tag (EST), partial, and full-length sequences).

“Reference array” means an array having reference sequences for use in hybridization with a sample, where the reference sequences include all, at least one of, or any subset of the differentially expressed polynucleotides described herein. Usually such an array will include at least 2 different reference sequences, and can include any one or all of the provided differentially expressed sequences. Arrays of interest can further comprise sequences, including polymorphisms, of other genetic sequences, particularly other sequences of interest for screening for a disease or disorder (e.g., obesity, diabetes, hypolipideamia, hyperlipidemia, or other related or unrelated diseases, disorders, or conditions). The oligonucleotide sequence on the array will usually be at least about 12 nt in length, and can be of about the length of the provided sequences, or can extend into the flanking regions to generate fragments of 100 nt to 200 nt in length or more. Reference arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

A “reference expression pattern” or “REP” as used herein refers to the relative levels of expression of a selected set of genes, particularly of differentially expressed genes, that is associated with a selected cell type, e.g., a normal cell, a cell from an obese individual, a cell exposed to an environmental stimulus, and the like. A “test expression pattern” or “TEP” refers to relative levels of expression of a selected set of genes, particularly of differentially expressed genes, in a test sample (e.g., a cell of unknown or suspected disease state, from which mRNA is isolated).

REPs can be generated in a variety of ways according to methods well known in the art. For example, REPs can be generated by hybridizing a control sample to an array having a selected set of polynucleotides (particularly a selected set of differentially expressed polynucleotides), acquiring the hybridization data from the array, and storing the data in a format that allows for ready comparison of the REP with a TEP. Alternatively, all expressed sequences in a control sample can be isolated and sequenced, e.g., by isolating mRNA from a control sample, converting the mRNA into cDNA, and sequencing the cDNA. The resulting sequence information roughly or precisely reflects the identity and relative number of expressed sequences in the sample. The sequence information can then be stored in a format (e.g., a computer-readable format) that allows for ready comparison of the REP with a TEP. The REP can be normalized prior to or after data storage, and/or can be processed to selectively remove sequences of expressed genes that are of less interest or that might complicate analysis (e.g., some or all of the sequences associated with housekeeping genes can be eliminated from REP data).

TEPs can be generated in a manner similar to REPs, e.g., by hybridizing a test sample to an array having a selected set of polynucleotides, particularly a selected set of differentially expressed polynucleotides, acquiring the hybridization data from the array, and storing the data in a format that allows for ready comparison of the TEP with a REP. The REP and TEP to be used in a comparison can be generated simultaneously, or the TEP can be compared to previously generated and stored REPs.

In one embodiment of the invention, comparison of a TEP with a REP involves hybridizing a test sample with an array, where the reference array has one or more reference sequences for use in hybridization with a sample. The reference sequences include all, at least one of, or any subset of the differentially expressed polynucleotides described herein. Hybridization data for the test sample is acquired, the data normalized, and the produced TEP compared with a REP generated using an array having the same or similar selected set of differentially expressed polynucleotides. Probes that correspond to sequences differentially expressed between the two samples will show decreased or increased hybridization efficiency for one of the samples relative to the other.

Methods for collection of data from hybridization of samples with a reference arrays are well known in the art. For example, the polynucleotides of the reference and test samples can be generated using a detectable fluorescent label, and hybridization of the polynucleotides in the samples detected by scanning the microarrays for the presence of the detectable label using, for example, a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample (e.g., a test sample) is compared to the fluorescent signal from another sample (e.g., a reference sample), and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

In general, the test sample is classified as having a gene expression profile corresponding to that associated with a disease or non-disease state by comparing the TEP generated from the test sample to one or more REPs generated from reference samples (e.g., from samples associated obesity or obesity-related diseases, samples affected by a disease other than obesity, normal samples, etc.). The criteria for a match or a substantial match between a TEP and a REP include expression of the same or substantially the same set of reference genes, as well as expression of these reference genes at substantially the same levels (e.g., no significant difference between the samples for a signal associated with a selected reference sequence after normalization of the samples, or at least no greater than about 25% to about 40% difference in signal strength for a given reference sequence. In general, a pattern match between a TEP and a REP includes a match in expression, preferably a match in qualitative or quantitative expression level, of at least one of, all or any subset of the differentially expressed genes of the invention.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

Uses for and Methods Using LIPIN3 Polynucleotides

The polynucleotides of this invention have several uses. LIPIN3 polynucleotides are useful, for example, as probes for screening for differential expression, e.g., overexpression of LIPIN3 mRNA, which has been associated with obesity.

Mutant LIPIN3 polynucleotides are useful genetic markers for diagnosing a predisposition to obesity or an obesity-related disease and for diagnosing obesity or an obesity-related disease. Diagnostic (i.e., detection) and screening methods are described in more detail below.

LIPIN3 polynucleotides, as well as their gene products, are of particular interest as genetic or biochemical markers (e.g., in blood or tissues) that could detect propensity to obesity, the signs of obesity or an obesity related disease, and/or to monitor the efficacy of various therapies and preventative interventions. Diagnostic (i.e., detection) and screening methods are described in more detail below.

LIPIN3 polynucleotides are also useful in expression systems for the recombinant production of LIPIN3 or LIPIN3 fragments. They are also useful as hybridization probes to assay for the presence of LIPIN3 polynucleotide (or related) sequences in a sample using methods well known to those in the art. Further, LIPIN3 polynucleotides are also useful as primers to effect amplification of desired polynucleotides, such as in a polymerase chain reaction (PCR). PCR has been described above. For example, primers can be designed to effect amplification of a region of LIPIN3 that is altered in a LIPIN3 mutant that correlates with obesity (mutant allele). A plurality of primers can be designed to test for the presence of altered LIPIN3 polynucleotides as described in further detail in Example 5. The conditions for carrying out PCR reactions depend on the specificity desired, which in turn can be adjusted by the primer used and the reaction conditions. Such adjustments are known in the art and need not be discussed in detail herein.

The LIPIN3 polynucleotides of this invention can be used in expression systems to produce Lipin3 polypeptides or recombinant forms of Lipin3 polypeptides, which have enhanced, equivalent, or different, desirable properties. These recombinant forms are made by using routine methods in the art. Examples of recombinant forms of Lipin3 polypeptides include, but are not limited to, fusion proteins. Fusion proteins may also facilitate purification.

LIPIN3 polynucleotide may be used to obtain other genes and gene products involved in a LIPIN3 pathway, as described below.

Polypeptides of the Invention

The present invention encompasses human Lipin3 polypeptide sequences. Identification of Lipin3 polypeptide sequence is described in Example 1, and the Lipin3 polypeptide sequence is shown in Table 9.

TABLE 9
LIP1N-3 amino acid sequence (609 amino acids).

| | |
|---|---|
| Met Asn Tyr Val Gly Gln Leu Ala Glu Thr Val Phe Gly Thr Val Lys | (SEQ ID NO:12) |
| 1 5 10 15 | |
| Glu Leu Tyr Arg Gly Leu Asn Pro Ala Thr Leu Ser Gly Gly Ile Asp | |
| 20 25 30 | |
| Val Leu Val Val Lys Gln Val Asp Gly Ser Phe Arg Cys Ser Pro Phe | |
| 35 40 45 | |
| His Val Arg Phe Gly Lys Leu Gly Val Leu Arg Ser Arg Glu Lys Val | |
| 50 55 60 | |
| Asp Ile Glu Leu Asn Gly Gln Pro Val Asp Leu His Met Lys Leu Gly | |
| 65 70 75 80 | |
| Asp Ser Gly Glu Ala Phe Phe Val Gln Glu Leu Gln Ser Asp Asp Leu | |
| 85 90 95 | |
| Ser Ala Gly Glu Leu Thr Ser Pro Lys Ser Asp Ser Glu Leu Glu Val | |
| 100 105 110 | |
| Arg Thr Pro Glu Pro Ser Pro Leu Arg Ala Glu Ser His Met Gln Trp | |
| 115 120 125 | |
| Ala Trp Gly Arg Leu Pro Lys Gly Ser Pro Lys Arg Ser Gln His Leu | |
| 130 135 140 | |
| Gly Pro Ser Asp Ile Tyr Leu Asp Asp Leu Pro Ser Leu Asp Ser Glu | |
| 145 150 155 | |
| Asn Ala Ala Leu Tyr Phe Pro Gln Ser Asp Ser Gly Leu Gly Ala Arg | |
| 165 170 175 | |
| Arg Trp Ser Glu Pro Ser Ser Gln Lys Ser Leu Arg Asp Pro Asn Pro | |
| 180 185 190 | |
| Glu His Glu Pro Glu Pro Thr Leu Asp Thr Val Asp Thr Ile Ala Leu | |
| 195 200 205 | |
| Ser Leu Cys Gly Gly Leu Ala Asp Ser Arg Asp Ile Ser Leu Glu Lys | |
| 210 215 220 | |
| Phe Asn Gln His Ser Val Ser Tyr Gln Asp Leu Thr Lys Asn Pro Gly | |
| 225 230 235 240 | |
| Leu Leu Asp Asp Pro Asa Leu Val Val Lys Ile Asn Gly Lys His Tyr | |
| 245 250 255 | |
| Asn Trp Ala Val Ala Ala Pro Met Ile Leu Ser Leu Gln Ala Phe Gln | |
| 260 265 270 | |
| Lys Asn Leu Pro Lys Val Met Val Arg Glu His His Gly Gln Ala Gly | |
| 275 280 285 | |
| Glu Gly Gln Asp Ala Pro Glu Gly Trp Ala Met Val Val Phe Leu Ala | |
| 290 295 300 | |
| Thr Gln Gly Leu Pro Gly Arg Gly Gly Glu Lys Thr Glu Val Leu Ser | |
| 305 310 315 320 | |
| Ser Asp Asp Asp Ala Pro Asp Ser Pro Val Ile Leu Gln Ile Pro Ser | |
| 325 330 335 | |
| Leu Pro Pro Ser Thr Pro Pro Ser Thr Pro Thr Tyr Lys Lys Ser Leu | |
| 340 345 350 | |
| Arg Leu Ser Ser Asp Gln Ile Arg Arg Leu Asn Leu Gln Gln Gly Ala | |
| 355 360 365 | |
| Asn Asp Val Val Phe Ser Val Thr Thr Gln Tyr Gln Gly Thr Cys Arg | |
| 370 375 380 | |
| Cys Lys Ala Thr Ile Tyr Leu Trp Lys Trp Asp Asp Lys Val Val Ile | |
| 385 390 395 400 | |
| Ser Asp Ile Asp Gly Thr Ile Thr Lys Ser Asp Ala Leu Gly His Ile | |
| 405 410 415 | |
| Leu Pro Gln Leu Gly Lys Asp Trp Thr His Gln Gly Ile Thr Ser Leu | |
| 420 425 430 | |
| Tyr His Lys Ile Gln Leu Asn Gly Tyr Lys Phe Leu Tyr Cys Ser Ala | |
| 435 440 445 | |
| Arg Ala Ile Gly Met Ala Asp Leu Thr Lys Gly Tyr Leu Gln Trp Val | |
| 450 455 460 | |
| Ser Glu Gly Gly Cys Ser Leu Pro Lys Gly Pro Ile Leu Leu Ser Pro | |
| 465 470 475 480 | |
| Ser Ser Leu Phe Ser Ala Leu His Arg Glu Val Ile Glu Lys Lys Pro | |
| 485 490 495 | |
| Glu Val Phe Lys Val Ala Cys Leu Ser Asp Ile Gln Gln Leu Phe Leu | |
| 500 505 510 | |
| Pro His Gly Gln Pro Phe Tyr Ala Ala Phe Gly Asn Arg Pro Asn Asp | |
| 515 520 525 | |
| Val Phe Ala Tyr Arg Gln Val Gly Leu Pro Glu Ser Arg Ile Phe Thr | |
| 530 535 540 | |
| Val Asn Pro Arg Gly Glu Leu Ile Gln Glu Leu Ile Lys Aso His Lys | |
| 545 550 555 560 | |
| Ser Thr Tyr Glu Arg Leu Gly Glu Val Val Glu Leu Leu Phe Pro Pro | |
| 565 570 575 | |
| Val Ala Arg Gly Pro Ser Thr Asp Leu Ala Asn Pro Glu Tyr Ser Asn | |
| 580 585 590 | |
| Phe Cys Tyr Trp Arg Glu Pro Leu Pro Ala Val Asp Leu Asp Thr Leu | |
| 595 600 605 | |
| Asp | |

The polypeptides may comprise any novel region (i.e., not disclosed in the public domain as of the filing date of the original application of this series) of Table 9. Unless specifically stated, the term “polypeptide(s)” shall include all polypeptide embodiments of this invention.

As noted above and shown in Tables 2-6, GenBank Accession No. AL132654 discloses a 556 amino acid predicted protein encoded by some but not all of the LIPIN3 gene, Sanger Centre Accession No. CAC00516 discloses a 254 predicted amino acid sequence encoded by some but not all of the LIPIN3 gene, Sanger Centre Accession No. CAC36284 discloses a 302 predicted amino acid sequence encoded by some but not all of the LIPIN3 gene, and Reue et al., Nature Biotechnology 27:121-124 (2001) disclose an 86 amino acid and a 229 amino acid peptide corresponding to the Lipin-3 polypeptide sequence. In addition, after the invention of the Lipin3 polypeptide disclosed herein, Celera disclosed a predicted Lipin3 polypeptide of 806 amino acids (shown in Table 7A).

The Lipin3 polypeptides of the invention have a variety of uses. The polypeptides are of particular interest as genetic or biochemical markers (e.g., in blood or tissues) that indicate propensity to obesity, the signs of obesity or an obesity related disease, and/or to monitor the efficacy of various therapies and preventative interventions. Diagnostic (i.e., detection) and screening methods are described in more detail below. The polypeptides of the invention also are of use in making antibodies that bind to these polypeptides, their use as an agent to screen pharmaceutical candidates (both in vitro and in vivo), their use in rational (i.e., structure-based) drug design, as well as other possible therapeutic uses (for example, if full-length Lipin3 exerts its action by binding to another protein, a Lipin3 polypeptide that binds competitively to Lipin3 could compromise LIPIN3 function as a competitive inhibitor and thus exert therapeutic activity). The Lipin3 polypeptides may also be used to identifying proteins especially those from humans that bind (or interact physically) with Lipin3 which could thus themselves be drug targets.

The size of the Lipin3 polypeptide fragments may vary widely. Thus, the invention includes polypeptide fragments of full-length Lipin3 comprising a portion of the amino acid sequence depicted in Table 2 in which the Lipin3 polypeptide is at least about 5, about 10, about 15, 25, 50, 75, 100, 150, or more amino acids in length. As is evident to one skilled in the art, these Lipin3 polypeptides, regardless of their size, may also be associated with, or conjugated with, other substances or agents to facilitate, enhance, or modulate function and/or specificity of a Lipin3 polypeptide.

Therefore, the invention provides an isolated polypeptide comprising a polypeptide encoded by any of the polynucleotides of the invention, as described herein. In another embodiment, the invention provides an isolated polypeptide comprising (a) a polypeptide having the sequence shown in Table 9; or (b) amino acids 588-609 shown in Table 9. The present invention also provides an isolated polypeptide that comprises a region (fragment) of the polypeptide shown in Table 9 or Table 7A, wherein said region is at least about 5 contiguous amino acids in length. For example, the invention includes at least about 5, about 10, about 15, 25, 50, 75, 100, 150, or more contiguous amino acids of a sequence depicted in Table 9 or Table 7A.

In another embodiment, the invention provides an isolated polypeptide comprising a region of at least 5 contiguous amino acids of the polypeptide shown in Table 9, said region comprising amino acids 438 and 439 shown in Table 9. In yet another embodiment, the invention provides an isolated polypeptide comprising a region of at least 5 contiguous amino acids of the peptide shown in Table 9, said region comprising amino acids 587 and 588 shown in Table 9.

In another embodiment, the invention provides a polypeptide comprising a region of at least about 5 (or about 10, about 15, 25, 50, 75, 100, 150, or more) contiguous amino acids of the polypeptide sequence shown in Table 9, said region comprising amino acids 21 and 22 shown in Table 9. In another embodiment, the invention provides a polypeptide comprising a region of at least about 5 (or about 10, about 15, 25, 50, 75, 100, 150, or more) contiguous amino acids of the polypeptide sequence shown in Table 9, said region comprising amino acids 95 and 96 shown in Table 9. In another embodiment, the invention provides a polypeptide comprising a region of at least about 5 (or about 10, about 15, 25, 50, 75, 100, 150, or more) contiguous amino acids of the polypeptide sequence shown in Table 9, said region comprising amino acids 356 and 357 shown in Table 9.

In other embodiment, the Lipin3 polypeptide are different from the mouse Lipin3 polypeptides (as disclosed in GenBank Accession No. NM 02283). In still other embodiments, the Lipin3 polypeptides are different from the human Lipin1 and human Lipin2 polypeptides (shown in Genbank Accession Nos. XM028744 and 041136, respectively).

In another aspect, the invention provides mutant Lipin3 polypeptides wherein the altered Lipin3 polypeptide comprises a genetic or biochemical markers (e.g., in blood or tissues) that will indicate propensity to obesity or an obesity related disease, the signs of obesity or an obesity related disease, aid in the diagnosis of obesity or an obesity related disease, and/or to monitor the efficacy of various therapies and preventative interventions. As noted above, the identification of a mutant LIPIN3 polynucleotide (allele) that correlates with obesity in a population of obese women is described in Example 5. This polymorphism is designated 11-33 (Gln634His), and results in a substitution at nucleotide position 1904 and a corresponding substitution of a His for Gln at position 634 of the Celera amino acid sequence (and at position 421 of the Lipin3 sequence shown in Table 9). The amino acid sequence of the 11-33 allele is shown in Table 10.

TABLE 10

Position of LIPIN3 allele 11-33 (Gln634His) on the predicted
Celera LIPIN3 polypeptide sequence.

| | |
|---|---|
| Met Asn Tyr Val Gly Gln Leu Ala Glu Thr Val Phe Gly Thr Val Lys | (SEQ ID NO:13) |
| 1 5 10 15 | |
| Glu Leu Tyr Arg Gly Leu Asn Pro Ala Thr Leu Ser Gly Gly Ile Asp | |
| 20 25 30 | |
| Val Leu Val Val Lys Gln Val Asp Gly Ser Phe Arg Cys Ser Pro Phe | |
| 35 40 45 | |
| His Val Arg Phe Gly Lys Leu Gly Val Leu Arg Ser Arg Glu Lys Val | |
| 50 55 60 | |
| Val Asp Ile Glu Leu Asn Gly Glu Pro Val Asp Leu His Met Lys Leu | |
| 65 70 75 80 | |
| Gly Asp Ser Gly Glu Ala Phe Phe Val Gln Glu Leu Glu Ser Asp Asp | |
| 85 90 95 | |
| Glu His Val Pro Pro Gly Leu Cys Thr Ser Pro Ile Pro Trp Gly Gly | |
| 100 105 110 | |
| Leu Ser Gly Phe Pro Ser Asp Ser Gln Leu Gly Thr Ala Ser Glu Pro | |
| 115 120 125 | |
| Glu Gly Leu Val Met Ala Gly Thr Ala Ser Thr Gly Arg Arg Lys Arg | |
| 130 135 140 | |
| Arg Arg Arg Arg Lys Pro Lys Gln Lys Glu Asp Ala Val Ala Thr Asp | |
| 145 150 155 160 | |
| Ser Ser Pro Glu Glu Leu Glu Ala Gly Ala Glu Ser Glu Leu Ser Leu | |
| 165 170 175 | |
| Pro Glu Lys Leu Arg Pro Glu Pro Pro Gly Ser Val Gln Leu Glu Glu | |
| 180 185 190 | |
| Lys Ser Ser Leu Gln Pro Lys Asp Ile Tyr Pro Tyr Ser Asp Gly Glu | |
| 195 200 205 | |
| Trp Pro Pro Gln Ala Ser Leu Ser Ala Gly Glu Leu Thr Ser Pro Lys | |
| 210 215 220 | |
| Ser Asp Ser Glu Leu Glu Val Arg Thr Pro Glu Pro Ser Pro Leu Arg | |
| 225 230 235 240 | |
| Ala Glu Ser His Met Gln Trp Ala Trp Gly Arg Leu Pro Lys Gln Thr | |
| 245 250 255 | |
| Glu Ala Gly Ala Asp Leu Gln Pro Asp Thr Glu Asp Pro Thr Leu Val | |
| 260 265 270 | |
| Gly Pro Pro Leu His Thr Pro Glu Thr Glu Glu Ser Lys Thr Gln Ser | |
| 275 280 285 | |
| Ser Gly Asp Met Gly Leu Pro Pro Ala Ser Lys Ser Trp Ser Trp Ala | |
| 290 295 300 | |
| Thr Leu Glu Val Pro Val Pro Thr Gly Gln Pro Glu Arg Val Ser Arg | |
| 305 310 315 320 | |
| Gly Lys Gly Ser Pro Lys Arg Ser Gln His Leu Gly Pro Ser Asp Ile | |
| 325 330 335 | |
| Tyr Leu Asp Asp Leu Pro Ser Leu Asp Ser Glu Asn Ala Ala Leu Tyr | |
| 340 345 350 | |
| Phe Pro Gln Ser Asp Ser Gly Leu Gly Ala Arg Arg Trp Ser Glu Pro | |
| 355 360 365 | |
| Ser Ser Gln Lys Ser Leu Arg Asp Pro Asn Pro Glu His Glu Pro Glu | |
| 370 375 380 | |
| Pro Thr Leu Asp Thr Val Asp Thr Ile Ala Leu Ser Leu Cys Gly Gly | |
| 385 390 395 400 | |
| Leu Ala Asp Ser Arg Asp Ile Ser Leu Glu Lys Phe Asn Gln His Ser | |
| 405 410 415 | |
| Val Ser Tyr Gln Asp Leu Thr Lys Asn Pro Gly Leu Leu Asp Asp Pro | |
| 420 425 430 | |
| Asn Leu Val Val Lys Ile Asn Gly Lys His Tyr Asn Trp Ala Val Ala | |
| 435 440 445 | |
| Ala Pro Met Ile Leu Ser Leu Gln Ala Phe Gln Lys Asn Leu Pro Lys | |
| 450 455 460 | |
| Ser Thr Met Asp Lys Leu Glu Arg Glu Lys Met Pro Arg Lys Gly Gly | |
| 465 470 475 480 | |
| Arg Trp Trp Phe Ser Trp Arg Arg Arg Asp Phe Leu Ala Glu Glu Arg | |
| 485 490 495 | |
| Ser Ala Gln Lys Glu Lys Thr Ala Ala Lys Glu Gln Gln Gly Glu Lys | |
| 500 505 510 | |
| Thr Glu Val Leu Ser Ser Asp Asp Asp Ala Pro Asp Ser Pro Val Ile | |
| 515 520 525 | |
| Leu Glu Ile Pro Ser Leu Pro Pro Ser Thr Pro Pro Ser Thr Pro Thr | |
| 530 535 540 | |
| Tyr Lys Lys Ser Leu Arg Leu Ser Ser Asp Gln Ile Arg Arg Leu Asn | |
| 545 550 555 560 | |
| Leu Gln Glu Gly Ala Asn Asp Val Val Phe Ser Val Thr Thr Gln Tyr | |
| 565 570 575 | |
| Gln Gly Thr Cys Arg Cys Lys Ala Thr Ile Tyr Leu Trp Lys Trp Asp | |
| 580 585 590 | |
| Asp Lys Val Val Ile Ser Asp Ile Asp Gly Thr Ile Thr Lys Ser Asp | |
| 595 600 605 | |
| Ala Leu Gly His Ile Leu Pro Gln Leu Gly Lys Asp Trp Thr His Gln | |
| 610 615 620 | |
| Gly Ile Thr Ser Leu Tyr His Lys Ile HIS Leu Asn Gly Tyr Lys Phe | |
| 625 630 635 640 | |
| Leu Tyr Cys Ser Ala Arg Ala Ile Gly Met Ala Asp Leu Thr Lys Gly | |
| 645 650 655 | |
| Tyr Leu Gln Trp Val Ser Glu Gly Gly Cys Ser Leu Pro Lys Gly Pro | |
| 660 665 670 | |
| Ile Leu Leu Ser Pro Ser Ser Leu Phe Ser Ala Leu His Arg Glu Val | |
| 675 680 685 | |
| Ile Glu Lys Lys Pro Glu Val Phe Lys Val Ala Cys Leu Ser Asp Ile | |
| 690 695 700 | |
| Gln Gln Leu Phe Leu Pro His Gly Gln Pro Phe Tyr Ala Ala Phe Gly | |
| 705 710 715 720 | |
| Asn Arg Pro Asn Asp Val Phe Ala Tyr Arg Gln Val Gly Leu Pro Glu | |
| 725 730 735 | |
| Ser Arg Ile Phe Thr Val Asn Pro Arg Gly Glu Leu Ile Gln Glu Leu | |
| 740 745 750 | |
| Ile Lys Asn His Lys Ser Thr Tyr Glu Arg Leu Gly Glu Val Val Glu | |
| 755 760 765 | |
| Leu Leu Phe Pro Pro Val Ala Arg Gly Pro Ser Thr Asp Leu Ala Asn | |
| 770 775 780 | |
| Pro Glu Tyr Ser Asn Phe Cys Tyr Trp Arg Glu Pro Leu Pro Ala Val | |
| 785 790 795 800 | |
| Asp Leu Asp Thr Leu Asp | |
| 805 | |

The mutant amino acid is shown in bold capital letter.

In one embodiment, the isolated polypeptide comprises a polypeptide having the amino acid sequence shown in Table 10, or a polynucleotide comprising a region consisting at least about 5 contiguous amino acids (or more, e.g. about 10, about 15, 25, 50, 75, 100, 150, amino acids in length) of the amino acid sequence shown in Table 10 wherein said region includes amino acid 634.

In another aspect, the polypeptide of the invention includes an epitope. By epitope, reference is meant to an antigenic determinant of a polypeptide. The presence of an epitope is demonstrated by the ability of an antibody to bind a polypeptide. Two antibodies are considered to be directed to the same epitope if they cross block each others binding to the same polypeptide.

In another aspect, the polypeptide of the invention is immobilized on a solid support, e.g. an array. Methods of making and using arrays are known in the art and described herein.

The invention includes modifications to Lipin3 polypeptides including functionally equivalent fragments of the Lipin3 polypeptides which do not significantly affect their properties and variants which have enhanced or decreased activity, provided that these sequences are different from that of M. musculus Lipin3. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tryosine. These polypeptides also include glycosylated and nonglycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the variable region. Changes in the variable region can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified Lipin3 polypeptides are made using established procedures in the art and can be screened using standard assays known in the art.

The invention also encompasses fusion proteins comprising one or more Lipin3 polypeptides. For purposes of this invention, a Lipin3 fusion protein contains one or more Lipin3 polypeptides and another amino acid sequence to which it is not attached in the native molecule, for example, a heterologous sequence or a homologous sequence from another region. Useful heterologous sequences include, but are not limited to, sequences that provide for secretion from a host cell, enhance immunological reactivity, or facilitate the coupling of the polypeptide to an immunoassay support or a vaccine carrier. For example, a useful heterologous sequence is one which facilitates purification. Examples of such sequences are known in the art and include those encoding epitopes such as Myc, HA (derived from influenza virus hemagglutinin), His-6, or FLAG. Other heterologous sequences that facilitate purification are derived from proteins such as glutathione S-transferase (GST), maltose-binding protein (MBP), or the Fe portion of immunoglobulin.

The invention also encompasses polymeric forms of Lipin3 polypeptides, preferably full-length Lipin3 polypeptides. As used herein, a polymeric form of a Lipin3 polypeptide contains a plurality (i.e., more than one) of Lipin3 polypeptides. In one embodiment, linear polymers of Lipin3 polypeptides are provided. These Lipin3 linear polymers may be conjugated to carrier. These linear polymers can comprise multiple copies of a single Lipin3 polypeptide, or combinations of different Lipin3 polypeptides, and can have tandem Lipin3 polypeptides, or Lipin3 polypeptides separated by other amino acid sequences. These linear polymers can be made using standard recombinant methods well known in the art.

Lipin3 polypeptides of the invention can be identified and/or characterized in a number of ways. For example, a Lipin3 polypeptide can be tested for its ability to bind to, for instance, another protein (such as an antibody or a protein associated with gene regulation by interacting with full-length Lipin3). Alternatively, Lipin3 polypeptide(s) can be tested for its ability to elicit an immune response, whether humoral or cellular. A Lipin3 polypeptide may also be tested for its ability to elicit one or more characteristics associated with LIPIN3 function, including increased or decreased body mass (as measured, for example, by body mass index, or “BMI”), increased or decreased waist diameter, increased or decreased fat cell density or number, altered anthropometry, basal metabolic rates, or total energy expenditure, chronic disruption of the energy balance, increased Fat Mass as determined, for example, by DEXA (Dexa Fat Mass percent), altered maximum oxygen use (VO2), high fat oxidation, high relative resting rate, glucose resistance, hyperlipidemia, insulin resistance, and hyperglycemia. See also, for example, Hopkinson et al. (1997) Am J Clin Nutr 65(2): 432-8.; Butte et al. (1999) Am J Clin Nutr 69(2): 299-307. It is understood that only one of these properties need be present in order for a polypeptide to come within this invention, although more than one of these properties may be present. Screening such polypeptides is well within the skill of the art.

The ability of a Lipin3 polypeptide to bind (i.e., interact with) another protein can be assessed using standard techniques in the art. Binding of a Lipin3 polypeptide to an antibody may be assessed, for example, by RIA (i.e., by reacting radiolabeled Lipin3 polypeptide with an antibody that is coated on microtiter plates). In another procedure, binding to an antibody is determined by competitive immunoassay. For example, a fragment is tested for its ability to interfere with the binding between the antibody and another polypeptide known to bind to the antibody. This assay may be conducted by labeling one of the components (i.e., antibody or polypeptide known to bind to the antibody), and optionally immobilizing the other member of the binding pair on a solid support for ease of separation. The test fragment is incubated with labeled region, and then the mixture is presented to the immobilized target to determine if the test fragment is able to inhibit binding.

A Lipin3 polypeptide can also be used to identify Lipin3 interacting proteins, which are candidate drug targets for treatment of obesity and obesity-related diseases. In the case of testing whether the Lipin3 polypeptide binds to another protein, for instance, a protein known to be involved in a LIPIN3 pathway, or a protein known to bind to Lipin3, assays to detect binding are known in the art and need not be described in detail herein. For instance, a protein is immobilized on a suitable column. Extracts or solutions containing the test Lipin3 polypeptide are then run through the column, and a determination is made whether the Lipin3 polypeptide was retained on the column. Conversely, the Lipin3 polypeptides can be immobilized on a column and cell extracts or lysates are allowed to run through the column.

Agonists or antagonists of the polypeptides of the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of a polypeptide binding partner, such as a receptor, encoded by a gene or a cDNA corresponding to a polynucleotide described herein, and at least one peptide agonist or antagonist of the binding partner. Such agonists and antagonists can be used to modulate, enhance, or inhibit receptor function in cells to which the receptor is native, or in cells that possess the receptor as a result of genetic engineering. Further, if the receptor shares biologically important characteristics with a known receptor, information about agonist/antagonist binding can facilitate development of improved agonists/antagonists of the known receptor.

Compositions containing Lipin3 polypeptides are encompassed by this invention. When these compositions are to be used pharmaceutically, they are combined with a pharmaceutically acceptable excipient. Examples of pharmaceutically acceptable excipients are known in the art. When these compositions are to be used for detection, they are combined with a suitable substance such as a buffer, and they contain an amount effective to allow detection. Array comprising Lipin3 polypeptides are encompassed by this invention and are described herein.

Preparation of Polypeptides of this Invention

The polypeptides of this invention can be made by procedures known in the art. The polypeptides can be produced by recombinant methods (i.e., single or fusion polypeptides) or by chemical synthesis. Polypeptides, especially shorter polypeptides up to about 50 amino acids, are conveniently made by chemical synthesis. Methods of chemical synthesis are known in the art and are commercially available. For example, a polypeptide could be produced by an automated polypeptide synthesizer employing the solid phase method. Polypeptides can also be made by chemical synthesis using techniques known in the art. Peptide libraries can be synthesized according to methods known in the art (see, e.g., U.S. Pat. No. 5,010,175, and WO 91/17823).

Polypeptides can also be made by expression systems, using recombinant methods. The availability of polynucleotides encoding polypeptides permits the construction of expression vectors encoding intact (i.e., native) polypeptide, functionally equivalent fragments thereof, or recombinant forms. A polynucleotide encoding the desired polypeptide, whether in fused or mature form, and whether or not containing a signal sequence to permit secretion, may be ligated into expression vectors suitable for any convenient host. Both eukaryotic and prokaryotic host systems can be used. The polypeptide is then isolated from lysed cells or from the culture medium and purified to the extent needed for its intended use. Purification or isolation of the polypeptides expressed in host systems can be accomplished by any method known in the art. For example, cDNA encoding a polypeptide intact or a fragment thereof can be operatively linked to a suitable promoter, inserted into an expression vector, and transfected into a suitable host cell. The host cell is then cultured under conditions that allow transcription and translation to occur, and the desired polypeptide is recovered. Other controlling transcription or translation segments, such as signal sequences that direct the polypeptide to a specific cell compartment (i.e., for secretion), can also be used. Examples of prokaryotic host cells are known in the art and include, for example, E. coli and B. subitilis. Examples of eukaryotic host cells are known in the art and include yeast, avian, insect, plant, and animal cells such as COS7, HeLa, CHO and other mammalian cells.

When using an expression system to produce Lipin3 polypeptides, it is often preferable to construct a fusion protein that facilitates purification. Examples of components for these fusion proteins include, but are not limited to myc, HA, FLAG, His-6, glutathione S-transferease, maltose binding protein or the Fc portion of immunoglobulin. These methods are known in the art.

Preferably, especially if used for diagnostic purposes, the polypeptides are at least partially purified or isolated from other cellular constituents. Preferably, the polypeptides are at least 50% pure. In this context, purity is calculated as a weight percent of the total protein content of the preparation. More preferably, the proteins are 50-75% pure. More highly purified polypeptides may also be obtained and are encompassed by the present invention. For clinical use, the polypeptides are preferably highly purified, at least about 80% pure, and free of pyrogens and other contaminants. Methods of protein purification are known in the art and are not described in detail herein.

Uses of Polypeptides

The polypeptides of this invention have a variety of uses. They can be used to identify proteins which bind or interact with Lipin3. They can also be used, for example, to detect the presence of an antibody that binds to these polypeptide(s) or fragment(s) thereof. They may also be used to raise antibodies in a suitable host, which may be rabbit, mouse, rat, goat, or human, as non-inclusive examples. They are also useful as targets for therapeutic intervention. Because over-expression of LIPIN3 has been discovered to be associated with obesity, elevated levels of expression of Lipin 3 polypeptides encoded by LIPIN3 may likely play a role in obesity.

Lipin3 compositions may be used as biochemical markers of that indicate propensity to obesity, the signs of obesity or an obesity related disease, and/or to monitor the efficacy of various therapies and preventative interventions. Diagnostic (i.e., detection) and screening methods are described in more detail below. Lipin3 compositions useful for assessing expression of Lipin3, or a fragment thereof, are also encompassed by the present invention.

Lipin3 polypeptides may also be used an agent to screen pharmaceutical candidates (both in vitro and in vivo), for rational (i.e., structure-based) drug design, as well as possible therapeutic uses as described above. Uses in pharmaceutical development will be described in more detail below. The Lipin3 polypeptides may also be used to identifying proteins, especially those from humans, that bind (or interact physically) with Lipin3 and could thus themselves be drug targets.

Lipin3 polypeptides may also be immobilized on a surface, e.g., an array. Such arrays are useful for screening, e.g. for Lipin3 binding partners.

Arrays Comprising Lipin 3 Polypeptides

The invention also encompasses an array comprising the Lipin3 polypeptides of the invention, as described herein. Therefore, in one aspect, the invention provides an array comprising at least two Lipin3 polypeptides encoded by a polynucleotide of the invention as described herein. Another embodiment comprises an array comprising at least two polypeptides comprising the sequence shown in Table 9, or a region thereof, wherein the region is at least 5 contiguous amino acids in length (or more, e.g. at least 10, 15, 25, 50, 75, 100, 150, or more amino acids in length). In another embodiment, the array further comprises at least one polypeptide selected from one or more of the group consisting of a Lipin1 polypeptide, a Lipin2 polypeptide, a mouse Lipin3 polypeptide, a polypeptide shown in Table 3, a polypeptide shown in Table 4, a polypeptide shown in Table 5, a polypeptide shown in Table 6, or a polypeptide shown in Table 7A, or regions thereof

The invention also encompasses as array comprising an isolated polypeptide comprising the polypeptide sequence shown in Table 10 or a region of at least 5 contiguous amino acids in length (or more, e.g., at least 10, 15, 25, 50, 75, 100, 150, amino acids in length) of the polypeptide sequence shown in Table 10, wherein the region contains amino acid 634.

Antibodies and Their Preparation

Also provided by this invention are antibodies capable of specifically binding to Lipin3 polypeptide(s) of this invention. The antibodies can be useful for, for example, diagnostic purposes, as described more fully below. Antibodies of this invention can also be used for purification and/or isolation of polypeptides described herein.

In one embodiment, the invention provides an antibody capable of specifically binding to a polypeptide of this invention. In another embodiment, the antibody is capable of specifically binding to a polypeptide comprising (a) a polypeptide having the sequence shown in Table 9; or (b) amino acids 588-609 shown in Table 9. The present invention also provides an antibody that is capable of specifically binding to a region of the polypeptide shown in Table 9 or Table 7A, wherein said region is at least about 5 contiguous amino acids in length. For example, the invention includes an antibody that is capable of specifically binding to at least 10, at least 15, at least 20, at least 25, or more contiguous amino acids of a sequence depicted in Table 9 or Table 7A.

In another embodiment, the invention provides an antibody that is capable of specifically binding to a polypeptide comprising a region of at least 5 contiguous amino acids of the polypeptide shown in Table 9, said region comprising amino acids 438 and 439 shown in Table 9. In yet another embodiment, the invention provides an antibody that is capable of specifically binding to a polypeptide comprising a region of at least 5 contiguous amino acids of the peptide shown in Table 9, said region comprising amino acids 587 and 588 shown in Table 9.

In another embodiment, the invention provides an antibody that is capable of specifically binding to a polypeptide comprising a region of at least about 5 contiguous amino acids of the polypeptide sequence shown in Table 9, said region comprising amino acids 21 and 22 shown in Table 9. In another embodiment, the invention provides an antibody that is capable of specifically binding to a polypeptide comprising a region of at least about 5 contiguous amino acids of the polypeptide sequence shown in Table 9, said region comprising amino acids 95 and 96 shown in Table 9. In another embodiment, the invention provides an antibody that is capable of specifically binding to a polypeptide comprising a region of at least about 5 contiguous amino acids of the polypeptide sequence shown in Table 9, said region comprising amino acids 356 and 357 shown in Table 9. For example, the invention includes an antibody that is capable of specifically binding to a polypeptide comprising a at least 10, at least 15, at least 20, at least 25, or more contiguous amino acids of a sequence depicted in Table 9 or Table 7A.

In one embodiment, the invention provides an antibody capable of specifically binding to an altered (mutant) polypeptide of this invention. In one embodiment, the isolated polypeptide comprising a polypeptide having the amino acid sequence shown in Table 10, or a region consisting at least about 5 contiguous amino acids (or more, e.g. about 10, about 15, 25, 50, 75, 100, 150, amino acids in length) of the amino acid sequence shown in Table 10, wherein said region includes amino acid 634.

As noted in the definition of “antibody” above, this includes fragments of antibodies, such as Fab fragments. In another embodiment, a monoclonal antibody is provided that is capable of specifically binding to a polypeptide of this invention. In one embodiment, the invention provides a monoclonal antibody capable of specifically binding to a polypeptide of this invention. In another embodiment, the antibody is capable of specifically binding to a polypeptide comprising (a) a polypeptide having the sequence shown in Table 9; or (b) amino acids 588-609 shown in Table 9. The present invention also provides a monoclonal antibody that is capable of specifically binding to a region of the polypeptide shown in Table 9 or Table 7A, wherein said region is at least about 5 contiguous amino acids in length. For example, the invention includes a monoclonal antibody that is capable of specifically binding to at least 10, at least 15, at least 20, at least 25, or more contiguous amino acids of a sequence depicted in Table 9 or Table 7A.

In another embodiment, the invention provides a monoclonal antibody that is capable of specifically binding to a polypeptide comprising a region of at least 5 contiguous amino acids of the polypeptide shown in Table 9, said region comprising amino acids 438 and 439 shown in Table 9. In yet another embodiment, the invention provides a monoclonal antibody that is capable of specifically binding to a polypeptide comprising a region of at least 5 contiguous amino acids of the peptide shown in Table 9, said region comprising amino acids 587 and 588 shown in Table 9.

In another embodiment, the invention provides a monoclonal antibody that is capable of specifically binding to a polypeptide comprising a region of at least about 5 contiguous amino acids of the polypeptide sequence shown in Table 9, said region comprising amino acids 21 and 22 shown in Table 9. In another embodiment, the invention provides a monoclonal antibody that is capable of specifically binding to a polypeptide comprising a region of at least about 5 contiguous amino acids of the polypeptide sequence shown in Table 9, said region comprising amino acids 95 and 96 shown in Table 9. In another embodiment, the invention provides a monoclonal antibody that is capable of specifically binding to a polypeptide comprising a region of at least about 5 contiguous amino acids of the polypeptide sequence shown in Table 9, said region comprising amino acids 356 and 357 shown in Table 9. For example, the invention includes a monoclonal antibody that is capable of specifically binding to a polypeptide comprising a at least 10, at least 15, at least 20, at least 25, or more contiguous amino acids of a sequence depicted in Table 9 or Table 7A. Laboratory methods for producing polyclonal antibodies and monoclonal antibodies, as well as deducing their corresponding nucleic acid sequences, are known in the art. For example, see Harlow and Lane, Antibodies: A Laboratory Manual, Cold Spring Harbor Laboratory, New York (1988) and Sambrook et al. (1989).

The antibodies of this invention may be polyclonal or monoclonal. Monoclonal antibodies of this invention can be biologically produced by introducing a polypeptide (or fragment of a polypeptide) of this invention into an animal, e.g., mouse or rat. The antibody producing cells in the animal are isolated and fused with myeloma cells or heteromyeloma cells to produce hybrid cells or hybridomas. Accordingly, the invention also includes hybridoma cells producing the monoclonal antibodies of this invention.

Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. 82:8653 or Spira et al. (1984) J. Immunol. Methods 74:307.

Thus, using the polypeptide(s) of this invention or fragment(s) thereof, and well known methods, one of skill in the art can produce and screen the hybridoma cells and antibodies of this invention for antibodies having the ability to bind polypeptide(s) of this invention.

If a monoclonal antibody being tested binds with a Lipin3 polypeptide(s) of this invention, then the antibody being tested and the antibodies provided by the hybridomas of this invention are equivalent. It is also possible to determine without undue experimentation whether an antibody has the same specificity as a monoclonal antibody of this invention by determining whether the antibody being tested prevents a monoclonal antibody of this invention from binding the polypeptide(s) with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with the polypeptide(s) with which it is normally reactive, and determine if the monoclonal antibody being tested is inhibited in its ability to bind the antigen. If the monoclonal antibody being tested is inhibited, then, in all likelihood, it has the same, or a closely related, epitopic specificity as the monoclonal antibody of this invention.

As noted above, this invention also provides biological active fragments of the polyclonal and monoclonal antibodies described above. These antibody fragments retain some ability to selectively bind with its antigen or immunogen. Examples of antibody fragments are known in the art and include, but are not limited to, CDR regions, Fab, Fab′, F(ab′)₂, Fv, and single chain methods. Methods of making these fragments are known in the art, see for example, Harlow and Lane, (1988).

The antibodies of this invention also can be modified to create chimeric antibodies and humanized antibodies (Oi et al. (1986) BioTechniques 4(3):214); U.S. Pat. No. 4,816,567. Chimeric antibodies are those in which the various domains of the antibodies' heavy and light chains are coded for by DNA from more than one species.

The isolation of other hybridomas secreting monoclonal antibodies with the specificity of the monoclonal antibodies of the invention can also be accomplished by one skilled in the art by producing anti-idiotypic antibodies (Herlyn, et al. (1986) Science, 232:100). An anti-idiotypic antibody is an antibody which recognizes unique determinants present on the monoclonal antibody produced by the hybridoma of interest. These determinants are located in the hypervariable region of the antibody. It is this region which binds to a given epitope and, thus, it is responsible for the specificity of the antibody. The anti-idiotypic antibody can be prepared by immunizing an animal with the monoclonal antibody of interest. The animal immunized will recognize and respond to the idiotypic determinants of the immunizing antibody by producing an antibody to these idiotypic determinants. By using the anti-idiotypic antibodies of the second animal, which are specific for the monoclonal antibodies produced by a single hybridoma which was used to immunize the second animal, it is now possible to identify other clones with similar idiotypes as the antibody of the hybridoma used for immunization.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

The antibodies of this invention can be linked (i.e., conjugated) to a detectable agent or a hapten. The complex is useful to detect the polypeptide(s) (or polypeptide fragments) to which the antibody specifically binds in a sample, using standard immunochemical techniques such as immunohistochemistry as described by Harlow and Lane (1988), supra. Examples of types of immunoassays which can utilize monoclonal antibodies of the invention are competitive and non-competitive immunoassays in either a direct or indirect format. Examples of such immunoassays are the enzyme linked immunoassay (ELISA) radioimmunoassay (RIA) and the sandwich (immunometric) assay. Detection of using the monoclonal antibodies of the invention can be done by utilizing immunoassays which are run in either the forward, reverse, or simultaneous modes, including immunohistochemical assays on physiological samples. Those of skill in the art will know, or can readily discern, other immunoassay formats without undue experimentation.

Another technique which may also result in greater sensitivity consists of coupling the antibodies to low molecular weight haptens. These haptens can then be specifically detected by means of a second reaction. For example, it is common to use such haptens as biotin, which reacts avidin, or dinitropherryl, pyridoxal, and fluorescein, which can react with specific anti-hapten antibodies. See Harlow and Lane (1988) supra.

The monoclonal antibodies of the invention can be bound to many different carriers. Thus, this invention also provides compositions containing antibodies and a carrier. Carriers can be active and/or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, and bioluminescent compounds. Those of ordinary skill in the art will know of other suitable labels for binding to the monoclonal antibody, or will be able to ascertain such, using routine experimentation. Furthermore, the binding of these labels to the monoclonal antibody of the invention can be done using standard techniques common to those of ordinary skill in the art.

For purposes of the invention, polypeptides of this invention may be detected by the monoclonal antibodies of the invention when present in biological samples, such as fluids and tissues. This use of antibodies is discussed in more detail below.

Compositions containing the antibodies, fragments thereof or cell lines which produce the antibodies, are encompassed by this invention. When these compositions are to be used pharmaceutically, they are combined with a pharmaceutically acceptable excipient. Arrays comprising the antibodies or fragments thereof are encompassed by this invention. Antibodies may be immobilized on a surface, e.g., an array for use in detection and diagnostic assays as described in more detail below.

Cells with Altered LIPIN3 Expression

Cells in which LIPIN3 expression is increased are useful for screening for agents that decrease LIPIN3 expression (e.g., reduce LIPIN3 overexpression). Because LIPIN3 overexpression has been associated with obesity, agents that decrease LIPIN3 overexpression are candidate antiobesity drugs. Similarly, cells in which LIPIN3 expression is inhibited or prevented are useful for identifying agents that mimic LIPIN3 activity and/or act in a LIPIN3 pathway.

Cells comprising LIPIN3 polynucleotides are described supra.

In addition, the present invention comprises substantially purified cells in which LIPIN3 expression is increased or decreased (or prevented or eliminated). As is understood in the art, a relative increase or decrease in LIPIN3 expression can be determined by comparison to suitable control cells. Another embodiment provides substantially purified cells in which LIPIN3 expression is inhibited or prevented. The cells are preferably of mammalian, primate, mouse, or human origin, but cells derived from other species may also be employed. The cells can comprise cells capable of stable growth through several passages in culture and/or cells grown for fewer or no passages in culture. The cells can be capable of sustained or indefinite growth in culture (for example, stable cell lines) or not capable of stable growth in culture (e.g., tissue explants).

The present invention also encompasses cells containing recombinant DNA sequence which comprises a LIPIN3 polynucleotide or a mutant LIPIN3 polynucleotide. In one embodiment, the invention is a genetically engineered mammalian cell comprising a recombinantly modified LIPIN3 polynucleotide, such that the polynucleotide is overexpressed. In another embodiment, a LIPIN3 polynucleotide is operatively linked to an inducible promoter. In still other embodiments, the genetically engineered cells possess a mutant LIPIN3 gene instead of a native LIPIN3 gene. The resulting cells are termed “knock-in” if the resulting cell is left with an altered LIPIN3 polynucleotide instead of a native LIPIN3 gene. Examples of mutant LIPIN3 polynucleotides are described herein, and include without limitation, a LIPIN3 polynucleotide as shown in Table 8.

In another embodiment, the genetically engineered cells possess a native (wildtype) LIPIN3 polynucleotide sequence that is disrupted such that expression of a native Lipin3 gene product is inhibited or prevented. For example, the native LIPIN3 DNA sequence may be removed or replaced as a result of interaction with an recombinant DNA sequence. Such cells include LIPIN3 “knock-out” cells, if the resulting cell is left without a native DNA that encodes a functional LIPIN3 gene product.

The cells are preferably of mammalian, primate, mouse, or human origin, but cells derived from other species may also be employed. The cells can comprise cells capable of stable growth through several passages in culture and/or cells grown for fewer or no passages in culture. The cells can be capable of sustained or indefinite growth in culture (for example, stable cell lines) or not capable of stable growth in culture (e.g., tissue explants). Any cell or cell line, the genotype of which has been altered by the presence of a recombinant DNA sequence is encompassed by the invention. Devising cells that lack LIPIN3 (or its homolog) function or overexpress LIPIN3 polynucleotides or comprise mutant LIPIN3 polynucleotides may be achieved in a variety of ways, including, but not limited to, genetic manipulation such as deletion mutagenesis, recombinant substitution of a functional portion of the gene, frameshift mutations, conventional or classical genetic techniques pertaining to mutant isolation, or alterations of the regulatory domains. General methods for constructing recombinant DNA which can transform target cells are also well known in the art, and further discussed herein. Methods for producing “knock-out” and “knock-in” cells are also well known in the art.

Methods Using LIPIN3 Polynucleotides, Polypeptides, and Antibodies: Detection Systems

The invention also provides methods using the LIPIN3 polynucleotides, polypeptides, and/or antibodies of the invention to detect suitable targets in a biological sample. Procedures for conducting diagnostic (i.e., detection) tests using polynucleotides, polypeptides or antibodies are extensively known in the art and are routine for a practitioner or ordinary skill. Generally, to perform a diagnostic method of this invention, one of the compositions of this invention is provided as a reagent to detect a target with which it reacts in a biological sample. The target is supplied by obtaining a suitable biological sample from an individual for whom the diagnostic parameter is to be measured. Many types of samples are suitable for this purpose. If desired, the target may be partially purified from the sample or amplified before the assay is conducted.

Polynucleotides

LIPIN3 polynucleotides can also be used as hybridization probes for detection of the presence of and/or differential expression of LIPIN3 polynucleotides. For example, increased expression of LIPIN3 polynucleotides has been associated with obesity. Therefore, in one embodiment, the invention provides methods for detecting altered expression of a LIPIN3 polynucleotide in a test sample comprising detecting a level of expression of any of the LIPIN3 polynucleotide as described herein. In some embodiments, the altered expression is increased expression. In another embodiment, the invention provides methods for detecting altered expression of a LIPIN3 polynucleotide in a test sample comprising detecting a level of expression of any of the LIPIN3 polynucleotide as described herein; and determining whether the expression of the LIPIN3 polynucleotide is altered in the test sample compared to expression of a LIPIN3 polynucleotide in a normal sample. In another embodiment, the invention provides a method for detecting differential expression of a LIPIN3 polynucleotide in a test sample comprising: detecting a level of expression of a LIPIN3 polynucleotide as described herein; comparing the level of expression of the polynucleotide in the test sample with the level of expression of the polynucleotide in a control cell sample; and determining the presence of differential expression of the polynucleotide in the test cell sample relative to the polynucleotide in the control cell sample, if any. Another embodiment of the invention provides a method of detecting differential expression of a LIPIN3 polynucleotide in a test sample comprising: detecting a level of expression of (i) a polynucleotide comprising the sequence shown in Table 1; (ii) a polynucleotide comprising the sequence shown in Table 7B; or (iii) a region of a polynucleotide comprising the sequence shown in Table 1 or Table 7B, wherein said region is at least about 10 nucleotides in length (or more, e.g., about 15, 18, 20, 25, 30, 35, 50, 100 or more nucleotides in length); comparing the level of expression of the polynucleotide in the test sample with the level of expression of the polynucleotide in a control cell sample; and determining the presence of differential expression of the polynucleotide in the test cell sample relative to the polynucleotide in the control cell sample, if any. In another embodiment, the differential expression is LIPIN3 overexpression.

LIPIN3 polynucleotide can also be used as probes for the detection of the presence of a mutant LIPIN3 gene. As shown in Example 5, a mutant LIPIN3 sequence has been associated with obesity in a population of obese women. Therefore, the invention provides a method of detecting an altered LIPIN3 polynucleotide comprising: detecting expression of a polynucleotide comprising: (i) a comprising the sequence shown in Table 8, or (ii) a region of a polynucleotide comprising the sequence shown in Table 8, wherein said region comprises nucleotide 1904. In another embodiment, the invention provides a method of detecting a mutation in the LIPIN3 gene, comprising screening for a specific mutation (alteration) in the LIPIN3 gene or determining a level of expression of the LIPIN3 gene is a tissue sample from a subject, whereby an altered level of expression indicates that a mutant LIPIN3 gene is present.

FIG. 1 and Table 13 describe four other polymorphisms identified in a population of obese women: the G at position 18,964 is changed to A; the C at position 19, 103 changed to T, the T at position 28,634 is changed to C; and the C at position 28,726 is changed to T. Thus, in other embodiments, the invention provides a method of detecting an altered LIPIN3 polynucleotide comprising: detecting expression of a polynucleotide comprising (i) a region of a polynucleotide comprising the sequence shown in FIG. 1, wherein said region comprises an A nucleotide at position 18, 964. In another embodiment, the invention provides a method of detecting an altered LIPIN3 polynucleotide comprising: detecting expression of a polynucleotide comprising (i) a region of a polynucleotide comprising the sequence shown in FIG. 1, wherein said region comprises a T nucleotide at position 19, 103. In another embodiment, the invention provides a method of detecting an altered LIPIN3 polynucleotide comprising: detecting expression of a polynucleotide comprising (i) a region of a polynucleotide comprising the sequence shown in FIG. 1, wherein said region comprises a C nucleotide at position 28,726. In another embodiment, the invention provides a method of detecting an altered LIPIN3 polynucleotide comprising: detecting expression of a polynucleotide comprising (i) a region of a polynucleotide comprising the sequence shown in FIG. 1, wherein said region comprises a T nucleotide at position 28,872.

For these methods, a suitable cell sample or a sample derived from cells (either of which are suspected of containing LIPIN3 polynucleotide sequences) is obtained and tested for the presence of LIPIN3 polynucleotide by contacting the polynucleotides from the sample with the LIPIN3 polynucleotide probe. The method is conducted to allow hybridization to occur between the LIPIN3 probe and LIPIN3 polynucleotide of interest, and the resultant (if any) hybridized complex is detected. Such methods entail procedures well known in the art, such as cell culture, polynucleotide preparation, hybridization, and detection of hybrid complexes formed, if any. Using similar methods, the probes can also be used to detect vectors which are in turn used to produce LIPIN3 polypeptides, intact LIPIN3, or recombinant, variant forms of LIPIN3.

The reaction is performed by contacting a LIPIN3 polynucleotide under conditions that will allow a stable complex to form between the LIPIN3 polynucleotide and a polynucleotide target. Complex formation is detected by a number of techniques known in the art.

The assay result is preferably compared with a control, e.g., a similar assay conducted on a control sample, preferably a sample from a healthy individual, i.e., an individual that is not obese and/or does not have an obesity related disorder (negative control). The assay can be conducted on the test sample and control sample simultaneously. Presence of differential LIPIN3 expression is of particular interest because increased LIPIN3 expression has been correlated with obesity.

These diagnostic assays may be rendered specific by, for example (a) performing a hybridization reaction with a specific probe; (b) performing an amplification with a specific primer; or (c) combination of (a) and (b). To perform an assay that is specific due to hybridization with a specific probe, a polynucleotide is chosen with the required degree of complementarity for the intended target polynucleotide. Preferred probes include polynucleotides of at least about 10 nucleotides in length. These probes may contain the coding or non-coding sequence of LIPIN3. Increasingly preferred are probes comprising at least about 15, 18, 20, 25, 30, 50 or 100 or more polynucleotides.

The probe or primer may be provided with a label. Some of the labels often used include radioisotopes such as ³²P and ³³P, chemiluminscent or fluorescent reagents such as fluorescein, and enzymes such as alkaline phosphatase that are capable of producing a colored solute or precipitant. The label may be intrinsic to the reagent, it may be attached by direct chemical linkage, or it may be connected through a series of intermediate reactive molecules, such as a biotin-avidin complex, or a series of inter-reactive polynucleotides. The label may be added to the reagent before hybridization with the target polynucleotide, or afterwards. To improve the sensitivity of the assay, it is often desirable to increase the signal ensuing from hybridization. This can be accomplished by using a combination of serially hybridizing polynucleotides or branched polynucleotides in such a way that multiple label components become incorporated into each complex. See U.S. Pat. No. 5,124,426.

If desired, the target polynucleotide may be extracted from the sample, and may also be partially purified. The target polynucleotide may be optionally subjected to any combination of additional treatments, including digestion with restriction endonucleases, size separation (by electrophoresis in agarose or polyacrylamide, for example), and affixation to a reaction matrix, such as a blotting material.

Hybridization is allowed to occur by mixing the LIPIN3 polynucleotide with a sample suspected of containing target polynucleotide under appropriate reaction conditions. This may be followed by washing or separation to remove unreacted reagent. Generally, both target polynucleotide and LIPIN3 polynucleotide are at least partly equilibrated into the single-stranded form (i.e., denatured) in order for complementary sequences to hybridize efficiently.

The level of hybridization stringency depends, inter alia, upon the objective of the test and the particular LIPIN3 polynucleotide used. For example, a preferred set of conditions for used with a probe of 50 nucleotides or more is 6×SSC at 37° C. in 50% formamide, followed by a wash at low ionic strength. This will generally require that the polynucleotide target be at least about 90% identical with the LIPIN3 polynucleotide for a stable duplex to form. The specificity of the reaction may also be increased by increasing the length of the LIPIN3 polynucleotide used.

Appropriate hybridization conditions are determined to permit hybridization of the desired specificity. Conditions may be estimated beforehand using the formula given above. Preferably, LIPIN3 probes share little to no sequence homology with LIPIN1 or LIPIN2 sequences. However, even if there are shared sequences, such a probe may still be useful if detection systems allow discrimination between signal due to hybridization to LIPIN3 sequences and signal due to hybridization to other sequence, including LIPIN1 and/or LIPIN2 sequences. If it is additionally desirable to distinguish between and/or among various species, for example between the mouse and human LIPIN3 sequences, the probe (due to length and/or sequence content) and/or hybridization conditions should be adjusted and selected such that these sequences may be distinguished. It is also occasionally desirable to use probes capable of detecting LIPIN1, LIPIN2 and LIPIN3.

Another method of detecting polynucleotide target is by using PCR. All processes of producing replicate copies of the same polynucleotide, such as PCR or gene cloning, are collectively referred to herein as “replication”. PCR primers consisting of sequences unique to LIPIN3 may be used to amplify any such sequences in the sample. Preferably, a sample known not to contain any LIPIN3 sequences, or a control from a normal individual, is used as a negative control. PCR methods are well known in the art and need not be described herein. For these methods, DNA or RNA is prepared from a sample. Quantitative methods of PCR are well known in the art, and can be used to detect differential expression, e.g. overexpression, of a LIPIN3 polynucleotide in a test sample relative to expression of a LIPIN3 polynucleotide in a control sample. Other amplification methods may be used for detection and are known in the art.

The primers used can consist of regions of the polynucleotide shown in Table 1, Table 7B or FIG. 1. A non-limiting list of exemplary primers is shown in Table 12. Preferably, at least one, preferably both, of the primers are sequences unique to LIPIN3. Alternatively, if the expected size of the amplified reaction product is known and different from that of the non-target (for example, LIPIN1) polynucleotides, the sequences of the primers need not be unique. Generally, the primer is about 15 to 20 nucleotides in length, although longer primer of 30 to 50 (or even longer) nucleotides may be used. In other embodiments, the invention provides a primer with at least 5, 10, 15, 20, 25, 25, or 30 or more contiguous nucleotides of any LIPIN3 polynucleotide described herein. In some embodiments, the primer sequence is sufficiently near nucleotide 1304 of Table 8, such that extension of the primer would replicate (copy) the polymorphism(s). In other embodiments, the primer sequence is sufficiently near nucleotide 18,964 of FIG. 1, nucleotide 19,103 of FIG. 1, nucleotide 28,634 of FIG. 1, and/or nucleotide 28,872 of FIG. 1, such that extension of the primer would replicate (copy) the polymorphism(s). Kits comprising one or more of these primers could be useful for detection of these polymorphisms.

A positive test may be indicated by the presence of sufficient reaction product at the end of the amplification series. Amplified polynucleotide may be detected on an agarose gel upon staining with ethidium bromide. Alternatively, a radiolabeled substrate may be added to the mixture during the final amplification cycle. The incorporated label may be separated from unincorporated label (e.g., by blotting or by size separation) and the label may be detected by, for example, counting or autoradiography. If run on a gel of agarose or polyacrylamide, the size of the product may help confirm the identity of the amplified region. Specific amplification may also be followed by specific hybridization, by using the amplification mixture obtained from the foregoing procedure as a target source for the hybridization reaction outlined above.

Additionally, it is possible to assay expression of a LIPIN3 polynucleotide “in situ”, i.e., directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, “PCR In Situ Hybridization: Protocols And Applications”, Raven Press, New York).

Polynucleotide arrays provide a high throughput technique that can assay a large number of polynucleotides or polypeptides in a sample. This technology can be used as a tool to test for, for example, differential expression. A variety of methods of producing arrays, as well as variations of these methods, are known in the art and contemplated for use in the invention. Methods of detection of polynucleotides using polynucleotides immobilized on a surface, e.g. an array, are further described above. Polynucleotides may also be detected using method well known in the art, including TMA, bDNA, NAT or Nasbau.

Antibodies

An antibody embodied in this invention can also be used as a reagent in diagnosis and/or clinical management to detect expression levels and/or alteration in expression of Lipin3 polypeptides. Any such polypeptide can be detected in clinical samples by immunochemical and/or immunohistological techniques that will be apparent to a practitioner of ordinary skill.

Accordingly, the invention includes methods for detecting a Lipin3 polypeptide (i.e., a polypeptide of this invention) in a biological sample, in which the steps are: (a) contacting polypeptide from the sample with an anti-Lipin3 antibody described herein under conditions that permit the formation of a stable antigen-antibody complex and (b) detecting stable complexes formed, if any. The present invention provides methods for detecting the presence of and/or measuring a level of polypeptide in a biological sample, which polypeptide is encoded by a polypeptide that is differentially expressed in a cell from an individual with obesity or an obesity-related disease. Any of a variety of known methods can be used for detection, including, but not limited to, immunoassay, using antibody that binds the polypeptide, e.g. by enzyme-linked immunosorbent assay (ELISA), radioimmunoassay (RIA) and the like; and functional assay for the encoded polypeptide, e.g. binding activity or enzymatic assay.

The antibody used as a reagent may be provided directly with a suitable label. More frequently, the antibody is detected using one of a number of developing reagents which are easily produced or available commercially. Typically, these developing reagents are anti-immunoglobulin or protein A, and they typically bear labels which include, but are not limited to, fluorescent markers such as fluorescein, enzymes such as peroxidase that are capable of precipitating a suitable chemical compound, or that emits light by way of a chemical reaction, electron dense markers such as colloidal gold, or radioisotopes such as ¹²⁵I, ³²P, or ³⁵S.

The amount of polypeptide may be detected in a standard quantitative immunoassay. If the protein is secreted or shed from the cell in any appreciable amount, or is present in white blood cells, it may be detectable in plasma or serum samples. Alternatively, the target protein may be solubilized or extracted from a solid tissue sample. Before quantitating, the protein may optionally be affixed to a solid phase, such as by a blot technique or using a capture antibody.

A number of immunoassay methods are established in the art for performing the quantitation. For example, the protein may be mixed with a pre-determined non-limiting amount of the reagent antibody specific for the protein. The reagent antibody may contain a directly attached label, such as an enzyme or a radioisotope, or a second labeled reagent may be added, such as anti-immunoglobulin or protein A. For a solid-phase assay, unreacted reagents are removed by washing. For a liquid-phase assay, unreacted reagents are removed by some other separation technique, such as filtration or chromatography. The amount of label captured in the complex is positively related to the amount of target protein present in the test sample. A variation of this technique is a competitive assay, in which the target protein competes with a labeled analog for binding sites on the specific antibody. In this case, the amount of label captured is negatively related to the amount of target protein present in a test sample. Results obtained using any such assay on a sample from a suspected infected source are compared with those from a non-infected source.

Methods of Detecting Polypeptides of the Invention

The present invention provides methods for detecting the presence of and/or measuring a level of polypeptide in a biological sample, which polypeptide is encoded by a Lipin3 polynucleotide that is differentially expressed in a cell from an individual with obesity or an obesity-related disease. Thus, in one embodiment, the invention provides methods for detecting expression of a Lipin3 polypeptide in a test sample comprising detecting a level of expression of any of the LIPIN3 polypeptides as described herein. In some embodiments, the expression is increased expression. In another embodiment, the invention provides methods for detecting expression of a Lipin3 polypeptide in a test sample comprising detecting a level of expression of any of the Lipin3 polypeptides as described herein; and determining whether the expression of the Lipin3 polypeptide is altered in the test sample compared to expression of a Lipin3 polypeptide in a normal sample. In one embodiment, the invention provides a method for detecting differential expression of a Lipin3 polypeptide in a test sample comprising: detecting a level of expression of at least one polypeptide encoded by a LIPIN3 polynucleotide as described herein; comparing the level of expression of the polypeptide in the test sample with the level of expression of the polypeptide in a control sample; and determining the presence of differential expression of the polypeptide in the test cell sample relative to the polypeptide in the control sample, if any. In another embodiment, the differential expression is overexpression. Any of a variety of known methods can be used for detection, including, but not limited to, mass spectroscopy, capillary electrophoresis, and the antibody-based methods as described in detail above.

Methods of Diagnosis, Aiding Diagnosis, and Monitoring Obesity and Obesity-Related Diseases

The invention also provides methods for detecting obesity, or an obesity-related disease, associated with altered (preferably, elevated) levels of LIPIN3 polynucleotides, by means of detecting polynucleotides of the invention. In one embodiment, the invention provides a method of diagnosing obesity associated with differential expression of a LIPIN3 polypeptide. Therefore, in one embodiment, the invention provides methods for detecting differential expression of a LIPIN3 polynucleotide in a test sample comprising detecting a level of expression of any of the LIPIN3 polynucleotide as described herein. In some embodiments, the expression is increased expression. In another embodiment, the invention provides methods for detecting differential expression of a LIPIN3 polynucleotide in a test sample comprising detecting a level of expression of any of the LIPIN3 polynucleotide as described herein; and determining whether the expression of the LIPIN3 polynucleotide is altered in the test sample compared to expression of a LIPIN3 polynucleotide in a normal sample. In another embodiment, the invention provides a method for detecting differential expression of a LIPIN3 polynucleotide in a test sample comprising: detecting a level of expression of a LIPIN3 polynucleotide as described herein; comparing the level of expression of the polynucleotide in the test sample with the level of expression of the polynucleotide in a control cell sample; and determining the presence of differential expression of the polynucleotide in the test cell sample relative to the polynucleotide in the control cell sample, if any. Another embodiment of the invention provides a method of detecting differential expression of a LIPIN3 polynucleotide in a test sample comprising: (i) a polynucleotide comprising the sequence shown in Table 1; (ii) a polynucleotide comprising the sequence shown in Table 7B; or a region of a polynucleotide comprising the sequence shown in Table 1 or in Table 7B, wherein said region is at least about 10 nucleotides in length (or more, e.g., about 15, 18, 20, 25, 30, 35, 50, 100 or more nucleotides in length); comparing the level of expression of the polynucleotide in the test sample with the level of expression of the polynucleotide in a control cell sample; and determining the presence of differential expression of the polynucleotide in the test cell sample relative to the polynucleotide in the control cell sample, if any. In another embodiment, the differential expression is LIPIN3 overexpression.

The invention also provides a method of diagnosing an obesity-related disease by detecting polynucleotides or polypeptides according to the invention, as described herein. The treatment regimen of obesity or a disease associated with obesity may also be monitored by detecting levels of the polynucleotide and polypeptides in order to assess the progression of the disease. The present invention also provides methods of using the polynucleotides described herein for detecting a propensity for obesity or obesity related diseases, facilitating or aiding a diagnosis of obesity and/or obesity diseases and the severity of obesity and/or obesity related diseases, and facilitating or aiding a determination of the prognosis of a subject (e.g., by providing a measure of the therapeutic effect). Detection can be based on detection of a polynucleotide that is differentially expressed in a cell from an individual with obesity or an obesity related disease and/or detection of a polypeptide encoded by a polynucleotide that is differentially expressed in cells from an individual with obesity.

In another aspect, the invention provides methods for aiding risk assessment of obesity or an obesity-related disease by detecting polynucleotides or polypeptides according to the invention, as described herein, by detecting polynucleotides or polypeptides according to the invention, as described herein.

Detection of Mutant LIPIN3 Polynucleotides or Polypeptides

LIPIN3 polynucleotides can also be used as probes for the detection of the presence of a mutant (altered) LIPIN3 gene. A mutant LIPIN3 polynucleotide sequence has been associated with obesity in a population of obese women (as shown in Example 5). Thus, mutant LIPIN3 polynucleotide may be useful for diagnosis or aiding diagnosis of obesity, aiding risk assessment of obesity, propensity to obesity, and prognosis of, for example, severity of obesity and obesity-related diseases, likelihood of complications and the like. While the following discussion is focused on LIPIN3 polynucleotides, it is to be understood that this specificity is only for the purpose of simplicity and clarity. It is contemplated that the methods discussed below are applicable towards mutant LIPIN3 polynucleotides and mutant Lipin3 polypeptides.

Therefore, the invention provides a method of detecting a mutant LIPIN3 polynucleotide comprising: detecting expression of a mutant LIPIN3 polynucleotide. Another embodiment provides a method of detecting a mutant LIPIN3 polynucleotide comprising: detecting expression of a LIPIN3 polynucleotide comprising the sequence shown in Table 8, or a region of a polynucleotide comprising the sequence shown in Table 8, wherein said region comprises nucleotide 1904. In another embodiment, the invention provides a method of detecting a mutation in the LIPIN3 gene, comprising screening for a specific mutation in the LIPIN3 gene or determining a level of expression of the LIPIN3 gene is a tissue sample from a subject, whereby an altered level of expression can indicate that a mutant LIPIN3 gene is present. In another embodiment, the invention provides methods of screening of mutant LIPIN3 polynucleotide.

Methods for detecting mutant polynucleotide sequences are well known in the art, and include, e.g., single strand conformational polymorphism (SSCP), and various sequence amplification based methods for detecting sequence mutations including point mutations, e.g., LCR, NASBA, PCR, limited primer extension, etc. Methods for detecting altered protein sequences include Western blot analysis, capillary electrophoresis, mass spectroscopy, and WAVE. Generally, a detection experiment will be performed in parallel with a control LIPIN3 polynucleotide or polypeptide, or, in the case when altered expression levels are being assessed, with a control sample possessing normal levels of a LIPIN3 polynucleotide or polypeptide.

Methods of Identifying LIPIN3 Mutants

Mutations and polymorphisms in the LIPIN3 gene may be identified using a number of techniques. Nucleic acid from any nucleated cell can be used as the starting point for such assay techniques and may be isolated using standard procedures. Genomic DNA may be used in hybridization or amplification assays of biological samples to detect abnormalities, or alterations, in LIPIN3 gene structure, including point mutations, insertions, deletion and chromosomal rearrangements. Mutations may be found in an exon or intron portion of the gene, or at the exon-intron boundary, or in regulatory sequences including the 5′ and 3′ untranslated regions of the LIPIN3 transcript, as well as in promoter and/or enhancer sequences. Assay for detecting alterations or abnormalities include, but are not limited to, Southern analysis, SSCP analysis, PCR analysis, and the use of a microarray of LIPIN3 nucleic acid sequences immobilized on a chip (see, e.g. Cronin, et al., 1996, Human Mutation 7:244-255). A mutant (non-wildtype) LIPIN3 gene may also be identified by screening for an altered level of expression of a LIPIN3 polynucleotide as described herein. Altered expression of a LIPIN3 polynucleotide may be associated with a mutant LIPIN3 gene, which can be determined using the methods described herein. Thus, the invention provides a method of identifying an alteration in the LIPIN3 gene associated with obesity (or an obesity-related disease) comprising: comparing the sequence of the LIPIN3 gene or level of expression of the LIPIN3 gene in a tissue sample from a subject with the sequence of the wild-type LIPIN3 gene or level of expression of the wild-type LIPIN3 gene, wherein an alteration in the germline sequence or level of expression of the LIPIN3 gene of said subject is associated with obesity. In another embodiment, the detection of an alteration comprises (a) screening for a specific mutation in the LIPIN3 gene in said sample; or (b) determining the level of expression of the LIPIN3 polynucleotide in said sample.

Alternative diagnostic methods for the detection of LIPIN3 gene specific nucleic acid molecules, in patient samples or other appropriate cell sources, may involve their amplification, e.g., by PCR (the experimental embodiment set forth in Mullis, 1987, U.S. Pat. No. 4,683,202), followed by the analysis of the amplified molecules using techniques well known to those of skill in the art, such as, for example, those listed above. The resulting amplified sequences can be compared to those that would be expected if the nucleic acid being amplified contained only normal copies of the LIPIN3 gene in order to determine whether a LIPIN3 gene mutation exists.

Among those LIPIN3 nucleic acid sequences which are preferred for such amplification-related diagnostic screening analyses are oligonucleotide primers which amplify LIPIN3 exon sequences. The sequences of such oligonucleotide primers are, therefore, preferably derived from LIPIN3 intron sequences so that the entire exon, or coding region, can be analyzed as discussed below. Primer pairs useful for amplification of LIPIN3 exons are preferably derived from adjacent introns. Appropriate primer pairs can be chosen such that each of the LIPIN3 exons are amplified. Primers for the amplification of LIPIN3 exons can be routinely designed by one of ordinary skill in the art by utilizing the exon and intron sequences of LIPIN3 shown in FIG. 1. Exemplary primers for amplification of LIPIN3 exons are also shown in Table 12 and FIG. 1.

Additional LIPIN3 nucleic acid sequences which are preferred for such amplification-related analyses are those which will detect the presence of a LIPIN3 polymorphism which differs from the consensus LIPIN3 sequence depicted in Tables, particularly those that detect the polymorphism identified in exon 12 shown in FIG. 1. Such polymorphisms include ones which represent mutations associated with body weight disorders such as obesity, cachexia, or anorexia.

Further, well-known genotyping techniques can be performed to type polymorphisms that are in close proximity to mutations in the LIPIN3 sequence itself, including mutations associated with weight disorders such as obesity, cachexia, or anorexia. Such polymorphisms can be used to identify individuals in families likely to carry mutations in the LIPIN3 gene. If a polymorphism exhibits linkage disequilibrium with mutations in the LIPIN3 gene, the polymorphism can also be used to identify individuals in the general population who are likely to carry such mutations. Polymorphisms that can be used in this way include restriction fragment length polymorphisms (RFLPs), which involve sequence variations in restriction enzyme target sequences, single-base polymorphisms, and simple sequence length polymorphisms (SSLPs).

For example, Weber (U.S. Pat. No. 5,075,217) describes a DNA marker based on length polymorphisms in blocks of (dC-A)ₙ-(dG-dT)ₙ short tandem repeats. The average separation of (dC-dA)ₙ-(dG-dT)ₙ blocks is estimated to be 30,000-60,000 bp. Markers that are so closely spaced exhibit a high frequency co-inheritance, and are extremely useful in the identification of genetic mutations, such as, for example, mutations within the LIPIN3 gene, and the diagnosis of diseases and disorders related to mutations in the LIPIN3 gene.

Also, Caskey et al. (U.S. Pat. No. 5,364,759) describe a DNA profiling assay for detecting short tri and tetra nucleotide repeat sequences. The process includes extracting the DNA of interest, such as the LIPIN3 gene, amplifying the extracted DNA, and labeling the repeat sequences to form a genotypic map of the individual's DNA.

A LIPIN3 probe could additionally be used to directly identify RFLPs. Further, a LIPIN3 probe or primers derived from the LIPIN3 sequence could be used to isolate genomic clones such as YACs, BACs, PACs, cosmids, phage, or plasmids. The DNA contained in these clones can be screened for single-base polymorphisms or SSLPs using standard hybridization or sequencing procedures.

The level of LIPIN3 gene expression can also be assayed, as described above. For example, RNA from a cell type or tissue known, or suspected, to express the LIPIN3 gene, such as adipose tissue, testes, and normal placental tissue, may be isolated and tested utilizing hybridization or PCR techniques such as are described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the LIPIN3 gene. Such analyses may reveal both quantitative and qualitative aspects of the expression pattern of the LIPIN3 gene, including activation or inactivation of LIPIN3 gene expression.

Kits Comprising LIPIN3 Polynucleotides, Polypeptides and/or Antibodies

The present invention also encompasses kits containing LIPIN3 polynucleotide(s), polypeptide(s), and/or antibodies of this invention. The present invention also encompasses kits containing mutant LIPIN3 polynucleotide, and/or LIPIN3 polynucleotides suitable for the detection of mutant (altered) LIPIN3 polynucleotides. Diagnostic procedures using LIPIN3 polynucleotides, polypeptides and/or antibodies of this invention can be performed by diagnostic laboratories, experimental laboratories, practitioners, or private individuals. Kits embodied by this invention include those that allow someone to conduct an assay for the presence of LIPIN3 sequences, including mutant LIPIN3 sequences (e.g., mutant alleles), Lipin3 polypeptides and/or anti-Lipin3 antibodies, such as any of those disclosed herein, thus detecting an/or quantitating those activities. The kits encompassed by this invention may further provide instructions for any of the methods described herein, including detecting altered expression of a LIPIN3 polynucleotide and/or a Lipin3 polypeptide encoded by a LIPIN3 polynucleotide with altered expression. In another embodiment, the kits encompassed by this invention provide instructions for the detection of a mutant LIPIN3 polynucleotide or altered Lipin3 polypeptide, wherein the mutant polypeptide or polynucleotide is associated with obesity or an obesity related disease. The kits embodied by this invention also include kits that allow detection of LIPIN3 polynucleotides in, for example, ex vivo or in vivo transfected cells. These kits can be used for detection or quantitation of a polynucleotide that comprises a polynucleotide encoding a LIPIN3 or a portion thereof. Accordingly, the invention includes various kits.

In one embodiment, the invention provides a kit for detecting overexpression of a LIPIN3 polynucleotide comprising: a polynucleotide comprising: (i) a polynucleotide comprising the sequence shown in Table 1; (ii) a polynucleotide comprising the sequence shown in Table 7B; or (iii) a region of a polynucleotide comprising the sequence shown in Table 1 or Table 7B, wherein said region is at least about 15 polynucleotides in length (or more, e.g., at least about 18, 20, 25, 20, 50, 75, 100, or more); and instructions for using the region to assess LIPIN3 overexpression. In another embodiment, the invention provides a kit for detecting altered expression of a LIPIN3 polynucleotide comprising: a polynucleotide comprising (i) a polynucleotide having the sequence shown in Table 1; (ii) a polynucleotide having the sequence shown in Table 7B; or (iii) a region of a polynucleotide having the sequence shown in Table 1 or Table 7B, wherein said region is at least about 15 polynucleotides in length (or more, e.g., at least about 18, 20, 25, 20, 50, 75, 100, or more); and instructions for using the region to assess LIPIN3 expression.

The invention also provides kits for detecting mutant LIPIN3 polynucleotides and/or polypeptides. Such kits are useful for detecting a propensity for obesity or obesity related diseases, facilitating diagnosis of obesity and/or obesity diseases and the severity of obesity and/or obesity related diseases, and facilitating a determination of the prognosis of a subject. Detection can be based on detection of a polynucleotide that is differentially expressed in a cell from an individual with obesity or an obesity related disease and/or detection of a polypeptide encoded by a polynucleotide that is differentially expressed in cells from an individual with obesity. In one embodiment, the invention provides a kit for detecting a mutant LIPIN3 polynucleotide associated with obesity comprising a polynucleotide having the sequence shown in Table 8, or a region of a polynucleotide having the sequence shown in Table 8, wherein said region comprises nucleotide 1904.

The kits of this invention are in suitable packaging, and may optionally provide additional components that are useful in the procedure. These optional components include, but are not limited to, buffers, capture reagents, developing reagents, labels, reacting surfaces, means for detection, control samples, and interpretive information.

Methods using LIPIN3 Polynucleotides and Polypeptides: Screening Assays

The present invention also encompasses methods of identifying agents that may have anti-obesity activity based on their ability to reduce expression, especially overexpression of a LIPIN3 polynucleotide. These methods may be practiced in a variety of embodiments. We have observed that mouse LIPIN3 overexpression is associated with obesity in the BSB mouse, a model mouse with spontaneous, multifactorial obesity. While not being bound to any one theory, this observation suggests that a pathway(s) involving LIPIN3 polynucleotide overexpression may play a role in obesity and obesity-related diseases. Overexpression of the polypeptide encoded by overexpressed LIPIN3 polynucleotide may also play a role in obesity. We have also identified altered LIPIN3 polynucleotides associated with obesity in a population of obese women. Without being bound by theory, this observation suggests that mutations that alter Lipin3 activity and/or modulate LIPIN3 or Lipin3 expression may play a role in obesity and obesity related diseases.

The methods described herein are in vitro and cell-based screening assays. In the in vitro embodiments, an agent is tested for its ability to modulate expression of a LIPIN3 polynucleotide and/or a Lipin3 polypeptide. For the purposes of this invention, an agent may be identified based on any alteration of LIPIN3 expression, though characteristics associated with reduction of LIPIN3 overexpression may be preferable. Generally, overexpression is exemplified, but those descriptions and principles apply to detection or alteration of expression, if any. In the cell-based embodiments, living cells having LIPIN3 overexpression or expression of a LIPIN3 polynucleotide (including mutant LIPIN3 polynucleotides) are used for testing agents. For purposes of this invention, an agent may be identified on the basis of only partial loss of LIPIN3 overexpression, although total loss of LIPIN3 (over) expression may be preferable. An agent may also be identified on the basis of characteristics associated with reduction of LIPIN3 overexpression, including decreased expression of LIPIN3 polynucleotides, decreased expression of Lipin3 polypeptides, change in the number and/or size and/or accumulation of adipose cells.

In all of these methods, compromise of LIPIN3 overexpression may occur at any level that negatively affects LIPIN3 overexpression. An agent may compromise LIPIN3 overexpression by reducing or preventing transcription of LIPIN3. An example of such an agent is one that binds to the upstream controlling region, including a polynucleotide sequence or polypeptide. An agent may compromise LIPIN3 overexpression by reducing or preventing translation of LIPIN3 mRNA. An example of such an agent is one that binds to the mRNA, such as an anti-sense polynucleotide, or an agent which selectively degrades the mRNA. An agent may compromise Lipin3 overexpression by binding to Lipin3 or a Lipin3 polypeptide. An example of such an agent is a polypeptide or a chelator. An agent may compromise LIPIN3 overexpression by affecting the function of a protein that is in a Lipin3 pathway

In Vitro Screening Methods

In in vitro screening assays of this invention, an agent is screened in an in vitro system, which may be any of the following: (1) an assay that determines whether an agent is inhibiting expression, such as overexpression of LIPIN3; (2) an assay for an agent which interferes with expression, such as overexpression of LIPIN3 mRNA or a polynucleotide encoding Lipin3 or a Lipin3 polypeptide; (3) an assay for an agent that binds to Lipin3 or Lipin3 polypeptide. While the following discussion is focused on LIPIN3 polynucleotides and Lipin3 polypeptides, it is to be understood that this specificity is only for the purpose of simplicity and clarity. It is contemplated that the methods discussed below are applicable towards altered LIPIN3 polynucleotides and altered Lipin3 polypeptides.

For an assay that determines whether an agent inhibits transcription of LIPIN3, an in vitro transcription or transcription/translation system may be used. These systems are available commercially, and generally contain a coding sequence as a positive, preferably internal, control. A polynucleotide encoding Lipin3 (or a Lipin3 polypeptide), preferably containing LIPIN3 flanking sequences, is introduced and transcription is allowed to occur. Comparison of transcription products between an in vitro expression system that does not contain any agent (negative control) with an in vitro expression system that does contain agent indicates whether an agent is affecting LIPIN3 transcription. Comparison of transcription products between control and LIPIN3 indicates whether the agent, if acting on this level, is selectively affecting transcription of LIPIN3 (as opposed to affecting transcription in a general, non-selective or specific fashion).

For an assay that determines whether an agent inhibits translation of LIPIN3 mRNA or a polynucleotide encoding Lipin3 (or a Lipin3 polypeptide), an in vitro transcription/translation assay as described above may be used, except the translation products are compared. Comparison of translation products between an in vitro expression system that does not contain any agent (negative control) with an in vitro expression system that does contain agent indicates whether an agent is affecting LIPIN3 transcription. Comparison of translation products between control and LIPIN3 indicates whether the agent, if acting on this level, is selectively affecting translation of LIPIN3 (as opposed to affecting translation in a general, non-selective or specific fashion).

For an assay for an agent that binds to Lipin3 or Lipin3 polypeptide, LIPIN3 is first recombinantly expressed in a prokaryotic or eukaryotic expression system as a native or as a fusion protein in which the full length Lipin3 or regions of Lipin3 is conjugated with a well-characterized epitope or protein as described above under “Preparation of polypeptides of this invention”. Recombinant Lipin3 is then purified by, for instance, immunoprecipitation using anti-Lipin3 antibodies or anti-epitope antibodies or by binding to immobilized ligand of the conjugate. An affinity column made of Lipin3 or Lipin3 fusion protein is then used to screen a mixture of compounds which have been appropriately labeled. Suitable labels include, but are not limited to flurochromes, radioisotopes, enzymes and chemiluminescent compounds. The unbound and bound compounds can be separated by washes using various conditions (e.g. high salt, detergent) that are routinely employed by those skilled in the art. Non-specific binding to the affinity column can be minimized by pre-clearing the compound mixture using an affinity column containing merely the conjugate or the epitope. A similar method can be used for screening for agents that competes for binding to Lipin3 polypeptides. In addition to affinity chromatography, there are other techniques such as measuring the change of melting temperature or the fluorescence anisotropy of a protein which will change upon binding another molecule. For example, a BIAcore assay using a sensor chip (supplied by Biocore, Inc., Stitt et al. (1995) Cell 80: 661-670) that is covalently coupled to native Lipin3 or Lipin3-fusion proteins, may be performed to determine the Lipin3 binding activity of different agents.

In another embodiment, an in vitro screening assay detects agents that compete with another substance (most likely a polypeptide) that binds Lipin3 or a Lipin3 polypeptide. Competitive binding assays are known in the art and need not be described in detail herein. Briefly, such an assay entails measuring the amount of Lipin3 complex formed in the presence of increasing amounts of the putative competitor. For these assays, one of the reactants is labeled using, for example, ³²P.

It is also understood that the in vitro screening methods of this invention include structural, or rational, drug design, in which the amino acid sequence, three-dimensional atomic structure or other property (or properties) of Lipin3 (or Lipin3 polypeptide) provides a basis for designing an agent which is expected to bind to Lipin3 (or Lipin3 polypeptide). Generally, the design and/or choice of agents in this context is governed by several parameters, such as the perceived function of the Lipin3 (or Lipin3 polypeptide) target, its three-dimensional structure (if known or surmised), and other aspects of rational drug design. Techniques of combinatorial chemistry can also be used to generate numerous permutations of candidate agents. For purposes of this invention, an agent designed and/or obtained by rational drug designed may also be tested in the cell-based assays described below.

Cell-Based Screening Methods

In cell-based screening assays, a living cell containing a functioning LIPIN3 polynucleotide, a living cell containing a polynucleotide construct comprising a Lipin3 encoding sequence as described herein (including mutant LIPIN3 polynucleotide sequences), or genetically engineered cells in which LIPIN3 expression is increased or decreased or prevented, are exposed to an agent. In contrast (as described above), conventional drug screening assays have typically measured the effect of a test agent on an isolated component, such as an enzyme or other functional protein.

The cell-based screening assays described herein have several advantages over conventional drug screening assays: 1) if an agent must enter a cell to achieve a desired therapeutic effect, a cell-based assay can give an indication as to whether the agent can enter a cell; 2) a cell-based screening assay can identify agents that, in the state in which they are added to the assay system are ineffective to elicit at least one characteristic which is associated with compromise of LIPIN3 function, but that are modified by cellular components once inside a cell in such a way that they become effective agents; 3) most importantly, a cell-based assay system allows identification of agents affecting any component of a pathway that ultimately results in LIPIN3 expression, including overexpression. A cell based system also permits identification of agents affecting any component of a pathway that results in LIPIN3 overexpression in different genetic backgrounds, for example, in cells possessing mutations in other known obesity-implicated genes.

In one embodiment, an agent is identified by its ability to elicit modulation of LIPIN3 expression. In another embodiment, the modulation of LIPIN3 expression is reduction of LIPIN3 expression. In another embodiment, modulation of LIPIN3 expression includes reduction of LIPIN3 overexpression, a characteristic that we have associated with obesity in a mouse model for obesity. Thus, the invention provides a method of screening for agents that reduce the expression of a Lipin3 polynucleotide in a test cell sample comprising: contacting one or more cells possessing Lipin3 overexpression with an antiobesity drug candidate; monitoring expression of a LIPIN3 polynucleotide as described herein, and determining whether LIPIN3 expression has decreased.

In one embodiment, the invention provides methods for identifying an agent that may control obesity comprising the following steps: (a) contacting at least one agent to be tested with a suitable host cell that has LIPIN3 expression, preferably overexpression; and (b) analyzing LIPIN3 expression in said host cell, wherein an agent is identified by its ability to reduce LIPIN3 expression. For these methods, the host cell may be any cell in which LIPIN3 expression has been demonstrated. Examples of host cells include, but are not limited to, host cells as described herein. Host cells include cell lines as well as chronic cultures, in which host cells are explanted from an animal source, and cultured in vitro for a limited period of time, for example, adipose cells isolated from the BBS mouse and cultured in vitro. In another embodiment, the host cell is a genetically engineered cell in which LIPIN3 expression is increased, as described extensively above.

In one embodiment, a cell exhibiting LIPIN3 expression, preferably overexpression is contacted with at least one agent to be tested. The ability of this agent(s) to elicit at least one characteristic associated with compromise of LIPIN3 expression is then analyzed, and an agent is identified if at least one such characteristic is observed, e.g., increased accumulation of adipose cells, increased size of adipose cells, decreased accumulation of adipose cells, decreased size of adipose cells, increased or decreased maturity of adipose cells (which can be assessed by morphological examination known in the art, as well as study of cell-specific markers associated with stage of adipose development). In another embodiment, the host cell is a genetically engineered cell in which the native LIPIN3 gene has been replaced with an altered LIPIN3 gene, wherein the altered LIPIN3 gene correlates with obesity or an obesity related disease. In other embodiments, the genetically engineered cell further comprises an inducible promoter.

In another embodiment, the host cell is a host cell in which LIPIN3 expression is reduced or eliminated (which may be a genetically engineered host cell). Compounds that restore LIPIN3 expression (or characteristics associated with LIPIN3 expression) are candidates that mimic LIPIN3 activity or act in a LIPIN3 pathway. Use of an inducible promoter provides a means to determine whether the agent is acting via a LIPIN3 pathway. If an agent causes a characteristic indicative of loss of LIPIN3 function to appear in a cell in which the inducible promoter is activated, an observation that the agent fails to elicit the same result in a cell in which the inducible promoter is not activated indicates that the agent is affecting at least one step or aspect of LIPIN3 function. Conversely, if the characteristic indicating loss of LIPIN3 function is also observed in a cell in which the inducible promoter is not activated, then it can be assumed that the agent is not necessarily acting solely via the LIPIN3 functional pathway.

Cell-based screening assays of the present invention can be designed, e.g., by constructing cell lines in which the expression of a reporter protein, i.e., an easily assayable protein, such as β-galactosidase, chloramphenicol acetyltransferase (CAT), green fluorescent protein (GFP) or luciferase, is dependent on LIPIN3 function. For example, a gene under LIPIN3 control may have reporter sequences inserted within the coding region. The cell is exposed to a test agent, and, after a time sufficient to effect β-galactosidase or green fluorescent protein expression and sufficient to allow for depletion of previously expressed β-galactosidase or green fluorescent protein, the cells are assayed for the production of β-galactosidase or green fluorescent protein under standard assaying conditions. Expression of LIPIN3 polynucleotides and polypeptides may also be determined using the detection methods described herein.

Assay methods generally require comparison to a control sample to which no agent is added. Additionally, it may be desirable to use a cell partially or completely lacking LIPIN3 function as a control. For instance, if an agent were acting along a LIPIN3 pathway, one might expect to see the same phenotype as LIPIN3 cells treated with agents. If an agent were not acting along a LIPIN3 pathway, one may expect to see other characteristics that occur in the Lipin3 when treated with the agent.

The screening methods described above represent primary screens, designed to detect any agent that may reduce LIPIN3 expression or overexpression. The skilled artisan will recognize that secondary tests will likely be necessary in order to evaluate an agent further. A further screen is to perform an assay in an animal model, such as the BBS mouse model which develops a spontaneous multifactorial obesity. LIPIN3 overexpression is observed in BSB mice. Thus, one embodiment provides a method of administering a compound to BSB mice and monitoring the level of expression of a LIPIN3 polynucleotide. In addition, a cytotoxicity assay would be performed as a further corroboration that an agent which tested positive in a primary screen would be suitable for use in living organisms. Any assay for cytotoxicity would be suitable for this purpose, including, for example the MTT assay (Promega).

The following examples are provided to illustrate but not limit the present invention.

### EXAMPLES

### Example 1

### Identification of LIPIN3 Nucleotide Sequence

The coding sequence of human LIPIN3 was deduced using the reported mouse Lipin3 nucleotide sequence (GenBank Accession No. NM 022883) to search human sequence databases at NCBI/GenBank. The mouse Lipin3 amino acid sequence was also used to search GenBank Accession No. AL132654, revealing a partial, Lipin3 sequence. The mouse Lipin3 amino acid sequence was also used to search GenBank Accession No. NT 011382, which contains more that 2,400,000 base pairs of human chromosome 20. 609 amino acids of the predicted human Lipin3 amino acid sequence was identified, corresponding to 15 predicted exons in the genomic nucleotide sequence. These exons were used for allele discovery, as described in Example 5.

Subsequent database searches of the Celera human database revealed a predicted amino acid sequence of 846 amino acids for Lipin3, shown in Table 7A, and a predicted 2421 nt Lipin3 transcript, shown in Table 7B. The Lipin3 predicted transcript corresponds to 19 predicted exons, covering about the region from 38,239,865-38, 252, 935 bp on the Celera map of human chromosome 20 (as of Dec. 12, 2001). The Celera Otto computational predictions is missing the start codon.

A comparison of the mouse Lipin3 and the Celera human Lipin3 amino acid sequences revealed several gaps in the human Lipin3 sequence, totalling approximately 37 amino acids, suggesting that the predicted human Lipin3 amino acid sequence may be incomplete. The Celera Lipin3 amino acid sequence is 74% identical with the mouse Lipin3 amino acid sequence, and 47% and 43% percent identical with the human Lipin2 and Lipin1 amino acid sequences, respectively. Mouse Lpin3 has approximately 60% amino acid sequence homology to Lpin1, a gene involved in obesity, type 2 diabetes and atherosclerosis. See Peterfy et al., (2001) Nat Genet 27(1): 121-4. Two independent mutant alleles of Lpin1 are responsible for the spontaneous fatty liver dystrophy (fld) mutation. Fld mice have fatty livers, hypertriglyceridemia, insulin resistance, and diminished white and brown adipose depots. Lpin1 was found to be involved in adipocyte development and is localized in the nucleus.

We examined mouse and the Celera human lipin-3 amino acid sequences for predicted structural and amino acid motifs. Using Prosite (www.espasy.ch), we found multiple predicted PKC, CK2 and tyrosine phosphorylation sites, a cyclic AMP (cAMP) dependent phosphorylation site, multiple N-myristoylation sites and an amidation site. However, no N-terminal signal peptide sequence recognition, mitochondrial targeting sequence, endoplasmic reticulum retention motif, peroxisomal targeting signal, or RNA binding motif are predicted (http:://psort.nibb.acjp/cgi-bin). We also identified sequences for nuclear localization (NLS) that are well conserved within the Lipin gene family in both human and mouse. Sequences surrounding the NLS appear to be distinctive for each lipin subtype that is conserved between human and mouse suggesting functional relevance. Differences between the B6 and Spretus lipin 3 sequence are found near the NLS. Results from k-NN Prediction showed human lipin 3 likelihood to be 82.6% nuclear, 13.0% cytoskeletal or 4.3% vesicles of secretory system (as determined using PSORTII). Using the GOR4 secondary structure prediction method version IV (see Gamier et al., Methods in Enzymology (1996) R. F. Doolittle Ed., vol 266, 540-553) Lipin is predicted to be mostly random coil (59.06%) with some alpha helix (23.70%) and extended strand regions (17.25%).

### Example 2

### Expression Analysis of Human LIPIN3 Nucleotide Sequence

Expression analysis was performed by electronic searching of sequence databases obtained by sequence analysis of cDNAs. In overview, databases containing nucleic acid sequence derived from cDNA libraries were searched with the Celera Lipin3 amino acid sequence. Matching sequences were identified, and the source cDNA of the matching sequences was determined.

The Lipin3 amino acid sequence was used to search the human EST databases (http://www.ncbi.nlm.nih.gov/) and the following EST clones with identical sequences were identified from UniGene cluster Hs.302510:

| Number | name | Source of cDNA |
|---|---|---|
| AI208765 | cDNA clone IMAGE: | testis 3′ read 0.6 kb |
| | 1837448 | |
| BG009114 | cDNA clone (no-name) | placenta_normal |
| AL449688 | cDNA clone (no-name) | liver |
| AL449687 | cDNA clone (no-name) | liver |
| BE837827 | cDNA clone (no-name) | prostate_normal |
| B1768204 | cDNA clone IMAGE5205806 | pooled lung and 5′ read spleen |
| BE59518 | cDNA clone (no-name) | head_neck |

A BlastN search also identified addition EST sequences not found in the UniGene cluster, with the following GenBank Accession Nos.: Genbank BF759638, BF872665, and BG009242.

This data indicates that human LIPIN3 is expressed at least in placenta, prostrate, liver, and testes tissue.

Expression analysis was also performed using hybridization analysis. GenBank Accession number A1208765, which contains partial human LIPIN3 cDNA sequences, was used to probe a human-tissue mRNA sample array (Clontech #7776-1), as directed by the manufacturers. Detectable expression was observed in a variety of tissues, including heart, thyroid, adrenals, liver and pancreas (data not shown).

### Example 3

### Determining the Map Position of the Human and mouse LIPIN3 Genes

The human LIPIN3 gene was mapped to a chromosomal location at 46.1 MB on human chromosome 20, approximately 600,000 base pairs distal to D20S108. Lipin3 is found at bases 38,239,865-38, 252, 935 on the Celera human map (as of Dec. 12, 2001). By contrast, D20S108 is placed at 39,100,000 on the Celera Human map, or about 900,000 bp distal to Lipin3.

Human LIPIN3 maps directly under the peak for linkage of a quantitative trait locus (“QTL”) for body mass index (“BMI”) in a population of 1,711 obese individuals in 103 extended pedigrees. See Hunt et al. (2001), Hum. Genet. 109(3): 279-85. These data indicate that human LIPIN3 gene maps to a region that is statistically associated with increased body mass index in a population of obese women.

The mouse LIPIN3 gene was mapped to a 90.4 cM region on mouse chromosome 2, between Nnat (88 centimorgans on the JAX map) and D2Mit263 (92 centimorgans on the JAX map). This map positions corresponds to a QTL controlling body fat and temperature in the BSB mouse, a model mouse that develops spontaneous multifactorial obesity. See generally Fisler et al., Obes. Res. 1:271-280 (1997); Fisler & Warden, J. Nutrition 127:1909s-1916S (1997). We have also identified a mouse obesity gene in this region in studies of congenic mouse strains (data not shown). Mouse chromosome 2 is syntenic, or homologous, to human chromosome 20. Thus, the observed linkage of obesity to these chromosomes may implicate the same gene in causing obesity in both species.

### Example 4

### Expression Analysis of Mouse LIPIN3 Gene, Expression of LIPIN3 in Obese BSB Mice, and Analysis of Differentially Expressed Genes in Obese Verses Lean BSB Mice

A mouse IMAGE clone (4023808) encoding a mouse lipin-3 cDNA was obtained. Following sequencing to confirm the identity as a lipin-3 cDNA, the clone was then used as a probe on a Northern blot of RNAs from normal mouse tissues. 20 μg of total RNA from the following tissues of male and female C57BL/6J mice was used to produce the Northern blot: gastrocnemius muscle (lane 1); Brain (lane 2); Spleen (lane 3); Duodenum (lane 4); Kidney (lane 5); Lung (lane 6); Heart (lane 7); Liver (lane 8); Gonadal white adipose tissue (lane 9); and femoral white adipose tissue (lane 10). Hybridization to an approximately 4.7 kb band was observed in brain, skeletal muscle, heart, and adipose tissue.

These results demonstrate that the mouse Lipin-3 gene has a full length mRNA of approximately 4.7 kb. They also show that the mRNA is expressed in brain, skeletal muscle, heart and adipose tissue.

We also analyzed expression of LIPIN3 in an animal model for obesity, BSB mice that develop a spontaneous multifactorial obesity. Briefly, BSB mice are produced by a backcross of the lean inbred C57BL/6J (B6) strain with lean inbred Mus spretus (SPRET/Ei). The lean F1s are then backcrossed to B6 to produce the BSB mice. Every BSB mouse is unique—total body fat varies from 1 up to 50%. We compared the level of LIPIN3 RNA expression in lean and obese BSB mice. mRNA was isolated from the gonadal adipose tissue from seven pairs of lean and obese BSB mice. Each mRNA isolate was hybridized to a glass slide microarray (obtained from Lawrence Berkeley Labs) that contained pcr products from more than 10,000 mouse Unigenes, including an EST clone encoding a portion of the mouse Lipin3 cDNA sequence Hybridization patterns (including relative intensity of hybridization), were compared between lean BSB mice and obese BSB mice.

Mouse adipose RNA hybridized to an EST corresponding the mouse Lipin3 gene, indicating that mouse LIPIN3 is expressed in adipose tissue. Increased hybridization was observed with RNA isolated from obese BSB mice relative to the hybridization of LIPIN3 isolated from lean BSB mice, indicating that expression of LIPIN3 RNA is approximately two-fold increased in obese BSB mice relative to lean BSB mice. Quantititive PCR experiments were carried out as follows: RNA was prepared from gonadal adipose tissues of the 10 leanest and the 10 most obese male BSB mice from a sample of over 600 mice using TRIzol reagent (Gibco/BRL, Grand Island, N.Y.) and quantified. RNA quality and integrity was confirmed by 1% denaturing gel electrophoresis. Complementary DNA was generated from 1 μg of total RNA as instructed by the manufacturer (Applied Biosystems, Foster City, Calif.). Amplification primer pairs were specifically designed for the genes of interest (Primer Express software Applied Biosystems, Foster City, Calif.). Quantitative real-time PCR reactions were carried out with 30 ng of cDNA using SYBR Green I dye detection of the ABI PRISM 7900HT Sequence Detector System (Applied Biosystems, Foster City, Calif.). The reagents and amplification conditions were used as suggested by the manufacturer. Fold changes in expression levels were analyzed by analysis of variance (ANOVA) (StatView 5.0, Abacus Concepts Inc., Berkeley, Calif.). These quantitative PCR experiments revealed that obese BSB mice have approximately 4.4-fold higher expression of LIPIN3 than do non-obese BSB mice. Fold changes are statistically significant (P<0.05).

The mouse LIPIN3 sequence was used to search the Unigene databases containing mouse cDNA sequences. See http://www.ncbi.nlm.nih.gov/UniGene. Unigene Mm.61792 Mus musculus was identified as the mouse Unigene corresponding to mouse LIPIN3.

We also identified other genes that are differentially expressed in BSB adipose tissue of obese verses lean BSB mice. Briefly, mRNA was isolated from the gonadal adipose tissue from seven pairs of lean and obese BSB mice. Each RNA isolate was hybridized to a glass slide microarray obtained from Lawrence Berkeley Labs. Each slide contained pcr products from more than 10,000 mouse Unigenes, including an EST clone encoding a portion of the mouse Lipin3 cDNA sequence hybridization patterns (including relative intensity of hybridization), were compared between lean BSB mice and obese BSB mice as described above. We observed 65 genes were over expressed at least two-fold in obese verses lean BSB mice, and 14 genes were under expressed at least two fold in lean verses obese BSB mice. Table 11 shows a selection of these genes. The chromosomal location of all the differentially expressed genes from the BSB array were mapped using the whole mouse genome sequence from Celera, and differentially expressed genes were correlated with genes described by other investigators as differentially expressed in adipose from lean and obese mice. See, eg, Soukaset al. (2000) Genes Dev 14(8): 963-80; Nadler et al. (2000) Proc Natl Acad Sci USA 97(21): 11371-6.

TABLE 11
Differentially expressed genes in BSB gonadal adipose tissue
that map to BSB obesity QTLs (SEQ ID NO:14).

| | | Location | | Over- |
|---|---|---|---|---|
| Description | Accession No | Chr. | cM | expressed in |
| Leptin | AA510287 | 6 | 10.5 | obese * |
| Transcription | W98752 | 6 | 11 | obese |
| elongation factor A | | | | |
| (SII), I | | | | |
| Lipopolysaccharide | W09930 | 2 | 83 | obese * |
| binding protein (LBP) | | | | |
| Lipin-3 | | 2 | 90 | obese |
| Molybdopterin | W99918 | 2 | 97 | obese |
| synthase sulfurylase | | | | |
| (Mocs3) | | | | |
| Kinesin family | AA209952 | 2 | 86 | lean |
| member 3b (kif3B) | | | | |

* Also reported by Soukas et al. (2000) Genes Dev 14(8): 963-80; Nadler et al. (2000) Proc Natl Acad Sci USA 97(21): 11371-6.

Gene positions were obtained using both sequence and text based searches. Chromosomal locations reported by Celera were obtained via translated BLAST sequence queries, using a maximal E-value threshold of 10⁻⁴. In all cases for the obesity candidates listed in Table 11, E-values were below 10⁻³⁰, with a minimal sequence identity of 75% direct base pair lineup. Locations reported in centimorgans were obtained from the Jackson Laboratory bioinformatics database, which permits searches based on gene name or description. See http:///www.jax.com. The EST accession number with which a gene is associated may also span the regions of multiple expressed sequence tags. Hence, the same gene may be represented by different sequence accession numbers. We attempted to encompass the entire range of possible representative EST's for a particular gene by comparing for the presence of different EST's within a single UNIGENE. In cases where two previously distinct EST's were found to be grouped within the same UNIGENE, it was concluded that the genes affiliated with those EST's may also share homology, or in some cases, the same identity. When possible, genes called by the Celera Discovery System database were accepted as verifiable and conclusive. Accession numbers from differentially expressed genes provided the means to obtain the FASTA format nucleotide sequence from the National Center for Biotechnology Information (NCBI). The FASTA sequence was then entered into a nucleotide translated BLAST (tBLASTN) via Celera, and the resultant gene calls were screened for identity, with a maximal E-value threshold of 10⁻⁴. In all cases for the obesity candidates listed above, E-values were below 10⁻³⁰, with a minimal sequence identity of 75% direct base pair lineup. Names for genes were taken from Celera's identification for matching gene identities, and further verified for authenticity by querying the sequence through PSI-BLAST. Although PSI-BLAST matches denote homology, we accepted matches with E-values below 10⁻³⁰ that corroborated our findings from Celera.

### Example 5

### Identification of Human LIPIN3 Polymorphisms in a Population of Obese Women

This Example describes the identification of polymorphisms in human LIPIN3 coding region, and the statistical association of one polymorphism with obesity-related phenotypes in a population of obese women.

71 genomic DNA samples were obtained from N. Butte at the Children's Children's Nutrition Research Center (CNRC), Houston, Tex. This study was approved by the Baylor Affiliates Review Board for Human Subject Research, and informed written consent was obtained from each woman. Briefly, a study population of 71 unrelated women (Butte study No. 180) was collected in order to study the effects of pregnancy on energy expenditure. This study was designed to determine the effect of age, body composition, and fitness on energy requirements of healthy women with varying body mass index (hereinafter, “BMI”). Low BMI was defined as <18.5, normal BMI as >18.5 but <25, and high BMI as >25. Enrollment criteria included nonsmoking, ages 18-39 y, parity not greater than 4, physically active (i.e., 20-30 minutes of moderate exercise at least three times per week), and no chronic medications or alcohol or drug abuse. The women were normotensive, glucose tolerant, nonanemic and euthyroidic. Measurements of anthropometry, body composition, basal metabolic rates, total energy expenditure, fitness and strength were performed at the Children's Nutrition Research Center (CNRC), Houston, Tex. The women range in age from 21 to 39 years (mean=31) and have a mean BMI of 22. For a description of methods used to access body composition (e.g., fat mass, lean mass) and energy expenditure, see, e.g., Hopkinson et al. (1997) Am J Clin Nutr 65(2): 432-8 and Butte et al. (1999) Am J Clin Nutr 69(2): 299-307.

In overview, we identified 5 polymorphisms in the human lipin 3 gene by amplifying 15 exons. We then performed an association study by genotyping one common polymorphism in the study population described above.

Oligonucleotide primer pairs corresponding to each LIPIN3 exon were designed. The sequence of the primers is shown in Table 12 (see also FIG. 1).

TABLE 12

| | | |
|---|---|---|
| Primer name | Primer sequence | SEQ ID NO: |

| | | |
|---|---|---|
| Lipinex1f: | ttctttgccacgaccagc | (SEQ ID NO:15) |
| Lipinex1r: | cagccagcctgagcactc | (SEQ ID NO:16) |
| Lipinex2f: | agggagagatgggtagggag | (SEQ ID NO:17) |
| Lipinex2r: | gaagaggcacaggaaaggg | (SEQ ID NO:18) |
| Lipinex3f: | tgggtaggcaggctcaag | (SEQ ID NO:19) |
| Lipinex3r: | ggcctggggttgatacag | (SEQ ID NO:20) |
| Lipinex4f: | cagaaggttctgctgggc | (SEQ ID NO:21) |
| Lipinex4r: | gatccagaacccaggcac | (SEQ ID NO:22) |
| Lipinex5f: | cttgtcattgttggcccc | (SEQ ID NO:23) |
| Lipinex5r: | tcacagtcttccctgccc | (SEQ ID NO:24) |
| Lipinex6f: | cagcctcacccctcactc | (SEQ ID NO:25) |
| Lipinex6r: | attgactctccggctgcc | (SEQ ID NO:26) |
| Lipinex7f: | ccctcttcacaaccctgc | (SEQ ID NO:27) |
| Lipinex7r: | tctgctcacccttcacttcc | (SEQ ID NO:28) |
| Lipinex8f: | catggctttcatctgccc | (SEQ ID NO:29) |
| Lipinex8r: | cctcgactgtgaccaccc | (SEQ ID NO:30) |
| Lipinex9f: | cagcctcctcttggcatc | (SEQ ID NO:31) |
| Lipinex9r: | tccccaacctccacacag | (SEQ ID NO:32) |
| Lipinex10f: | caatcacgacacaccccc | (SEQ ID NO:33) |
| Lipinex10r: | acaacccctctccctccc | (SEQ ID NO:34) |
| Lipinex11f: | tctgtgctgggtgtggtg | (SEQ ID NO:35) |
| Lipinex11r: | tcctgtcctgtcactccctc | (SEQ ID NO:36) |
| Lipinex12f: | accctgtcccccactctc | (SEQ ID NO:37) |
| Lipinex12r: | ctgaccccaccctccttg | (SEQ ID NO:38) |
| Lipinex13f: | aggagggtggggtcagac | (SEQ ID NO:39) |
| Lipinex13r: | ctcagcagagtggagggg | (SEQ ID NO:40) |
| Lipinex14f: | gtgtcccctccactctgc | (SEQ ID NO:41) |
| Lipinex14r: | ctcgaccacttcaccaagc | (SEQ ID NO:42) |
| Lipinex15f: | actgtttggtggcagctc | (SEQ ID NO:42) |
| Lipinex15r: | gcctcctccttccccttc | (SEQ ID NO:44) |

15 exons were amplified from the genomic DNA of the study patients. Candidate polymorphisms in human Lipin-3 were identified by using temperature-modulated heteroduplex analysis (TMHA), essentially as described in Hecker et al. (2000) Cell Stress Chaperones 5(5): 415-24. Amplicons amenable to mutation detection by denaturing HPLC were identified based on the predicted melting profile of the target sequence generated using WAVEMAKER software (Transgenomics, Inc.). TMHA was performed on PCR products of individual amplicons on the WAVE Nucleic Acid Fragment Analysis System (Transgenomics Inc.) as described in Hecker et al., above.

Candidate polymorphisms were confirmed by direct sequencing of the PCR products. Table 13 describes five single nucleotide polymorphisms (hereinafter, “SNPs”) that were identified, including the approximate frequency of the SNP, the location of the SNP (intron or exon), the change in the polynucleotide sequence (base pair), the change in the amino acid. The polynucleotide and amino acid numbers on the table correspond to the Celera polynucleotide and amino acid sequences shown in Tables 7A and 7B. SNPs labeled as “common” are SNPs observed in greater than 10% of the samples. For example, the Gln allele of the polymorphism His 634Gln was found in 71% of samples and the His allele was found in 29% of samples. “Rare” SNPs are SNPs observed in less than 10% of the samples. For example, the polymorphisms in sample 12 and 72 were found in 1/91 samples (but in different samples).

TABLE 13
SNPs identified by direct sequencing of PCR products
amplified from genomic DNA of study patients.

| Sample # | Frequency | SNP location | bp Change | aa Change |
|---|---|---|---|---|
| 12 | Rare | Intron | | |
| 72 | Rare | Intron | | |
| 33 | N/A* | Intron | | |
| 12 | N/A | Intron | | |
| 33 | Common | Exon | A 1904 C | Gln634His |

*The frequency in these samples may be common, but WAVE analysis was not definitive and these polymorphisms were not characterized further.

Table 14 shows the size of the SNP-containing PCR product, the location of the SNP when the DNA sequence of the PCR product was determined, and the primer set used for the amplification.

TABLE 14

| | | location of SNP | | |
|---|---|---|---|---|
| | | (polymorphic base) | | |
| | | (numbers refer to position | | |
| | | within amplified pcr product) | | |
| Sample # | PCR length | Forward | Reverse | Primer set |
| 12 | 355 | 190 (A/G) | 143, (T/C) | 2 |
| 72 | 355 | 329, (T/C) | 4, (A/G) | 2 |
| 33 | 205 | 181, (C/T) | −1, (G/A) | 10 |
| 12 | 250 | 31, (T/C) | 191, (G/A) | 11 |
| 33 | 250 | 177, (C/A) | 46, (G/T) | 11 |

Association Analysis of the Polymorphism from Sample 33 (Interchangeably Termed SNP11-33, or His634Gln)

Further analysis of the His634Gln polymorphism was conducted by performing an association study in the population of women described above. The polymorphism represents an A1904C substitution at position 1904 (in the Celera nucleotide sequence, see Table 7B). Individuals homozygous for the “C” allele showed significantly elevated body weight (P=0.05), waist circumference ({=0.04), fat mass (P=0.004), % fat (P=0.01), triglyceride (P=0.04) and leptin levels (P=0.02) than heterozygous individuals, and significantly lower energy expenditure (MAX VO2) (P=0.04) than heterozygous individuals. This data strongly supports the conclusion that human LIPIN3 is the human obesity gene.

### Example 6

### Linkage Studies Identify Candidate Obesity Genes on Mouse Chromosome 2

Linkage analysis was performed between percent body fat and markers on chromosome 2 (73.9-107 cM) in the BSBHLKO cross described in Example 4. There was strong linkage across the entire region that includes several candidate genes, including lipopolysaccharide binding protein (LBP), molybdopterin synthase sulfurylase (Mocs3) and Lpin3. As noted above, these genes were overexpressed in adipose tissue of obese relative to lean BSB mice.

Table 15 shows the linkage map for chromosomal 2 locus linked to % body fat in BSB mice. The locations of some candidate genes are indicated. 745 BSB mice of both genders and all HL genotypes were genotyped as BB or SB, using polymorphic markers or coat color (agouti) on Chr 2. D2Mit106: n=733, BB=16.1±0.5, SB=13.3±0.4, F-Test=17.3. D2Mit109: n=694, BB=16.2±0.5, SB=12.9±0.4, F-Test=24.8. Coat color: n=745, BB=16.1±0.5, SB=13.0±0.4, F-Test=23.7. D2Mit229: n=691, BB=15.8±0.5, SB=13.0±0.4, F-Test=18.3. D2Mit74: n=507, BB=15.8±0.6, SB=13.5±0.5, F-Test=9.1.

TABLE 15
Linkage map for chromosomal 2 locus linked to % body fat in
BSB mice.

| cM | Chr. 2 | name |
|---|---|---|
| 73.9 | | attractin |
| 75.6 | | D2Mit106*** |
| 81.7 | | D2Mit109*** |
| 83 | | LBP |
| 86 | | Kif3b |
| 89 | | agouti*** |
| 90.4 | | Lpin3 |
| 96 | | Mocs3 |
| 99 | | D2Mit229*** |
| 100 | | Mc3r |
| 107 | | D2Mit74** |

Abbreviations: LBP (lipopolysaccharide binding protein), Kif3b (kinesin family member 3b), Lpin3 (lipin3), Mocs3 (molybdopterin synthase sulfurylase), Mc3r (melanocortin 3 receptor).
Data are shown as means ± SE. ***P ≦ 0.0001, **P = 0.003.

### Example 7

### Identification of Polymorphisms in B6 and Spretus Mice

Genomic DNA was isolated from kidney (QIAamp Blood kit and QIAamp Tissue kit, QIAGEN Inc., Chatsworth, Calif.). Ninety-six BSB mice were genotyped with the Pyrosequencing system (Pyrosequencing AB, Westborough, Mass.) for the lipn3 gene, using two of the identified SNP polymorphisms (G1287A and C1489A) between the B6 and spretus strains. Briefly, 25 ul of a 200 bp amplicon including the SNP was immobilized on streptavidin beads (Dynal A/S, Norway) and denatured with NaOH. Sequencing primer was annealed and the samples were processed in an automated pyrosequencing instrument.

Simple sequence length polymorphic (SSLP) markers, polymorphic between B6 and spretus strains, were used to genotype the mouse chromosome 2 region (Research Genetics, Huntsville, Ala.). Fifty ng of genomic DNA was amplified in a 10 μl reaction using AdvanTaq PlusDNA polymerase (Clontech, cat.# 8431-1, Palo Alto, Calif.). Samples were amplified on a “Robocycler” thermocycler (Stratagene, La Jolla, Calif.) under the following conditions: one cycle at 94° C. for 1 min, followed by thirty cycles at 94° C. for 30 s, 52° C. for 30 s, and 68° C. for 30 s, followed by one cycle at 72° C. for 5 min. PCR products were resolved by electrophoresis in a 2% agarose gel (GibcoBRL, Life Technologies Inc., Gaithersburg, Md.) and visualized by ethidium bromide staining. The coding region of Lpin3 for B6 (2547 bp) and spretus (2544 bp) were sequenced to at least four-fold each. B6 and spretus alleles of Lpin3 were genotyped on a Pyrosequencer using the SNPs, G1287A and C1489A. Two Lpin3 SNPs were chosen to type the BSB mice to ensure consistency. Each SNP chosen was unique to either flanking base, thus, increasing reliability of chromosomal mapping and haplotyping results.

Twenty-eight nucleotide polymorphisms and five non-conserved amino acid changes were identified as shown in Table 16.

TABLE 16
Lpin3 polymorphisms identified for B6 and spretus. Base and
amino acid position are given in terms of B6 sequence

| Polymorphism B to S | Amino acid change |
|---|---|
| GGG deletion | Deletion of Glu139 |
| C 551 A | Ala184Glu |
| C 902 A | Tyr301Lys |
| C 1262 T | Pro421Leu |
| C 1489 A | Leu497Met |

Interestingly, the GGG deletion (corresponding to a Glu deletion) lies within 5-10 amino acids of the prediced nuclear localization signal of the Lipin3 protein.

Although the foregoing invention has been described in some detail by way of illustration and example for purposes of clarity of understanding, it will be apparent to those skilled I the art that certain changes and modifications can be practiced. Therefore, the description and examples should not be construed as limiting the scope of the invention, which is delineated by the appended claims.

We claim:

1. An isolated polynucleotide comprising (a) the sequence shown in Table 8, or its complement; (b) a polynucleotide that selectively hybridizes to the sequence shown in Table 8 relative to a known polynucleotide; (c) a region of at least about 15 contiguous nucleotides, said region comprising nucleotide 1904 shown in Table 8; or (d) a polynucleotide that selectively hybridizes to the sequence shown in Table 8 relative to a known polynucleotide.

2. The polynucleotide of claim 1, wherein the region is at least about 20 contiguous nucleotides.

3. A polynucleotide according to claim 1, said polynucleotide further comprising a detectable label.

4. A polynucleotide according to claim 1, wherein said polynucleotide is immobilized on a surface.

5. A polynucleotide according to claim 1, wherein said polynucleotide is single stranded.

6. A polynucleotide according to claim 1, wherein said polynucleotide is selected from the group consisting of DNA and RNA.

7. A polynucleotide according to claim 1, wherein said polynucleotide is prepared in part by chemical synthesis.

8. A vector comprising the polynucleotide according to claim 1.

9. An expression vector comprising the polynucleotide according to claim 1.

10. A host cell comprising the polynucleotide according to claim 1.

11. An isolated polypeptide comprising: a polypeptide comprising (i) a polypeptide having the sequence shown in Table 10; or (ii) a region consisting at least about 5 contiguous amino acids, wherein said region includes amino acid 634 of Table 10.

12. The polypeptide according to claim 11, wherein the polypeptide includes an epitope.

13. The polypeptide according to claim 11, wherein the polypeptide is immobilized on a solid support.

14. A method for detecting differential expression of a LIPIN3 polynucleotide in a test sample comprising: detecting a level of expression of (i) a polynucleotide comprising the sequence shown in Table 1; (ii) a polynucleotide comprising the sequence shown in Table 7B; or (iii) a region of a polynucleotide having the sequence shown in Table 1 or Table 7B, wherein said region is at least about 10 nucleotides in length.

15. A method for detecting obesity or obesity-related disorders associated with differential expression of a LIPIN3 polynucleotide comprising:
(a) detecting a level of expression of (i) a polynucleotide comprising the sequence shown in Table 1; (ii) a polynucleotide comprising the sequence shown in Table 7B; or (iii) a region of a polynucleotide having the sequence shown in Table 1 or Table 7B, wherein said region is at least about 10 nucleotides in length.

16. The method of claim 14 or 15, wherein the differential expression is elevated expression.

17. The method of claim 14 or 15, wherein the detecting is measuring the level of an RNA transcript.

18. The method of claim 14 or 15, wherein the detecting is by a method including PCR, TMA, bDNA, NAT, or Nasbau.

19. The method of claim 14 or 15, wherein the polynucleotide is immobilized on a surface.

20. A method of detecting a polynucleotide polymorphism associated with obesity, comprising: detecting a polynucleotide according to claim 1.

21. A method of diagnosing obesity or an obesity-related disorder comprising detecting a polynucleotide according to claim 1.

22. A method for detecting differential expression of a LIPIN3 polypeptide in a test sample comprising: detecting a level of expression of (i) a polypeptide comprising the sequence shown in Table 9; (ii) a polypeptide comprising the sequence shown in Table 7A; or (iii) a region of the polypeptide shown in Table 9 or Table 7A, wherein the region is at least 10 contiguous amino acids in length.

23. The method of claim 22, wherein the differential expression is overexpression.

24. The method of claim 22, wherein the detecting is by a method including western blotting, mass spectroscopy, ELISA, immunohistochemistry, and capillary electrophoresis.

25. A method of screening for agents that reduce the expression of a LIPIN3 polynucleotide in a test cell sample comprising:
contacting one or more cells possessing LIPIN3 overexpression with an antiobesity drug candidate;
measuring expression of: (i) a polynucleotide comprising the sequence shown in Table 1; (ii) a polynucleotide comprising the sequence shown in Table 7B; or (iii) a region of a polynucleotide having the sequence shown in Table 1 or Table 7B, wherein said region is at least about 15 nucleotides in length;
and determining whether expression of the polynucleotide has decreased.

26. The method of claim 25, wherein an amount of the polynucleotide is measured by detecting mRNA in said sample.

27. The method of claim 26, wherein expression is measured by hybridizing said mRNA to a probe that specifically hybridizes to the polynucleotide.

28. The method of claim 27, wherein said hybridizing is according to a method selected from the group consisting of a Northern blot, a Southern blot, an array hybridization, an affinity chromatography, and an in situ hybridization.

29. The method of claim 27, wherein said probe is a member of a plurality of probes that forms an array of probes.

30. The method of claim 26, wherein the level of expression is measured using a nucleic acid amplification reaction.

31. The method of claim 25, wherein the cell is cultured ex vivo.

32. The method of claim 25, wherein said cell is an adipocyte.

33. The method of claim 25, wherein the test agent is selected from the group consisting of antibody; protein; nucleic acid; and small organic molecule.

34. A method of screening for agents that reduce the expression of a Lipin3 polypeptide in a test cell sample comprising:
contacting one or more cells possessing Lipin3 overexpression with an antiobesity drug candidate;
measuring expression of: (i) a polypeptide comprising the sequence shown in Table 9; (ii) a polypeptide comprising the sequence shown in Table 7A; or (iii) a region of the polypeptide shown in Table 9 or Table 7A, wherein the region is at least 10 contiguous amino acids in length;
and determining whether Lipin3 expression has decreased.

35. The method of claim 34, wherein said measuringing is via a method selected from the group consisting of capillary electrophoresis, a Western blot, mass spectroscopy, ELISA, immunochromatography, and immunohistochemistry.

36. The method according to claim 34, wherein said cell is cultured ex vivo.

37. The method according to claim 34, wherein said cell is an adipocyte.

38. The method according to claim 34, wherein the test agent is selected from the group consisting of antibody; protein; nucleic acid; and small organic molecule.
